(19) United States
(12) Patent Application Publication
(10) Pub. No.: US 2010/0196398 A1
(43) Pub. Date: Aug. 5, 2010

(54) TARGETED BINDING AGENTS DIRECTED TO CD105 AND USES THEREOF

(75) Inventors: GADI GAZIT-BORNSTEIN, WALTHAM, MA (US); NAOMI LAING, WALTHAM, MA (US); SIMON THOMAS BARRY, CHESHIRE (GB); JOHN BABCOOK, British Columbia (CA); QING ZHOU, FREMONT, CA (US)

Correspondence Address: MEDIMMUNE, LLC;Patrick Scott Alban, ONE MEDIMMUNE WAY, GAITHERSBURG, MD, 20878, US

(73) Assignee: MedImmune LLC, Gaithersburg, MD (US)

(21) Appl. No.: 12/562,533

(22) Filed: Sep. 18, 2009

Related U.S. Application Data
(60) Provisional application No. 61/098,685, filed on Sep. 19, 2008.

(51) Int. Cl. A61K 39/395; C07K 16/18; C07H 21/00; A61P 35/00

(52) U.S. Cl. 424/172.1; 530/387.1; 530/388.15; 536/23.53

(57) ABSTRACT

The invention relates to targeted binding agents against CD105 and uses of such agents. More specifically, the invention relates to fully human monoclonal antibodies directed to CD105. The described targeted binding agents are useful in the treatment of diseases associated with the activity and/or overproduction of CD105 and as diagnostics.

### CROSS REFERENCE TO RELATED APPLICATION

This application claims priority to and under the benefit of U.S. Provisional Patent Application No. 61/098,685 filed on Sep. 19, 2008, the entire disclosure of which is incorporated by reference herein.

### FIELD OF THE INVENTION

The invention relates to targeted binding agents against CD105 and uses of such agents. More specifically, the invention relates to fully human monoclonal antibodies directed to CD105. The described targeted binding agents are useful in the treatment of diseases associated with the activity and/or overproduction of CD105 and as diagnostics.

### BACKGROUND OF THE INVENTION

CD105, otherwise referred to as endoglin, is a transmembrane glycoprotein expressed on activated vascular endothelial cells (Letamendia A, Lastres P, Botella L M, et al. J Biol Chem 1998; 273:33011-9). CD105 has also been reported to be highly expressed on tumor vasculature, and weakly on a limited number of other cell types, including macrophages, fibroblasts, and syncytiotrophoblasts (Fonsatti E et al., Oncogene 2003: 22:6557-6563).

CD105 is composed of two disulfide-linked subunits of 95 kDa each, forming a 180-kDa homodimeric protein (Barbara N P, Wrana J L, Letarte M. J Biol Chem 1999; 274:584-94). The CD105 gene is 40 kb in length and located on human chromosome 9q34 (Fonsatti E, Sigalotti L, Arslan P, Altomonte M, Maio M. Curr Cancer Drug Targets 2003; 3:427-32; Rius C, Smith J D, Almendro N, et al. Blood 1998; 92:4677-90). The mRNA transcript is 3.4 kb in length and consists of 14 exons. Exons 1 to 12 encode the extracellular domain, while the transmembrane domain is encoded by exon 13, and the cytoplasmic domain by exon 14. Two different isoforms of CD105 have been identified, designated long (L-CD105/endoglin) and short (S-CD105/endoglin). The aforementioned isoforms differ in amino acid composition within the cytoplasmic tails. More specifically, the L isoform is predominant, and contains 47 residues in the cytoplasmic domain, whereas the S isoform contains only 14 amino acids (Gougos A, Letarte M. J Biol Chem 1990; 265: 8361-8364; Lastres P et al. Biochem J, 1990; 301:765-768).

CD105 is a co-receptor for the transforming growth factor-β (TGF-β) receptor; it forms heterodimers with the signaling type I and type II receptors of TGF-β and can modulate responses to TGF-β (Yamashita H et al., J Biol Chem, 1994; 269:1995-2001; Guerrero-Esteo M et al., J Biol Chem 2002; 277:29197-29209). TGF-β is a cytokine that is part of a larger superfamily of proteins that include activins and bone morphogenetic proteins (BMPs) (Piek E et al., FASEB J, 1999; 13:2105-2124). Members of the TGF-β superfamily mediate cellular responses via type I and II serine/threonine kinase receptors and their downstream nuclear effectors, referred to as Smads (Heldin C-H et al., Nature, 1997; 390:465-471). In endothelial cells, TGF-β has been shown to activate two type I receptor pathways: the activin receptor-like kinases ALK5 and ALK1. Activation of ALK1 promotes Smad1/5 phosphorylation and stimulates cell proliferation and migration. In contrast, activation of ALK5 induces Smad2/3 phosphorylation and inhibits cellular proliferation and migration (Goumans M-J et al., EMBO J. 2002; 21:1743-1753). Thus, in quiescent endothelial cells, ALK5 is the predominant mediator of TGF-13 signaling. However, during angiogenesis, ALK1 is preferentially activated (Lebrin F, Deckers M, Bertolino P, ten Dijke P. Cardiovasc Res 2005; 65:599-608).

Mutations in CD105 lead to hereditary hemorrhagic telangiectasia type I (or Osler-Rendu-Weber syndrome 1) (Bobik A. Arterioscler Thromb Vasc Biol 2006; 26:1712-20). This syndrome is an inherited autosomal-dominant disorder and is characterized by multisystemic vascular dysplasias, recurrent episodes of epistaxis, mucocutaneous telangiectases, and arteriovenous malformations of the lung, brain, liver, and gastrointestinal tract (Bertolino P, Deckers M, Lebrin F, ten Dijke P. Chest 2005; 128:585-905; Bobik A. Arterioscler Thromb Vasc Biol 2006; 26:1712-20). Two genetic forms of the disease have been described: hereditary hemorrhagic telangiectasia 1, characterized by a mutation in CD105, and hereditary hemorrhagic telangiectasia 2, characterized by a mutation in ALK1 (Bobik A. Arterioscler Thromb Vasc Biol 2006; 26:1712-20; Lebrin F, Deckers M, Bertolino P, ten Dijke P. Cardiovasc Res 2005; 65:599-608). In a transgenic rodent model of hereditary hemorrhagic telangiectasia with a heterozygous genotype for CD105 (CD105^{+/-}), mice exhibit irregular, dilated, and thinner-walled vessels with fewer associated vascular smooth muscle cells than wild-type animals. Interestingly, CD105 heterozygous mice survive to adulthood, while mice displaying the homozygous null mutation (CD105^{-/-}) fail to develop, leading to embryonic lethality by day E11.5 due to defective yolk sac vascularization, heart valve abnormalities, and irregular ventricular development (Arthur H M, Ure J, Smith A J, et al., Dev Biol 2000; 217:42-53; Li D Y, Sorensen L K, Brooke B S, et al. Science 1999; 284:1534-7). Thus, the above findings underscore the importance of CD105 in vascular homeostasis. Recently, CD105 has also been implicated in modulating endothelial cell migration and cytoskeletal organization (Conley B A et al., J Biol Chem 2004; 279:27440-27449; Sanz-Rodriguez F et al., J Biol Chem, 2004; 279: 32858-32868).

CD105 expression has been reported to be associated with poor prognosis in cancer patients. More specifically, CD105 expression was correlated with poor overall survival in patients with breast, lung, and colorectal cancer (Kumar S et al., Cancer Res 1999; 59:856-861; Tanaka F et al., Clin Cancer Res 2001; 7:3410-3415; Li C et al., Br J Cancer 2003; 88:1424-1431). Also, in gastrointestinal, breast, as described above, prostate, and head and neck malignancies, CD105 expression was associated with the presence of distant metastatic disease (Ding S, Li C, Lin S, et al. Hum Pathol 2006; 37:861-6; Saad R S, El-Gohary Y, Memari E, Liu Y L, Silverman J F. Hum Pathol 2005; 36:955-61; Saad R S, Liu Y L, Nathan G, Celebrezze J, Medich D, Silverman J F. Mod Pathol 2004; 17:197-203; Li C, Guo B, Wilson P B, et al. Int J Cancer 2000; 89:122-6; Yang L Y, Lu W Q, Huang G W, Wang W. BMC Cancer 2006; 6:110; El-Gohary Y M, Silverman J F, Olson P R, et al. Am J Clin Pathol 2007; 127:572-9; Chien C Y, Su C Y, Hwang C F, Chuang H C, Chen C M, Huang C C. J Surg Oncol 2006; 94:413-7).

Recently, increased levels of CD105 expression have been reported following inhibition of the VEGF pathway. In a pancreatic carcinoma xenograft model, CD105 transcript levels were upregulated more than two-fold in mice treated with an anti-VEGF neutralizing antibody (Bockhorn M et al., Clin Cancer Res. 2003; 9:4221-4226). In a bladder carcinoma xenograft model, CD105 levels, as determined by immunohistochemistry, were elevated within the tumor core in mice treated with an anti-VEGF neutralizing antibody (Davis D et al., Cancer Res. 2004; 64:4601-4610).

In addition, CD105 expression is increased by hypoxia and has been reported to protect hypoxic cells from apoptosis; suppression of CD105 increased cell apoptosis under hypoxic stress (Li C, Issa R, Kumar P, et al. J Cell Sci 2003; 116:2677-85). CD105 mRNA and promoter activity were also markedly elevated under hypoxic conditions. (Li C, Issa R, Kumar P, et al. J Cell Sci 2003; 116:2677-85). Thus, hypoxia is thought to be a potent stimulus for CD105 gene expression in vascular endothelial cells.

Thus there is a need to identify new means of inhibiting CD105 signaling.

### SUMMARY OF THE INVENTION

The present invention relates to targeted binding agents that specifically bind to CD105 and inhibit the biological activity of CD105. Embodiments of the invention relate to targeted binding agents that specifically bind to CD105 and inhibit CD105 dependent TGF-beta signaling. For example, the CD105 binding agents of the invention inhibit binding of a CD105 ligand, such as TGF-beta 1, TGF-beta 3, activin-A, BMP-2, and/or BMP-7, to the CD105 portion of the TGF-beta 1 receptor complex.

Embodiments of the invention relate to targeted binding agents that specifically bind to CD105 and inhibit binding of a CD105 ligand to CD105. In one embodiment of the invention the targeted binding agent specifically binds to CD105 and inhibits binding of the CD105 ligand of TGF-beta 1, TGF-beta 3, activin-A, BMP-2, and/or BMP-7, to CD105. In one embodiment the targeted binding agent inhibits at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% of binding of a CD105 ligand to CD105 compared to binding that would occur in the absence of the targeted binding agent.

In some embodiments of the invention, the targeted binding agent binds CD105 with a binding affinity (K_{D}) of less than 5 nanomolar (nM). In other embodiments, the targeted binding agent binds with a K_{D} of less than 4 nM, 3 nM, 2 nM or 1 nM. In some embodiments of the invention, the targeted binding agent binds CD105 with a K_{D} of less than 950 picomolar (pM). In some embodiments of the invention, the targeted binding agent binds CD105 with a K_{D} of less than 900 pM. In other embodiments, the targeted binding agent binds with a K_{D} of less than 800 pM, 700 pM or 600 pM. In some embodiments of the invention, the targeted binding agent binds CD105 with a K_{D} of less than 500 pM. In other embodiments, the targeted binding agent binds with a K_{D} of less than 400 pM. In still other embodiments, the targeted binding agent binds with a K_{D} of less than 300 pM. In some other embodiments, the targeted binding agent binds with a K_{D} of less than 200 pM. In some other embodiments, the targeted binding agent binds with a K_{D} of less than 100 pM. In one specific embodiment, the targeted binding agent of the invention can bind human CD105 with an affinity K_{D} of less than 10 pM. In another specific embodiment, the targeted binding agent of the invention can bind human CD105 with an affinity K_{D} of less than 1 pM. The K_{D} may be assessed using a method described herein or known to one of skill in the art (e.g., a BIAcore assay, ELISA, FACS) (Biacore International AB, Uppsala, Sweden).

The binding properties of the targeted binding agent or antibody of the invention may also be measured by reference to the dissociation or association rates (k_{off} and kₒₙ respectively).

In one embodiment of the invention, a targeted binding agent or an antibody may have an kₒₙ rate (antibody (Ab)+antigen (Ag)^{k}^{ₒₙ}→Ab-Ag) of at least 10⁴ M⁻¹s⁻¹, at least 5×10⁴M⁻¹s⁻¹, at least 10⁵M⁻¹s⁻¹, at least 2×10⁵M⁻¹s⁻¹, at least 5×10⁵M⁻¹s⁻¹, at least 10⁶M⁻¹s⁻¹, at least 5×10⁶M⁻¹s⁻¹, at least 10⁷M⁻¹s⁻¹, at least 5×10⁷M⁻¹s⁻¹, or at least 10⁸M⁻¹s⁻¹.

In another embodiment of the invention, targeted binding agent or an antibody may have a k_{off} rate ((Ab-Ag)^{k}^{_{off}}→antibody (Ab)+antigen (Ag)) of less than 5×10⁻¹s⁻¹, less than 10⁻¹s⁻¹, less than 5×10⁻² s⁻¹, less than 10⁻² s⁻¹, less than 5×10⁻³ s⁻¹, less than 10⁻³ s⁻¹, less than 5×10⁻⁴ s⁻¹, less than 10⁻⁴ s⁻¹, less than 5×10⁻⁵ s⁻¹, less than 10⁻⁵s⁻¹, less than 5×10⁻⁶ s⁻¹, less than 10⁻⁶s⁻¹, less than 5×10⁻⁷ s⁻¹, less than 10⁻⁷s⁻¹, less than 5×10⁻⁸ s⁻¹, less than 10⁻⁸s⁻¹, less than 5×10⁻⁹ s⁻¹, less than 10⁻⁹ s⁻¹, or less than 10⁻¹⁰s⁻¹.

In some examples the targeted binding agent of the invention is cross-reactive with other CD105 proteins from other species. In one embodiment, the targeted binding agent, e.g., 4.120, 6B1, 9H10, 10C9, 4D4, 11H2, 4.37, 6B10, 3C1, and 6A6, of the invention is cross-reactive with cynomolgus monkey CD105. In another embodiment, the targeted binding agent of the invention is cross-reactive with mouse CD105, e.g., 6B1.

The targeted binding agents of the invention can also have anti-proliferative activity. In a specific example, the antibodies of the invention can inhibit proliferation by at least 5%, 6%, 7%, 8%, 9%, 10%, 11%, 12% 13%, 14%, 15%, 20%, 25%, 30%, 40%, 50%, 60%, 70%, 80%, 90% or more. In one embodiment, the antibodies of the invention can inhibit proliferation of HUVEC cells in the range between 2-30%, 4-25%, or 8-20% when the antibody concentration is 50 μg/ml.

In another embodiment of the invention, the targeted binding agent of the invention can modulate vessel formation. In one example, the antibodies of the invention can inhibit vessel lengthening and/or the number of bifurcations. In one specific embodiment, the antibodies of the invention can inhibit vessel lengthening by at least 5%, 10%, 15%, 20%, 25%, 30%, 40%, 50%, 60%, 70%, 80%, 90% or more. For example, antibody 6B10 can inhibit vessel lengthening by at least 20%, e.g., between 20-30% and the number of bifurcations by at least 40%, e.g., between 40-60% in a whole-well image analysis method as described in Example 6.

In another embodiment of the invention the antibodies of the invention can modulate the actin cytoskeleton structure of cells. In one specific example, the targeted antibody of 3C.1, 6B1, 6B10, 10C9, 4.120 or 4.37 can cause pronounced modulation of the actin cytoskeleton structure of endothelial cells.

In another embodiment of the invention, the targeted binding agent of the invention disrupts TGFβ signaling. In one embodiment, the targeted binding agent of the invention, e.g., 4D4, 6A6, 6B10, 9H10, 4.120 or 4.37, mediates pSMAD2 phosphorylation.

In another embodiment of the invention, the targeted binding agent cross-competes with SN6 antibody, e.g., 6A6, 6B10, 9H10 or 3C1.

In some embodiments, the targeted binding agent can treat a condition associated with angiogenesis. In one embodiment of the invention, the targeted binding agent inhibits tumour growth and/or metastasis in a mammal. In particular, the targeted binding agent can be used to treat solid tumors. The targeted binding agents can be used in combination with other anti-cancer therapies such as chemotherapy regimes, or alone to inhibit tumor growth and/or metastasis. When used as a monotherapy, the targeted binding agents of the invention can be used on patients who have failed other therapies such as anti-VEGF therapies. In another embodiment, the targeted binding agent can treat ocular diseases such as diabetic retinopathy and macular degeneration due to neovascularization. In yet another embodiment, the targeted binding agent can be used to treat chronic inflammatory diseases such as rheumatoid arthritis, osteoarthritis, asthma, Crohn's disease, ulcerative colitis and inflammatory bowel disease.

In some embodiments of the invention, the targeted binding agent is an antibody. In some embodiments of the invention, the targeted binding agent is a monoclonal antibody. In one embodiment of the invention, the targeted binding agent is a fully human monoclonal antibody. In another embodiment of the invention, the targeted binding agent is a fully human monoclonal antibody of the IgG1, IgG2, IgG3 or IgG4 isotype. In another embodiment of the invention, the targeted binding agent is a fully human monoclonal antibody of the IgG2 isotype. This isotype has reduced potential to elicit effector function in comparison with other isotypes, which may lead to reduced toxicity. In another embodiment of the invention, the targeted binding agent is a fully human monoclonal antibody of the IgG1 isotype. The IgG1 isotype has increased potential to elicit ADCC and/or CDC in comparison with other isotypes, which may lead to improved efficacy. The IgG1 isotype has improved stability in comparison with other isotypes, e.g. IgG4, which may lead to improved bioavailability, or improved ease of manufacture or a longer half-life. In one embodiment, the fully human monoclonal antibody of the IgG1 isotype is of the z, za or f allotype.

In one embodiment of the invention, the targeted binding agent that specifically binds to CD105 can exhibit one or more of the following properties including:
binds human CD105 with a K_{D} of less than 1 nM;
inhibits cell proliferation of HUVEC cells by at greater than 5%, e.g., between 5-20%;
increases SMAD2 phosphorylation;
exhibits anti-angiogenic activity; and
exhibits ADCC activity.

A further embodiment is a targeted binding agent or an antibody that specifically binds to CD105 and comprises a sequence comprising one of the complementarity determining regions (CDR) sequences shown in Table 2. Embodiments of the invention include a targeted binding agent or antibody comprising a sequence comprising: any one of a CDR1, a CDR2 or a CDR3 sequence from a heavy chain variable domain as shown in Table 2. A further embodiment is a targeted binding agent or an antibody that specifically binds to CD105 and comprises a sequence comprising two of the CDR sequences of a heavy chain variable domain as shown in Table 2. In another embodiment the targeted binding agent or antibody comprises a sequence comprising a CDR1, a CDR2 and a CDR3 sequence of a heavy chain variable domain as shown in Table 2. In another embodiment the targeted binding agent or antibody comprises a sequence comprising one of the CDR sequences of a light chain variable domain as shown in Table 2. Embodiments of the invention include a targeted binding agent or antibody comprising a sequence comprising: any one of a CDR1, a CDR2 or a CDR3 sequence of a heavy chain variable domain as shown in Table 2. In another embodiment the targeted binding agent or antibody comprises a sequence comprising two of the CDR sequences of a heavy chain variable domain as shown in Table 2. In another embodiment the targeted binding agent or antibody comprises a sequence comprising a CDR1, a CDR2 and a CDR3 sequence of a light chain variable domain as shown as shown in Table 2. In another embodiment the targeted binding agent or antibody may comprise a sequence comprising a CDR1, a CDR2 and a CDR3 sequence of a heavy chain variable domain as shown as shown in Table 2 and a CDR1, a CDR2 and a CDR3 sequence of a light chain variable domain as shown in Table 2. In some embodiments, the targeted binding agent is an antibody. In certain embodiments, the targeted binding agent is a fully human monoclonal antibody. In certain other embodiments, the targeted binding agent is a binding fragment of a fully human monoclonal antibody.

In one embodiment, the antibody of the invention includes:
(a) a VH CDR1 of SEQ ID NO:2 having an amino acid sequence identical to or comprising 1, 2, or 3 amino acid residue substitutions relative to the VH CDR1 of SEQ ID NO:2;
(b) a VH CDR2 of SEQ ID NO:2 having an amino acid sequence identical to or comprising 1, 2, or 3 amino acid residue substitutions relative to the VH CDR2 of SEQ ID NO:2;
(c) a VH CDR3 of SEQ ID NO:2 having an amino acid sequence identical to or comprising 1, 2, or 3 amino acid residue substitutions relative to the VH CDR3 of SEQ ID NO:2;
(d) a VL CDR1 of SEQ ID NO:4 having an amino acid sequence identical to or comprising 1, 2, or 3 amino acid residue substitutions relative to VL CDR1 of SEQ ID NO:4;
(e) a VL CDR2 of SEQ ID NO:4 having an amino acid sequence identical to or comprising 1, 2, or 3 amino acid residue substitutions relative to the VL CDR2 of SEQ ID NO:4; and
(f) a VL CDR3 of SEQ ID NO:4 having an amino acid sequence identical to or comprising 1, 2, or 3 amino acid residue substitutions relative to the VL CDR3 of SEQ ID NO:4.

In another embodiment, the antibody of the invention includes:
(a) a VH CDR1 of SEQ ID NO:26 having an amino acid sequence identical to or comprising 1, 2, or 3 amino acid residue substitutions relative to the VH CDR1 of SEQ ID NO:26;
(b) a VH CDR2 of SEQ ID NO:26 having an amino acid sequence identical to or comprising 1, 2, or 3 amino acid residue substitutions relative to the VH CDR2 of SEQ ID NO:26;
(c) a VH CDR3 of SEQ ID NO:26 having an amino acid sequence identical to or comprising 1, 2, or 3 amino acid residue substitutions relative to the VH CDR3 of SEQ ID NO:26;
(d) a VL CDR1 of SEQ ID NO:28 having an amino acid sequence identical to or comprising 1, 2, or 3 amino acid residue substitutions relative to VL CDR1 of SEQ ID NO:28;
(e) a VL CDR2 of SEQ ID NO:28 having an amino acid sequence identical to or comprising 1, 2, or 3 amino acid residue substitutions relative to the VL CDR2 of SEQ ID NO:28; and
(f) a VL CDR3 of SEQ ID NO:28 having an amino acid sequence identical to or comprising 1, 2, or 3 amino acid residue substitutions relative to the VL CDR3 of SEQ ID NO:28.

In yet another embodiment, the invention includes an antibody including:
(a) a VH CDR1 of SEQ ID NO:30 having an amino acid sequence identical to or comprising 1, 2, or 3 amino acid residue substitutions relative to the VH CDR1 of SEQ ID NO:30;
(b) a VH CDR2 of SEQ ID NO:30 having an amino acid sequence identical to or comprising 1, 2, or 3 amino acid residue substitutions relative to the VH CDR2 of SEQ ID NO:30;
(c) a VH CDR3 of SEQ ID NO:30 having an amino acid sequence identical to or comprising 1, 2, or 3 amino acid residue substitutions relative to the VH CDR3 of SEQ ID NO:30;
(d) a VL CDR1 of SEQ ID NO:32 having an amino acid sequence identical to or comprising 1, 2, or 3 amino acid residue substitutions relative to VL CDR1 of SEQ ID NO:32;
(e) a VL CDR2 of SEQ ID NO:32 having an amino acid sequence identical to or comprising 1, 2, or 3 amino acid residue substitutions relative to the VL CDR2 of SEQ ID NO:32; and
(f) a VL CDR3 of SEQ ID NO:32 having an amino acid sequence identical to or comprising 1, 2, or 3 amino acid residue substitutions relative to the VL CDR3 of SEQ ID NO:32.

In another embodiment the targeted binding agent may comprise a sequence comprising any one of the CDR1, CDR2 or CDR3 of the variable heavy chain sequences encoded by a polynucleotide in a plasmid designated Mab4.120VH, Mab4.37VH, or Mab6B10VH which were deposited at the American Type Culture Collection (ATCC) under number PTA-9514, PTA-9511, or PTA-9510 on Sep. 17, 2008. In another embodiment the targeted binding agent may comprise a sequence comprising any one of the CDR1, CDR2 or CDR3 of the variable light chain sequences encoded by a polynucleotide in a plasmid designated Mab4.120VL, Mab4.37VL, or Mab6B10VL which were deposited at the American Type Culture Collection (ATCC) under number PTA-9513, PTA-9512, or PTA-9499 on Sep. 17, 2008.

In one embodiment, a targeted binding agent or an antibody of the invention comprises a variable heavy chain amino acid sequence comprising a CDR3 encoded by the polynucleotide in plasmid designated Mab4.120VH which was deposited at the American Type Culture Collection (ATCC) under number PTA-9514 on Sep. 17, 2008.

In one embodiment, a targeted binding agent or an antibody of the invention comprises a variable heavy chain amino acid sequence comprising a CDR3 encoded by the polynucleotide in plasmid designated Mab4.120VH which was deposited at the American Type Culture Collection (ATCC) under number PTA-9514 on Sep. 17, 2008 and a variable light chain amino acid sequence comprising a CDR3 encoded by the polynucleotide in plasmid designated Mab4.120VL which was deposited at the American Type Culture Collection (ATCC) under number PTA-9513 on Sep. 17, 2008.

In another embodiment, a targeted binding agent or an antibody of the invention comprises a variable heavy chain amino acid sequence comprising at least one, at least two, or at least three of the CDRs of the antibody encoded by the polynucleotide in plasmid designated Mab4.120VH which was deposited at the American Type Culture Collection (ATCC) under number PTA-9514 on Sep. 17, 2008.

In another embodiment, a targeted binding agent or an antibody of the invention comprises a variable light chain amino acid sequence comprising at least one, at least two, or at least three of the CDRs of the antibody encoded by the polynucleotide in plasmid designated Mab4.120VL which was deposited at the American Type Culture Collection (ATCC) under number PTA-9514 on Sep. 17, 2008.

In another embodiment, a targeted binding agent or an antibody of the invention comprises a variable heavy chain amino acid sequence comprising at least one, at least two, or at least three of the CDRs of the antibody encoded by the polynucleotide in plasmid designated Mab4.120VH which was deposited at the American Type Culture Collection (ATCC) under number PTA-9514 on Sep. 17, 2008 and a variable light chain amino acid sequence comprising at least one, at least two, or at least three of the CDRs of the antibody encoded by the polynucleotide in plasmid designated Mab4.120VL which was deposited at the American Type Culture Collection (ATCC) under number PTA-9513 on Sep. 17, 2008.

In one embodiment, a targeted binding agent or an antibody of the invention comprises a variable heavy chain amino acid sequence comprising a CDR3 encoded by the polynucleotide in plasmid designated Mab4.37VH which was deposited at the American Type Culture Collection (ATCC) under number PTA-9511 on Sep. 17, 2008.

In one embodiment, a targeted binding agent or an antibody of the invention comprises a variable heavy chain amino acid sequence comprising a CDR3 encoded by the polynucleotide in plasmid designated Mab4.37VH which was deposited at the American Type Culture Collection (ATCC) under number PTA-9511 on Sep. 17, 2008 and a variable light chain amino acid sequence comprising a CDR3 encoded by the polynucleotide in plasmid designated Mab4.37VL which was deposited at the American Type Culture Collection (ATCC) under number PTA-9512 on Sep. 17, 2008.

In another embodiment, a targeted binding agent or an antibody of the invention comprises a variable heavy chain amino acid sequence comprising at least one, at least two, or at least three of the CDRs of the antibody encoded by the polynucleotide in plasmid designated Mab4.37VH which was deposited at the American Type Culture Collection (ATCC) under number PTA-9511 on Sep. 17, 2008.

In another embodiment, a targeted binding agent or an antibody of the invention comprises a variable light chain amino acid sequence comprising at least one, at least two, or at least three of the CDRs of the antibody encoded by the polynucleotide in plasmid designated Mab4.37VL which was deposited at the American Type Culture Collection (ATCC) under number PTA-9512 on Sep. 17, 2008.

In another embodiment, a targeted binding agent or an antibody of the invention comprises a variable heavy chain amino acid sequence comprising at least one, at least two, or at least three of the CDRs of the antibody encoded by the polynucleotide in plasmid designated Mab4.37VH which was deposited at the American Type Culture Collection (ATCC) under number PTA-9511 on Sep. 17, 2008 and a variable light chain amino acid sequence comprising at least one, at least two, or at least three of the CDRs of the antibody encoded by the polynucleotide in plasmid designated Mab4.37VL which was deposited at the American Type Culture Collection (ATCC) under number PTA-9512 on Sep. 17, 2008.

In one embodiment, a targeted binding agent or an antibody of the invention comprises a variable heavy chain amino acid sequence comprising a CDR3 encoded by the polynucleotide in plasmid designated Mab6B10VH which was deposited at the American Type Culture Collection (ATCC) under number PTA-9510 on Sep. 17, 2008.

In one embodiment, a targeted binding agent or an antibody of the invention comprises a variable heavy chain amino acid sequence comprising a CDR3 encoded by the polynucleotide in plasmid designated Mab6B10VH which was deposited at the American Type Culture Collection (ATCC) under number PTA-9510 on Sep. 17, 2008 and a variable light chain amino acid sequence comprising a CDR3 encoded by the polynucleotide in plasmid designated Mab6B10VL which was deposited at the American Type Culture Collection (ATCC) under number PTA-9499 on Sep. 17, 2008.

In another embodiment, a targeted binding agent or an antibody of the invention comprises a variable heavy chain amino acid sequence comprising at least one, at least two, or at least three of the CDRs of the antibody encoded by the polynucleotide in plasmid designated Mab6B10VH which was deposited at the American Type Culture Collection (ATCC) under number PTA-9510 on Sep. 17, 2008.

In another embodiment, a targeted binding agent or an antibody of the invention comprises a variable light chain amino acid sequence comprising at least one, at least two, or at least three of the CDRs of the antibody encoded by the polynucleotide in plasmid designated Mab6B10VL which was deposited at the American Type Culture Collection (ATCC) under number PTA-9499 on Sep. 17, 2008.

In another embodiment, a targeted binding agent or an antibody of the invention comprises a variable heavy chain amino acid sequence comprising at least one, at least two, or at least three of the CDRs of the antibody encoded by the polynucleotide in plasmid designated Mab6B10VH which was deposited at the American Type Culture Collection (ATCC) under number PTA-9510 on Sep. 17, 2008 and a variable light chain amino acid sequence comprising at least one, at least two, or at least three of the CDRs of the antibody encoded by the polynucleotide in plasmid designated Mab6B10VL which was deposited at the American Type Culture Collection (ATCC) under number PTA-9499 on Sep. 17, 2008.

In another embodiment, a targeted binding agent or an antibody of the invention comprises a variable heavy chain of an antibody encoded by the polynucleotide in plasmid designated Mab4.120VH which was deposited at the American Type Culture Collection (ATCC) under number PTA-9514 on Sep. 17, 2008.

In another embodiment, a targeted binding agent or an antibody of the invention comprises a variable heavy chain of an antibody encoded by the polynucleotide in plasmid designated Mab4.37VH which was deposited at the American Type Culture Collection (ATCC) under number PTA-9511 on Sep. 17, 2008.

In another embodiment, a targeted binding agent or an antibody of the invention comprises a variable heavy chain of an antibody encoded by the polynucleotide in plasmid designated Mab6B10VH which was deposited at the American Type Culture Collection (ATCC) under number PTA-9510 on Sep. 17, 2008.

In another embodiment, a targeted binding agent or an antibody of the invention comprises a variable light chain of an antibody encoded by the polynucleotide in plasmid designated Mab4.120VL which was deposited at the American Type Culture Collection (ATCC) under number PTA-9513 on Sep. 17, 2008.

In another embodiment, a targeted binding agent or an antibody of the invention comprises a variable light chain of an antibody encoded by the polynucleotide in plasmid designated Mab4.37VL which was deposited at the American Type Culture Collection (ATCC) under number PTA-9512 on Sep. 17, 2008.

In another embodiment, a targeted binding agent or an antibody of the invention comprises a variable light chain of an antibody encoded by the polynucleotide in plasmid designated Mab6B10VL which was deposited at the American Type Culture Collection (ATCC) under number PTA-9499 on Sep. 17, 2008.

In another embodiment, a targeted binding agent or an antibody of the invention comprises a variable heavy chain of an antibody encoded by the polynucleotide in plasmid designated Mab4.120VH which was deposited at the American Type Culture Collection (ATCC) under number PTA-9514 on Sep. 17, 2008 and a variable light chain of an antibody encoded by the polynucleotide in plasmid designated Mab4.120VL which was deposited at the American Type Culture Collection (ATCC) under number PTA-9513 on Sep. 17, 2008.

In another embodiment, a targeted binding agent or an antibody of the invention comprises a variable light chain of an antibody encoded by the polynucleotide in plasmid designated Mab4.37VL which was deposited at the American Type Culture Collection (ATCC) under number PTA-9512 on Sep. 17, 2008 and a variable heavy chain of an antibody encoded by the polynucleotide in plasmid designated Mab4.37VH which was deposited at the American Type Culture Collection (ATCC) under number PTA-9511 on Sep. 17, 2008.

In another embodiment, a targeted binding agent or an antibody of the invention comprises a variable heavy chain of an antibody encoded by the polynucleotide in plasmid designated Mab6B10VH which was deposited at the American Type Culture Collection (ATCC) under number PTA-9510 on Sep. 17, 2008 and a variable light chain of an antibody encoded by the polynucleotide in plasmid designated Mab6B10VL which was deposited at the American Type Culture Collection (ATCC) under number PTA-9499 on Sep. 17, 2008.

It is noted that those of ordinary skill in the art can readily accomplish CDR determinations. See for example, Kabat et al., Sequences of Proteins of Immunological Interest, Fifth Edition, NIH Publication 91-3242, Bethesda Md. (1991), vols. 1-3. Kabat provides multiple sequence alignments of immunoglobulin chains from numerous species antibody isotypes. The aligned sequences are numbered according to a single numbering system, the Kabat numbering system. The Kabat sequences have been updated since the 1991 publication and are available as an electronic sequence database (latest downloadable version 1997). Any immunoglobulin sequence can be numbered according to Kabat by performing an alignment with the Kabat reference sequence. Accordingly, the Kabat numbering system provides a uniform system for numbering immunoglobulin chains.

In one embodiment, the targeted binding agent or antibody comprises a sequence comprising any one of the heavy chain sequences shown in Table 2. In another embodiment, the targeted binding agent or antibody comprises a sequence comprising any one of the heavy chain sequences of antibodies 4.120, 4.37 and 6B10.

Light-chain promiscuity is well established in the art, thus, a targeted binding agent or antibody comprising a sequence comprising any one of the heavy chain sequences of antibodies 4.120, 4.37 and 6B10 or another antibody as disclosed herein, may further comprise any one of the light chain sequences shown in Table 2 or of antibodies 4.120, 4.37 and 6B10, or another antibody as disclosed herein. In some embodiments, the antibody is a fully human monoclonal antibody.

In one embodiment, the targeted binding agent or antibody comprises a sequence comprising any one of the light chain sequences shown in Table 2. In another embodiment, the targeted binding agent or antibody comprises a sequence comprising any one of the light chain sequences of antibodies 4.120, 4.37 and 6B10. In some embodiments, the antibody is a fully human monoclonal antibody.

In some embodiments, the targeting binding agent is a monoclonal antibody selected from the group consisting of: 4.120, 9H10, 10C9, 4D4, 11H2, 6B1, 4.37, 6B10, 3C1, and 6A6. In one embodiment, the targeted binding agent comprises one or more of fully human monoclonal antibodies 4.120, 9H10, 10C9, 4D4, 11H2, 6B1, 4.37, 6B10, 3C1, and 6A6. In certain embodiments, the targeting binding agent is monoclonal antibody 4.120. In certain other embodiments, the targeting binding agent is monoclonal antibody 4.37. In certain other embodiments, the targeting binding agent is monoclonal antibody 6B10

In one embodiment a targeted binding agent or an antibody may comprise a sequence comprising a heavy chain CDR1, CDR2 and CDR3 selected from any one of the sequences shown in Table 2. In one embodiment a targeted binding agent or an antibody may comprise a sequence comprising a light chain CDR1, CDR2 and CDR3 selected from any one of the sequences shown in Table 2. In one embodiment a targeted binding agent or an antibody may comprise a sequence comprising a heavy chain CDR1, CDR2 and CDR3 selected from any one of the CDRs of antibodies 4.120, 9H10, 10C9, 4D4, 11H2, 6B1, 4.37, 6B10, 3C1, and 6A6. In one embodiment a targeted binding agent or an antibody may comprise a sequence comprising a light chain CDR1, CDR2 and CDR3 selected from any one of the CDRs of antibodies 4.120, 9H10, 10C9, 4D4, 11H2, 6B1, 4.37, 6B10, 3C1, and 6A6.

In another embodiment the targeted binding agent or antibody may comprise a sequence comprising any one of a CDR1, a CDR2 or a CDR3 of any one of the fully human monoclonal antibodies 4.120, 4.37, or 6B10, as shown in Table 2. In another embodiment the targeted binding agent or antibody may comprise a sequence comprising any one of a CDR1, a CDR2 or a CDR3 of any one of the fully human monoclonal antibodies 4.120, 4.37, or 6B10, as shown in Table 2. In one embodiment the targeted binding agent or antibody may comprise a sequence comprising a CDR1, a CDR2 and a CDR3 of fully human monoclonal antibody 4.120, 4.37, or 6B10, as shown in Table 2. In another embodiment the targeted binding agent or antibody may comprise a sequence comprising a CDR1, a CDR2 and a CDR3 of fully human monoclonal antibody 4.120, 4.37, or 6B10, as shown in Table 2. In another embodiment the targeted binding agent or antibody may comprise a sequence comprising a CDR1, a CDR2 and a CDR3 of fully human monoclonal antibody 4.120, 4.37, or 6B10, as shown in Table 2, and a CDR1, a CDR2 and a CDR3 sequence of fully human monoclonal antibody 4.120, 4.37, or 6B10, as shown in Table 2. In some embodiments, the antibody is a fully human monoclonal antibody.

In another embodiment the targeted binding agent or antibody comprises a sequence comprising the CDR1, CDR2 and CDR3 sequence of fully human monoclonal antibody 4.120, as shown in Table 2 and the CDR1, CDR2 and CDR3 sequence of fully human monoclonal antibody 4.120 as shown in Table 2. In another embodiment the targeted binding agent or antibody comprises a sequence comprising the CDR1, CDR2 and CDR3 sequence of fully human monoclonal antibody 4.37, as shown in Table 2 and the CDR1, CDR2 and CDR3 sequence of fully human monoclonal antibody 4.37 as shown in Table 2. In another embodiment the targeted binding agent or antibody comprises a sequence comprising the CDR1, CDR2 and CDR3 sequence of fully human monoclonal antibody 6B10 as shown in Table 2 and the CDR1, CDR2 and CDR3 sequence of fully human monoclonal antibody 6B10 as shown in Table 2. In some embodiments, the antibody is a fully human monoclonal antibody.

A further embodiment of the invention is a targeted binding agent or antibody comprising a sequence comprising the contiguous sequence spanning the framework regions and CDRs, specifically from FR1 through FR4 or CDR1 through CDR3, of any one of the sequences as shown in Table 2. In one embodiment the targeted binding agent or antibody comprises a sequence comprising the contiguous sequences spanning the framework regions and CDRs, specifically from FR1 through FR4 or CDR1 through CDR3, of any one of the sequences of monoclonal antibodies 4.120, 4.37, or 6B10, as shown in Table 2. In some embodiments, the antibody is a fully human monoclonal antibody.

In another embodiment the agent or antibody, or antigen-binding portion thereof, comprises a heavy chain polypeptide comprising the sequence of SEQ ID NO.:2. In one embodiment, the agent or antibody, or antigen-binding portion thereof, further comprises a light chain polypeptide comprising the sequence of SEQ ID NO.:4. In some embodiments, the antibody is a fully human monoclonal antibody.

One embodiment provides a targeted binding agent or antibody, or antigen-binding portion thereof, wherein the agent or antibody, or antigen-binding portion thereof, comprises a heavy chain polypeptide comprising the sequence of SEQ ID NO.:26. In one embodiment, the agent or antibody, or antigen-binding portion thereof, further comprises a light chain polypeptide comprising the sequence of SEQ ID NO.:28. In some embodiments, the antibody is a fully human monoclonal antibody.

In another embodiment the agent or antibody, or antigen-binding portion thereof, comprises a heavy chain polypeptide comprising the sequence of SEQ ID NO.:30. In another embodiment, the agent or antibody, or antigen-binding portion thereof, further comprises a light chain polypeptide comprising the sequence of SEQ ID NO.:32. In some embodiments, the antibody is a fully human monoclonal antibody.

In one embodiment the targeted binding agent or antibody comprises as many as twenty, sixteen, ten, nine or fewer, e.g. one, two, three, four or five, amino acid additions, substitutions, deletions, and/or insertions within the disclosed CDRs or heavy or light chain framework sequences. Such modifications may potentially be made at any residue within the CDRs and/or framework sequences. In some embodiments, the antibody is a fully human monoclonal antibody.

In one embodiment, the targeted binding agent or antibody comprises variants or derivatives of the CDRs disclosed herein, the contiguous sequences spanning the framework regions and CDRs (specifically from FR1 through FR4 or CDR1 through CDR3), the light or heavy chain sequences disclosed herein, or the antibodies disclosed herein. Variants include targeted binding agents or antibodies comprising sequences which have as many as twenty, sixteen, ten, nine or fewer, e.g. one, two, three, four, five or six amino acid additions, substitutions, e.g., conservative amino acid substitutions, deletions, and/or insertions in any of the CDR1, CDR2 or CDR3s as shown in Table 2, the contiguous sequences spanning the framework regions and CDRs (specifically from FR1 through FR4 or CDR1 through CDR3) as shown in Table 2, the light or heavy chain sequences disclosed herein, or with the monoclonal antibodies disclosed herein. Variants include targeted binding agents or antibodies comprising sequences which have at least about 60, 70, 80, 85, 90, 95, 98 or about 99% amino acid sequence identity with any of the CDR1, CDR2 or CDR3s as shown in Table 2, the contiguous sequences spanning the framework regions and CDRs (specifically from FR1 through FR4 or CDR1 through CDR3) as shown in Table 2, the light or heavy chain sequences disclosed herein, or with the monoclonal antibodies disclosed herein. The percent identity of two amino acid sequences can be determined by any method known to one skilled in the art, including, but not limited to, pairwise protein alignment. In one embodiment variants comprise changes in the CDR sequences or light or heavy chain polypeptides disclosed herein that are naturally occurring or are introduced by in vitro engineering of native sequences using recombinant DNA techniques or mutagenesis techniques. Naturally occurring variants include those which are generated in vivo in the corresponding germline nucleotide sequences during the generation of an antibody to a foreign antigen. In one embodiment the derivative may be a heteroantibody, that is an antibody in which two or more antibodies are linked together. Derivatives include antibodies which have been chemically modified. Examples include covalent attachment of one or more polymers, such as water-soluble polymers, N-linked, or O-linked carbohydrates, sugars, phosphates, and/or other such molecules. The derivatives are modified in a manner that is different from the naturally occurring or starting antibody, either in the type or location of the molecules attached. Derivatives further include deletion of one or more chemical groups which are naturally present on the antibody.

In one embodiment, the targeted binding agent is a bispecific antibody. A bispecific antibody is an antibody that has binding specificity for at least two different epitopes. Methods for making bispecific antibodies are known in the art. (See, for example, Millstein et al., Nature, 305:537-539 (1983); Traunecker et al., EMBO J., 10:3655-3659 (1991); Suresh et al., Methods in Enzymology, 121:210 (1986); Kostelny et al., J. Immunol., 148(5):1547-1553 (1992); Hollinger et al., Proc. Natl Acad. Sci. USA, 90:6444-6448 (1993); Gruber et al., J. Immunol., 152:5368 (1994); U.S. Pat. Nos. 4,474,893; 4,714,681; 4,925,648; 5,573,920; 5,601,81; 95,731,168; 4,676,980; and 4,676,980, WO 94/04690; WO 91/00360; WO 92/200373; WO 93/17715; WO 92/08802; and EP 03089.) In one example, a bispecific antibody of the present invention is an antibody that has binding specificity for at least two different CD105 epitopes. Since a number of the CD105 targeted binding agents of the invention have different epitopes or have partial or overlapping epitopes it is contemplated that a bispecific antibody of the invention can include any combination of the CD105 targeted binding agents having different or overlapping epitopes. For example, 6A6 and 6B10 have a different epitope from 4D4 and 10C9. In one example the bispecific antibody has the variable or hypervariable region of 6A6 or 6B10 and variable or hypervariable region of 4D4 or 10C9.

In some embodiments of the invention, the targeted binding agent or antibody comprises a sequence comprising SEQ ID NO.: 26. In certain embodiments, SEQ ID NO.: 26 comprises any one of the combinations of germline and non-germline residues indicated by each row of Table 5. In some embodiments, SEQ ID NO: 26 comprises any one, any two, or all two of the germline residues as indicated in Table 5. In certain embodiments, SEQ ID NO.: 2 comprises any one of the unique combinations of germline and non-germline residues indicated by each row of Table 5. In other embodiments, the targeted binding agent or antibody is derived from a germline sequence with VH3-33, D6-13 and JH6, domains, wherein one or more residues has been mutated to yield the corresponding germline residue at that position.

A further embodiment of the invention is a targeted binding agent or antibody which competes for binding to CD105 with the targeted binding agent or antibodies of the invention. In another embodiment of the invention there is an antibody which competes for binding to CD105 with the targeted binding agent or antibodies of the invention. In another embodiment the targeted binding agent or antibody competes for binding to CD105 with any one of fully human monoclonal antibodies 4.120, 9H10, 10C9, 4D4, 11H2, 6B1, 4.37, 6B10, 3C1, or 6A6. “Competes” indicates that the targeted binding agent or antibody competes for binding to CD105 with any one of fully human monoclonal antibodies 4.120, 9H10, 10C9, 4D4, 11H2, 6B1, 4.37, 6B10, 3C1, or 6A6, i.e. competition is unidirectional.

Embodiments of the invention include a targeted binding agent or antibody which cross competes with any one of fully human monoclonal antibodies 4.120, 9H10, 10C9, 4D4, 11H2, 6B1, 4.37, 6B10, 3C1, or 6A6 for binding to CD105. “Cross competes” indicates that the targeted binding agent or antibody competes for binding to CD105 with any one of fully human monoclonal antibodies 4.120, 9H10, 10C9, 4D4, 11H2, 6B1, 4.37, 6B10, 3C1, or 6A6, and vice versa, i.e. competition is bidirectional.

A further embodiment of the invention is a targeted binding agent or antibody which competes for binding to CD105. In another embodiment of the invention there is a targeted binding agent or antibody which cross-competes with the targeted binding agent or antibodies of the invention for binding to CD105.

A further embodiment of the invention is a targeted binding agent or antibody that binds to the same epitope on CD105 as the targeted binding agent or antibodies of the invention. Embodiments of the invention also include a targeted binding agent or antibody that binds to the same epitope on CD105 as any one of fully human monoclonal antibodies 4.120, 9H10, 10C9, 4D4, 11H2, 6B1, 4.37, 6B10, 3C1, or 6A6.

Other embodiments of the invention include isolated nucleic acid molecules encoding any of the targeted binding agents or antibodies described herein, vectors having isolated nucleic acid molecules encoding the targeted binding agents or antibodies described herein or a host cell transformed with any of such nucleic acid molecules. Embodiments of the invention include a nucleic acid molecule encoding a fully human isolated targeted binding agent that specifically bind to CD105 and inhibit binding of a CD105 ligand such as TGF-13 to the CD105 receptor. The invention also encompasses polynucleotides that hybridize under stringent or lower stringency hybridization conditions, as defined herein, to polynucleotides that encode any of the targeted binding agents or antibodies described herein. Embodiments of the invention also include a vector comprising the nucleic acid molecule encoding the binding agent. Additional embodiments include a host cell comprising the vector of comprising the nucleic acid molecule.

As known in the art, antibodies can advantageously be, for example, polyclonal, oligoclonal, monoclonal, chimeric, humanised, and/or fully human antibodies.

It will be appreciated that embodiments of the invention are not limited to any particular form of an antibody or method of generation or production. In some embodiments of the invention, the targeted binding agent is a binding fragment of a fully human monoclonal antibody. For example, the targeted binding agent can be a full-length antibody (e.g., having an intact human Fc region) or an antibody binding fragment (e.g., a Fab, Fab′ or F(ab′)₂, FV or dAb). In addition, the antibodies can be single-domain antibodies such as camelid or human single VH or VL domains that bind to CD105, such as a dAb fragment.

Embodiments of the invention described herein also provide cells for producing these antibodies. Examples of cells include hybridomas, or recombinantly created cells, such as Chinese hamster ovary (CHO) cells, variants of CHO cells (for example DG44) and NSO cells that produce antibodies against CD105. Additional information about variants of CHO cells can be found in Andersen and Reilly (2004) Current Opinion in Biotechnology 15, 456-462 which is incorporated herein in its entirety by reference. The antibody can be manufactured from a hybridoma that secretes the antibody, or from a recombinantly engineered cell that has been transformed or transfected with a gene or genes encoding the antibody.

In addition, one embodiment of the invention is a method of producing an antibody of the invention by culturing host cells under conditions wherein a nucleic acid molecule is expressed to produce the antibody followed by recovering the antibody. It should be realised that embodiments of the invention also include any nucleic acid molecule which encodes an antibody or fragment of an antibody of the invention including nucleic acid sequences optimised for increasing yields of antibodies or fragments thereof when transfected into host cells for antibody production.

A further embodiment herein includes a method of producing antibodies that specifically bind to CD105 and inhibit the biological activity of CD105, by immunising a mammal with cells expressing human CD105, isolated cell membranes containing human CD105, purified human CD105, or a fragment thereof, and/or one or more orthologous sequences or fragments thereof.

In other embodiments the invention provides compositions, including a targeted binding agent or antibody of the invention or binding fragment thereof, and a pharmaceutically acceptable carrier or diluent.

Still further embodiments of the invention include methods of effectively treating an animal suffering from a proliferative, angiogenic, disease by administering to the animal a therapeutically effective dose of a targeted binding agent that specifically binds to CD105. In certain embodiments the method further comprises selecting an animal in need of treatment a tumor, cancer, and/or a cell proliferative disorder, and administering to the animal a therapeutically effective dose of a targeted binding agent that specifically binds to CD105.

Still further embodiments of the invention include methods of effectively treating an animal suffering from a neoplastic disease by administering to the animal a therapeutically effective dose of a targeted binding agent that specifically binds to CD105. In certain embodiments the method further comprises selecting an animal in need of treatment for a neoplastic disease, and administering to the animal a therapeutically effective dose of a targeted binding agent that specifically binds to CD105.

Still further embodiments of the invention include methods of effectively treating an animal suffering from a malignant tumour by administering to the animal a therapeutically effective dose of a targeted binding agent that specifically binds to CD105. In certain embodiments the method further comprises selecting an animal in need of treatment for a malignant tumour, and administering to the animal a therapeutically effective dose of a targeted binding agent that specifically binds to CD105.

Still further embodiments of the invention include methods of effectively treating an animal suffering from a disease or condition associated with CD105 expression by administering to the animal a therapeutically effective dose of a targeted binding agent that specifically binds to CD105. In certain embodiments the method further comprises selecting an animal in need of treatment for a disease or condition associated with CD105 expression, and administering to the animal a therapeutically effective dose of a targeted binding agent that specifically binds to CD105.

A malignant tumour may be selected from the group consisting of: melanoma, small cell lung cancer, non-small cell lung cancer, glioma, hepatocellular (liver) carcinoma, thyroid tumour, gastric (stomach) cancer, prostate cancer, breast cancer, ovarian cancer, bladder cancer, lung cancer, glioblastoma, endometrial cancer, kidney cancer, colon cancer, pancreatic cancer, esophageal carcinoma, head and neck cancers, mesothelioma, sarcomas, biliary (cholangiocarcinoma), small bowel adenocarcinoma, pediatric malignancies and epidermoid carcinoma.

Treatable proliferative or angiogenic diseases include neoplastic diseases, such as, melanoma, small cell lung cancer, non-small cell lung cancer, glioma, advanced non-small cell lung cancer, hepatocellular (liver) carcinoma, thyroid tumour, gastric (stomach) cancer, gallbladder cancer, prostate cancer, breast cancer, ovarian cancer, bladder cancer, renal cell cancer, lung cancer, glioblastoma, endometrial cancer, kidney cancer, colon cancer, pancreatic cancer, esophageal carcinoma, head and neck cancers, mesothelioma, sarcomas, biliary (cholangiocarcinoma), small bowel adenocarcinoma, pediatric malignancies, epidermoid carcinoma and leukaemia, including chronic myelogenous leukaemia.

In one embodiment, the target binding agents of the invention can be used to treat solid tumors, including lung, breast, colorectal, prostate, ovarian, hepatocellular carcinoma, head and neck, glioblastoma, esophageal.

In one embodiment the present invention is suitable for use in inhibiting CD105, in patients with a tumour which is dependent alone, or in part, on CD105.

Still further embodiments of the invention include use of a targeted binding agent or antibody of the invention in the preparation of a medicament for the treatment of an animal suffering from a proliferative, or angiogenic related disease. In certain embodiments the use further comprises selecting an animal in need of treatment for a proliferative, or angiogenic-related disease.

Still further embodiments of the invention include use of a targeted binding agent or antibody of the invention in the preparation of a medicament for the treatment of an animal suffering from a neoplastic disease. In certain embodiments the use further comprises selecting an animal in need of treatment for a neoplastic disease.

Still further embodiments of the invention include use of a targeted binding agent or antibody of the invention in the preparation of a medicament for the treatment of an animal suffering from a non-neoplastic disease. In certain embodiments the use further comprises selecting an animal in need of treatment for a non-neoplastic disease.

Still further embodiments of the invention include use of a targeted binding agent or antibody of the invention in the preparation of a medicament for the treatment of an animal suffering from a malignant tumour. In certain embodiments the use further comprises selecting an animal in need of treatment for a malignant tumour.

Still further embodiments of the invention include use of a targeted binding agent or antibody of the invention in the preparation of a medicament for the treatment of an animal suffering from a disease or condition associated with CD105 expression. In certain embodiments the use further comprises selecting an animal in need of treatment for a disease or condition associated with CD105 expression.

Still further embodiments of the invention include a targeted binding agent or antibody of the invention for use as a medicament for the treatment of an animal suffering from a proliferative or angiogenic-related disease.

Still further embodiments of the invention include a targeted binding agent or antibody of the invention for use as a medicament for the treatment of an animal suffering from a neoplastic disease.

Still further embodiments of the invention include a targeted binding agent or antibody of the invention for use as a medicament for the treatment of an animal suffering from a malignant tumour.

Still further embodiments of the invention include a targeted binding agent or antibody of the invention for use as a medicament for the treatment of an animal suffering from a disease or condition associated with CD105 expression.

Still further embodiments of the invention include a targeted binding agent or antibody of the invention for use as a medicament for the treatment of an animal suffering from a CD105 induced disease.

In one embodiment treatment of a
a proliferative or angiogenic-related disease;
a neoplastic disease;
a malignant tumour;
an ocular disease;
a chronic inflammatory disease
a disease or condition associated with CD105 expression; or
comprises managing, ameliorating, preventing, any of the aforementioned diseases or conditions.

In one embodiment treatment of a neoplastic disease comprises inhibition of tumour growth, tumour growth delay, regression of tumour, shrinkage of tumour, increased time to regrowth of tumour on cessation of treatment, increased time to tumour recurrence, slowing of disease progression.

In some embodiments of the invention, the animal to be treated is a human.

In some embodiments of the invention, the targeted binding agent is a fully human monoclonal antibody.

In some embodiments of the invention, the targeted binding agent is selected from the group consisting of fully human monoclonal antibodies 4.120, 9H10, 10C9, 4D4, 11H2, 6B1, 4.37, 6B10, 3C1, and 6A6.

Embodiments of the invention include a conjugate comprising the targeted binding agent as described herein, and a therapeutic agent. In some embodiments of the invention, the therapeutic agent is a toxin. In other embodiments, the therapeutic agent is a radioisotope. In still other embodiments, the therapeutic agent is a pharmaceutical composition.

In another aspect, a method of selectively killing a cancerous cell in a patient is provided. The method comprises administering a fully human antibody conjugate to a patient. The fully human antibody conjugate comprises an antibody that can bind to CD105 and an agent. The agent is either a toxin, a radioisotope, or another substance that will kill a cancer cell. The antibody conjugate thereby selectively kills the cancer cell.

In one aspect, a conjugated fully human antibody that specifically binds to CD105 is provided. Attached to the antibody is an agent, and the binding of the antibody to a cell results in the delivery of the agent to the cell. In one embodiment, the above conjugated fully human antibody binds to an extracellular domain of CD105. In another embodiment, the antibody and conjugated toxin are internalised by a cell that expresses CD105. In another embodiment, the agent is a cytotoxic agent. In another embodiment, the agent is, for example saporin, or auristatin, pseudomonas exotoxin, gelonin, ricin, calicheamicin or maytansine-based immunoconjugates, and the like. In still another embodiment, the agent is a radioisotope.

The targeted binding agent or antibody of the invention can be administered alone, or can be administered in combination with additional antibodies or chemotherapeutic drugs or radiation therapy. For example, a monoclonal, oligoclonal or polyclonal mixture of CD105 antibodies that block cell adhesion, invasion, angiogenesis or proliferation can be administered in combination with a drug shown to inhibit tumour cell proliferation. Moreover, the CD105 targeting agents of the invention can used in patients who have failed other chemotherapy treatments, for example, treatments that include anti-VEGF agents.

Another embodiment of the invention includes a method of diagnosing diseases or conditions in which an antibody as disclosed herein is utilised to detect the level of CD105 in a patient or patient sample. In one embodiment, the patient sample is blood or blood serum or urine. In further embodiments, methods for the identification of risk factors, diagnosis of disease, and staging of disease is presented which involves the identification of the expression and/or overexpression of CD105 using anti-CD105 antibodies. In some embodiments, the methods comprise administering to a patient a fully human antibody conjugate that selectively binds to CD105 on a cell. The antibody conjugate comprises an antibody that specifically binds to CD105 and a label. The methods further comprise observing the presence of the label in the patient. A relatively high amount of the label will indicate a relatively high risk of the disease and a relatively low amount of the label will indicate a relatively low risk of the disease. In one embodiment, the label is a green fluorescent protein.

The invention further provides methods for assaying the level of CD105 in a patient sample, comprising contacting an antibody as disclosed herein with a biological sample from a patient, and detecting the level of binding between said antibody and CD105 in said sample. In more specific embodiments, the biological sample is blood, plasma or serum.

Another embodiment of the invention includes a method for diagnosing a condition associated with the expression of CD105 in a cell by contacting the serum or a cell with an antibody as disclosed herein, and thereafter detecting the presence of CD105. In one embodiment the condition can be a proliferative, angiogenic, cell adhesion or invasion-related disease including, but not limited to, a neoplastic disease.

In another embodiment, the invention includes an assay kit for detecting CD105 in mammalian tissues, cells, or body fluids to screen for CD105-related diseases. The kit includes an antibody as disclosed herein and a means for indicating the reaction of the antibody with CD105, if present. In one embodiment the antibody is a monoclonal antibody. In one embodiment, the antibody that binds CD105 is labelled. In another embodiment the antibody is an unlabelled primary antibody and the kit further includes a means for detecting the primary antibody. In one embodiment, the means for detecting includes a labelled second antibody that is an anti-immunoglobulin. The antibody may be labelled with a marker selected from the group consisting of a fluorochrome, an enzyme, a radionuclide and a radiopaque material.

In some embodiments, the targeted binding agents or antibodies as disclosed herein can be modified to enhance their capability of fixing complement and participating in complement-dependent cytotoxicity (CDC). In other embodiments, the targeted binding agents or antibodies can be modified to enhance their capability of activating effector cells and participating in antibody-dependent cytotoxicity (ADCC). In yet other embodiments, the targeted binding agents or antibodies as disclosed herein can be modified both to enhance their capability of activating effector cells and participating in antibody-dependent cytotoxicity (ADCC) and to enhance their capability of fixing complement and participating in complement-dependent cytotoxicity (CDC).

In some embodiments, the targeted binding agents or antibodies as disclosed herein can be modified to reduce their capability of fixing complement and participating in complement-dependent cytotoxicity (CDC). In other embodiments, the targeted binding agents or antibodies can be modified to reduce their capability of activating effector cells and participating in antibody-dependent cytotoxicity (ADCC). In yet other embodiments, the targeted binding agents or antibodies as disclosed herein can be modified both to reduce their capability of activating effector cells and participating in antibody-dependent cytotoxicity (ADCC) and to reduce their capability of fixing complement and participating in complement-dependent cytotoxicity (CDC).

In certain embodiments, the half-life of a targeted binding agent or antibody as disclosed herein and of compositions of the invention is at least about 4 to 7 days. In certain embodiments, the mean half-life of a targeted binding agent or antibody as disclosed herein and of compositions of the invention is at least about 2 to 5 days, 3 to 6 days, 4 to 7 days, 5 to 8 days, 6 to 9 days, 7 to 10 days, 8 to 11 days, 8 to 12, 9 to 13, 10 to 14, 11 to 15, 12 to 16, 13 to 17, 14 to 18, 15 to 19, or 16 to 20 days. In other embodiments, the mean half-life of a targeted binding agent or antibody as disclosed herein and of compositions of the invention is at least about 17 to 21 days, 18 to 22 days, 19 to 23 days, 20 to 24 days, 21 to 25, days, 22 to 26 days, 23 to 27 days, 24 to 28 days, 25 to 29 days, or 26 to 30 days. In still further embodiments the half-life of a targeted binding agent or antibody as disclosed herein and of compositions of the invention can be up to about 50 days. In certain embodiments, the half-lives of antibodies and of compositions of the invention can be prolonged by methods known in the art. Such prolongation can in turn reduce the amount and/or frequency of dosing of the antibody compositions. Antibodies with improved in vivo half-lives and methods for preparing them are disclosed in U.S. Pat. No. 6,277,375; and International Publication Nos. WO 98/23289 and WO 97/3461.

In another embodiment, the invention provides an article of manufacture including a container. The container includes a composition containing a targeted binding agent or antibody as disclosed herein, and a package insert or label indicating that the composition can be used to treat cell adhesion, invasion, angiogenesis, and/or proliferation-related diseases, including, but not limited to, diseases characterised by the expression or overexpression of CD105.

In other embodiments, the invention provides a kit comprising a composition containing a targeted binding agent or antibody as disclosed herein, and instructions to administer the composition to a subject in need of treatment.

The present invention provides formulation of proteins comprising a variant Fc region. That is, a non-naturally occurring Fc region, for example an Fc region comprising one or more non naturally occurring amino acid residues. Also encompassed by the variant Fc regions of present invention are Fc regions which comprise amino acid deletions, additions and/or modifications.

The serum half-life of proteins comprising Fc regions may be increased by increasing the binding affinity of the Fc region for FcRn. In one embodiment, the Fc variant protein has enhanced serum half life relative to comparable molecule.

In another embodiment, the present invention provides an Fc variant, wherein the Fc region comprises at least one non naturally occurring amino acid at one or more positions selected from the group consisting of 239, 330 and 332, as numbered by the EU index as set forth in Kabat. In a specific embodiment, the present invention provides an Fc variant, wherein the Fc region comprises at least one non naturally occurring amino acid selected from the group consisting of 239D, 330L and 332E, as numbered by the EU index as set forth in Kabat. Optionally, the Fc region may further comprise additional non naturally occurring amino acid at one or more positions selected from the group consisting of 252, 254, and 256, as numbered by the EU index as set forth in Kabat. In a specific embodiment, the present invention provides an Fc variant, wherein the Fc region comprises at least one non naturally occurring amino acid selected from the group consisting of 239D, 330L and 332E, as numbered by the EU index as set forth in Kabat and at least one non naturally occurring amino acid at one or more positions selected from the group consisting of 252Y, 254T and 256E, as numbered by the EU index as set forth in Kabat.

In another embodiment, the present invention provides an Fc variant, wherein the Fc region comprises at least one non naturally occurring amino acid at one or more positions selected from the group consisting of 234, 235 and 331, as numbered by the EU index as set forth in Kabat. In a specific embodiment, the present invention provides an Fc variant, wherein the Fc region comprises at least one non naturally occurring amino acid selected from the group consisting of 234F, 235F, 235Y, and 331S, as numbered by the EU index as set forth in Kabat. In a further specific embodiment, an Fc variant of the invention comprises the 234F, 235F, and 331S non naturally occurring amino acid residues, as numbered by the EU index as set forth in Kabat. In another specific embodiment, an Fc variant of the invention comprises the 234F, 235Y, and 331S non naturally occurring amino acid residues, as numbered by the EU index as set forth in Kabat. Optionally, the Fc region may further comprise additional non naturally occurring amino acid at one or more positions selected from the group consisting of 252, 254, and 256, as numbered by the EU index as set forth in Kabat. In a specific embodiment, the present invention provides an Fc variant, wherein the Fc region comprises at least one non naturally occurring amino acid selected from the group consisting of 234F, 235F, 235Y, and 331S, as numbered by the EU index as set forth in Kabat; and at least one non naturally occurring amino acid at one or more positions are selected from the group consisting of 252Y, 254T and 256E, as numbered by the EU index as set forth in Kabat.

In another embodiment, the present invention provides an Fc variant protein formulation, wherein the Fc region comprises at least a non naturally occurring amino acid at one or more positions selected from the group consisting of 239, 330 and 332, as numbered by the EU index as set forth in Kabat. In a specific embodiment, the present invention provides an Fc variant protein formulation, wherein the Fc region comprises at least one non naturally occurring amino acid selected from the group consisting of 239D, 330L and 332E, as numbered by the EU index as set forth in Kabat. Optionally, the Fc region may further comprise additional non naturally occurring amino acid at one or more positions selected from the group consisting of 252, 254, and 256, as numbered by the EU index as set forth in Kabat. In a specific embodiment, the present invention provides an Fc variant protein formulation, wherein the Fc region comprises at least one non naturally occurring amino acid selected from the group consisting of 239D, 330L and 332E, as numbered by the EU index as set forth in Kabat and at least one non naturally occurring amino acid at one or more positions are selected from the group consisting of 252Y, 254T and 256E, as numbered by the EU index as set forth in Kabat.

In another embodiment, the present invention provides an Fc variant protein formulation, wherein the Fc region comprises at least one non naturally occurring amino acid at one or more positions selected from the group consisting of 234, 235 and 331, as numbered by the EU index as set forth in Kabat. In a specific embodiment, the present invention provides an Fc variant protein formulation, wherein the Fc region comprises at least one non naturally occurring amino acid selected from the group consisting of 234F, 235F, 235Y, and 331S, as numbered by the EU index as set forth in Kabat. Optionally, the Fc region may further comprise additional non naturally occurring amino acid at one or more positions selected from the group consisting of 252, 254, and 256, as numbered by the EU index as set forth in Kabat. In a specific embodiment, the present invention provides an Fc variant protein formulation, wherein the Fc region comprises at least one non naturally occurring amino acid selected from the group consisting of 234F, 235F, 235Y, and 331S, as numbered by the EU index as set forth in Kabat; and at least one non naturally occurring amino acid at one or more positions are selected from the group consisting of 252Y, 254T and 256E, as numbered by the EU index as set forth in Kabat.

Methods for generating non naturally occurring Fc regions are known in the art. For example, amino acid substitutions and/or deletions can be generated by mutagenesis methods, including, but not limited to, site-directed mutagenesis (Kunkel, Proc. Natl. Acad. Sci. USA 82:488-492 (1985)), PCR mutagenesis (Higuchi, in “PCR Protocols: A Guide to Methods and Applications”, Academic Press, San Diego, pp. 177-183 (1990)), and cassette mutagenesis (Wells et al., Gene 34:315-323 (1985)). Preferably, site-directed mutagenesis is performed by the overlap-extension PCR method (Higuchi, in “PCR Technology: Principles and Applications for DNA Amplification”, Stockton Press, New York, pp. 61-70 (1989)). The technique of overlap-extension PCR (Higuchi, ibid.) can also be used to introduce any desired mutation(s) into a target sequence (the starting DNA). For example, the first round of PCR in the overlap-extension method involves amplifying the target sequence with an outside primer (primer 1) and an internal mutagenesis primer (primer 3), and separately with a second outside primer (primer 4) and an internal primer (primer 2), yielding two PCR segments (segments A and B). The internal mutagenesis primer (primer 3) is designed to contain mismatches to the target sequence specifying the desired mutation(s). In the second round of PCR, the products of the first round of PCR (segments A and B) are amplified by PCR using the two outside primers (primers 1 and 4). The resulting full-length PCR segment (segment C) is digested with restriction enzymes and the resulting restriction fragment is cloned into an appropriate vector. As the first step of mutagenesis, the starting DNA (e.g., encoding an Fc fusion protein, an antibody or simply an Fc region), is operably cloned into a mutagenesis vector. The primers are designed to reflect the desired amino acid substitution. Other methods useful for the generation of variant Fc regions are known in the art (see, e.g., U.S. Pat. Nos. 5,624,821; 5,885,573; 5,677,425; 6,165,745; 6,277,375; 5,869,046; 6,121,022; 5,624,821; 5,648,260; 6,528,624; 6,194,551; 6,737,056; 6,821,505; 6,277,375; U.S. Patent Publication Nos. 2004/0002587 and PCT Publications WO 94/29351; WO 99/58572; WO 00/42072; WO 02/060919; WO 04/029207; WO 04/099249; WO 04/063351).

In some embodiments of the invention, the glycosylation patterns of the antibodies provided herein are modified to enhance ADCC and CDC effector function. See Shields R L et al., (2002) JBC. 277:26733; Shinkawa T et al., (2003) JBC. 278:3466 and Okazaki A et al., (2004) J. Mol. Biol., 336: 1239. In some embodiments, an Fc variant protein comprises one or more engineered glycoforms, i.e., a carbohydrate composition that is covalently attached to the molecule comprising an Fc region. Engineered glycoforms may be useful for a variety of purposes, including but not limited to enhancing or reducing effector function. Engineered glycoforms may be generated by any method known to one skilled in the art, for example by using engineered or variant expression strains, by co-expression with one or more enzymes, for example DI N-acetylglucosaminyltransferase III (GnTI11), by expressing a molecule comprising an Fc region in various organisms or cell lines from various organisms, or by modifying carbohydrate(s) after the molecule comprising Fc region has been expressed. Methods for generating engineered glycoforms are known in the art, and include but are not limited to those described in Umana et al, 1999, Nat. Biotechnol 17:176-180; Davies et al., 20017 Biotechnol Bioeng 74:288-294; Shields et al, 2002, J Biol Chem 277:26733-26740; Shinkawa et al., 2003, J Biol Chem 278:3466-3473) U.S. Pat. No. 6,602,684; U.S. Ser. No. 10/277,370; U.S. Ser. No. 10/113,929; PCT WO 00/61739A1; PCT WO 01/292246A1; PCT WO 02/311140A1; PCT WO 02/30954A1; Potillegent™ technology (Biowa, Inc. Princeton, N.J.); GlycoMAb™ glycosylation engineering technology (GLYCART biotechnology AG, Zurich, Switzerland). See, e.g., WO 00061739; EA01229125; US 20030115614; Okazaki et al., 2004, JMB, 336: 1239-49.

Accordingly, in one embodiment the Fc regions of anti-CD105 antibodies of the invention comprise altered glycosylation of amino acid residues. In another embodiment, the altered glycosylation of the amino acid residues results in lowered effector function. In another embodiment, the altered glycosylation of the amino acid residues results in increased effector function. In a specific embodiment, the Fc region has reduced fucosylation. In another embodiment, the Fc region is afucosylated (see for examples, U.S. Patent Application Publication No. 2005/0226867). In one aspect, these antibodies with increased effector function, specifically ADCC, as generated in host cells (e.g., CHO cells, Lemna minor) engineered to produce highly defucosylated antibody with over 100-fold higher ADCC compared to antibody produced by the parental cells (Mori et al., 2004, Biotechnol Bioeng 88:901-908; Cox et al., 2006, Nat Biotechnol., 24:1591-7).

It is also known in the art that the glycosylation of the Fc region can be modified to increase or decrease effector function (see for examples, Umana et al, 1999, Nat. Biotechnol 17:176-180; Davies et al., 2001, Biotechnol Bioeng 74:288-294; Shields et al, 2002, J Biol Chem 277:26733-26740; Shinkawa et al., 2003, J Biol Chem 278:3466-3473) U.S. Pat. No. 6,602,684; U.S. Ser. No. 10/277,370; U.S. Ser. No. 10/113,929; PCT WO 00/61739A1; PCT WO 01/292246A1; PCT WO 02/311140A1; PCT WO 02/30954A1; Potillegent™ technology (Biowa, Inc. Princeton, N.J.); GlycoMAb™ glycosylation engineering technology (GLYCART biotechnology AG, Zurich, Switzerland). Accordingly, in one embodiment the Fc regions of the antibodies of the invention comprise altered glycosylation of amino acid residues. In another embodiment, the altered glycosylation of the amino acid residues results in lowered effector function. In another embodiment, the altered glycosylation of the amino acid residues results in increased effector function. In a specific embodiment, the Fc region has reduced fucosylation. In another embodiment, the Fc region is afucosylated (see for examples, U.S. Patent Application Publication No. 2005/0226867).

### BRIEF DESCRIPTION OF THE DRAWINGS

FIG. 1 depicts a bar chart showing the results of a HUVEC proliferation assay for antibodies 4.37 and 4.120.

FIG. 2 depicts a bar chart showing the results of a HUVEC proliferation assay for antibodies 4D4, 6A6, 6B1, 6B10, 11H2, 9H10, 3C1 and 10C9.

FIG. 3 depicts a bar chart showing the effect of antibodies 4D4, 6B1, 6B10, and 10C9 on vessel length (mm) and number of bifurcations.

FIG. 4 depicts a bar chart showing the results of a binning study. Specifically the ability of antibodies 4D4, 6A6, 6B1, 6B10, 11H2, 9H10, 3C1, 4.37, 4.120, and 10C9 to block SN6 binding to HUVEC Cells.

FIG. 5 depicts a bar chart showing the results from a Colo205 matrigel plug assay measuring hemoglobin (hb) content for antibodies 4.120, 4D4, 6B10 and 4.37.

FIG. 6 depicts a bar chart showing the results from a Colo205 matrigel plug assay measuring positive CD31 staining for antibodies 4.120, 4D4, 6B10 and 4.37.

FIG. 7 depicts a bar chart showing the ADCC activity of antibodies 4D4, 6A6, 6B1, 6B10, 11H2, 9H10, 3C1, 4.37, 4.120, and 10C9.

FIG. 8 depicts a bar chart showing the CDC activity for antibody 4.120.

FIG. 9 depicts a bar chart showing internalization results for antibodies 4D4, 6A6, 6B1, 6B10, 11H2, 9H10, 3C1, 4.37, 4.120, and 10C9.

### DETAILED DESCRIPTION OF THE PREFERRED EMBODIMENT

Embodiments of the invention relate to a novel set of CD105 blocking molecules, such as, for example, antibodies, that inhibit TGF-beta signaling. Such molecules can be used as single agents, or alternatively, in combination with other binding antibodies/agents. They can also be used in combination with any standard or novel anti-cancer agents.

Embodiments of the invention relate to targeted binding agents that bind to CD105. In some embodiments, the targeted binding agents bind to CD105 and inhibit the binding of a CD105 ligand such as TGF-13 to its receptor, CD105. In some embodiments, this binding can neutralize, block, inhibit, abrogate, or interfere with one or more aspects of CD105-associated effects. In one embodiment, the targeted binding agents are monoclonal antibodies, or binding fragments thereof. Such monoclonal antibodies may be referred to as anti-CD105 antibodies herein.

Other embodiments of the invention include fully human anti-CD105 antibodies, and antibody preparations that are therapeutically useful. In one embodiment, preparations of the anti-CD105 antibody of the invention have desirable therapeutic properties, including strong binding affinity for CD105, the ability to promote endothelial cell apoptosis or inhibit proliferation of endothelial cells, modulate cytoskeletal organization, inhibit tube formation and the ability to induce endothelial cell cytotoxicity via ADCC and/or CDC activity.

In addition, embodiments of the invention include methods of using these antibodies for treating diseases. Anti-CD105 antibodies of the invention are useful for preventing CD105-mediated tumourigenesis and tumour invasion of healthy tissue. In addition CD105 antibodies can be useful for treating diseases associated with angiogenesis such as ocular disease such as AMD, inflammatory disorders such as rheumatoid arthritis, and cardiovascular disease and sepsis as well as neoplastic diseases. Any disease that is characterized by any type of malignant tumour, including metastatic cancers, lymphatic tumours, and blood cancers, can also be treated by this inhibition mechanism. Exemplary cancers in humans include a bladder tumour, renal cell cancer, breast tumour, prostate tumour, basal cell carcinoma, biliary tract cancer, bladder cancer, bone cancer, brain and CNS cancer (e.g., glioma tumour), cervical cancer, choriocarcinoma, colon and rectum cancer, connective tissue cancer, cancer of the digestive system; endometrial cancer, esophageal cancer; eye cancer; cancer of the head and neck; gastric cancer; intra-epithelial neoplasm; kidney cancer; larynx cancer; leukemia; liver cancer; lung cancer (e.g. small cell and non-small cell); lymphoma including Hodgkin's and Non-Hodgkin's lymphoma; melanoma; myeloma, neuroblastoma, oral cavity cancer (e.g., lip, tongue, mouth, and pharynx); ovarian cancer; pancreatic cancer, retinoblastoma; rhabdomyosarcoma; rectal cancer, renal cancer, cancer of the respiratory system; sarcoma, skin cancer; stomach cancer, testicular cancer, thyroid cancer; uterine cancer, cancer of the urinary system, as well as other carcinomas and sarcomas. Malignant disorders commonly diagnosed in dogs, cats, and other pets include, but are not limited to, lymphosarcoma, osteosarcoma, mammary tumours, mastocytoma, brain tumour, melanoma, adenosquamous carcinoma, carcinoid lung tumour, bronchial gland tumour, bronchiolar adenocarcinoma, fibroma, myxochondroma, pulmonary sarcoma, neurosarcoma, osteoma, papilloma, retinoblastoma, Ewing's sarcoma, Wilm's tumour, Burkitt's lymphoma, microglioma, neuroblastoma, osteoclastoma, oral neoplasia, fibrosarcoma, osteosarcoma and rhabdomyosarcoma, genital squamous cell carcinoma, transmissible venereal tumour, testicular tumour, seminoma, Sertoli cell tumour, hemangiopericytoma, histiocytoma, chloroma (e.g., granulocytic sarcoma), corneal papilloma, corneal squamous cell carcinoma, hemangiosarcoma, pleural mesothelioma, basal cell tumour, thymoma, stomach tumour, adrenal gland carcinoma, oral papillomatosis, hemangioendothelioma and cystadenoma, follicular lymphoma, intestinal lymphosarcoma, fibrosarcoma and pulmonary squamous cell carcinoma. In rodents, such as a ferret, exemplary cancers include insulinoma, lymphoma, sarcoma, neuroma, pancreatic islet cell tumour, gastric MALT lymphoma and gastric adenocarcinoma. Neoplasias affecting agricultural livestock include leukemia, hemangiopericytoma and bovine ocular neoplasia (in cattle); preputial fibrosarcoma, ulcerative squamous cell carcinoma, preputial carcinoma, connective tissue neoplasia and mastocytoma (in horses); hepatocellular carcinoma (in swine); lymphoma and pulmonary adenomatosis (in sheep); pulmonary sarcoma, lymphoma, Rous sarcoma, reticulo-endotheliosis, fibrosarcoma, nephroblastoma, B-cell lymphoma and lymphoid leukosis (in avian species); retinoblastoma, hepatic neoplasia, lymphosarcoma (lymphoblastic lymphoma), plasmacytoid leukemia and swimbladder sarcoma (in fish), caseous lumphadenitis (CLA): chronic, infectious, contagious disease of sheep and goats caused by the bacterium Corynebacterium pseudotuberculosis, and contagious lung tumour of sheep caused by jaagsiekte.

Other embodiments of the invention include diagnostic assays for specifically determining the quantity of CD105 in a biological sample. The assay kit can include a targeted binding agent or antibody as disclosed herein along with the necessary labels for detecting such antibodies. These diagnostic assays are useful to screen for cell adhesion, invasion, angiogenesis or proliferation-related diseases including, but not limited to, neoplastic diseases. Another aspect of the invention is an antagonist of the biological activity of CD105 wherein the antagonist binds to CD105. In one embodiment, the antagonist is a targeted binding agent, such as an antibody. The antagonist may be selected from an antibody described herein, for example, antibody 4.120, 9H10, 10C9, 4D4, 11H2, 6B1, 4.37, 6B10, 3C1, and 6A6.

In one embodiment the antagonist of the biological activity of CD105 may bind to CD105 and thereby inhibit or suppress ligand binding to the CD105 receptor, thereby inhibiting tumor angiogenesis and/or cellular proliferation.

One embodiment is a targeted binding agent which binds to the same epitope or epitopes as fully human monoclonal antibody 4.120, 9H10, 10C9, 4D4, 11H2, 6B1, 4.37, 6B10, 3C1, and 6A6.

One embodiment is an antibody which binds to the same epitope or epitopes as fully human monoclonal antibody 4.120, 9H10, 10C9, 4D4, 11H2, 6B1, 4.37, 6B10, 3C1, and 6A6.

One embodiment is a hybridoma that produces the targeted binding agent as described hereinabove. In one embodiment is a hybridoma that produces the light chain and/or the heavy chain of the antibodies as described hereinabove. In one embodiment the hybridoma produces the light chain and/or the heavy chain of a fully human monoclonal antibody. In another embodiment the hybridoma produces the light chain and/or the heavy chain of fully human monoclonal antibody 4.120, 9H10, 10C9, 4D4, 11H2, 6B1, 4.37, 6B10, 3C1, and 6A6. Alternatively the hybridoma may produce an antibody which binds to the same epitope or epitopes as fully human monoclonal antibody 4.120, 9H10, 10C9, 4D4, 11H2, 6B1, 4.37, 6B10, 3C1, and 6A6.

Another embodiment is a nucleic acid molecule encoding the targeted binding agent as described hereinabove. In one embodiment is a nucleic acid molecule encoding the light chain or the heavy chain of an antibody as described hereinabove. In one embodiment the nucleic acid molecule encodes the light chain or the heavy chain of a fully human monoclonal antibody. Still another embodiment is a nucleic acid molecule encoding the light chain or the heavy chain of a fully human monoclonal antibody selected from antibodies 4.120, 9H10, 10C9, 4D4, 11H2, 6B1, 4.37, 6B10, 3C1, and 6A6.

Another embodiment of the invention is a vector comprising a nucleic acid molecule or molecules as described hereinabove, wherein the vector encodes a targeted binding agent as defined hereinabove. In one embodiment of the invention is a vector comprising a nucleic acid molecule or molecules as described hereinabove, wherein the vector encodes a light chain and/or a heavy chain of an antibody as defined hereinabove.

Yet another embodiment of the invention is a host cell comprising a vector as described hereinabove. Alternatively the host cell may comprise more than one vector.

In addition, one embodiment of the invention is a method of producing a targeted binding agent of the invention by culturing host cells under conditions wherein a nucleic acid molecule is expressed to produce the targeted binding agent, followed by recovery of the targeted binding agent. In one embodiment of the invention is a method of producing an antibody of the invention by culturing host cells under conditions wherein a nucleic acid molecule is expressed to produce the antibody, followed by recovery of the antibody.

In one embodiment the invention includes a method of making an targeted binding agent by transfecting at least one host cell with at least one nucleic acid molecule encoding the targeted binding agent as described hereinabove, expressing the nucleic acid molecule in the host cell and isolating the targeted binding agent. In one embodiment the invention includes a method of making an antibody by transfecting at least one host cell with at least one nucleic acid molecule encoding the antibody as described hereinabove, expressing the nucleic acid molecule in the host cell and isolating the antibody.

According to another aspect, the invention includes a method of antagonising the biological activity of CD105 by administering an antagonist as described herein. The method may include selecting an animal in need of treatment for angiogenesis and/or proliferation, and administering to the animal a therapeutically effective dose of an antagonist of the biological activity of CD105.

Another aspect of the invention includes a method of antagonising the biological activity of CD105 by administering a targeted binding agent as described hereinabove. The method may include selecting an animal in need of treatment for angiogenesis and/or proliferation, and administering to the animal a therapeutically effective dose of a targeted binding agent which antagonises the biological activity of CD105.

Another aspect of the invention includes a method of antagonising the biological activity of CD105 by administering an antibody as described hereinabove. The method may include selecting an animal in need of treatment for angiogenesis and/or proliferation, and administering to the animal a therapeutically effective dose of an antibody which antagonises the biological activity of CD105.

According to another aspect there is provided a method of treating angiogenesis and/or proliferation in an animal by administering a therapeutically effective amount of an antagonist of the biological activity of CD105. The method may include selecting an animal in need of treatment for angiogenesis and/or proliferation, and administering to the animal a therapeutically effective dose of an antagonist of the biological activity of CD105.

According to another aspect there is provided a method of treating angiogenesis and/or proliferation in an animal by administering a therapeutically effective amount of a targeted binding agent which antagonizes the biological activity of CD105. The method may include selecting an animal in need of treatment for angiogenesis and/or proliferation, and administering to the animal a therapeutically effective dose of a targeted binding agent which antagonises the biological activity of CD105. The targeted binding agent can be administered alone, or can be administered in combination with additional antibodies or chemotherapeutic drugs or radiation therapy.

According to another aspect there is provided a method of treating angiogenesis and/or proliferation in an animal by administering a therapeutically effective amount of an antibody which antagonizes the biological activity of CD105. The method may include selecting an animal in need of treatment for angiogenesis and/or proliferation, and administering to the animal a therapeutically effective dose of an antibody which antagonises the biological activity of CD105. The antibody can be administered alone, or can be administered in combination with additional antibodies or chemotherapeutic drugs or radiation therapy.

According to another aspect there is provided a method of treating cancer in an animal by administering a therapeutically effective amount of an antagonist of the biological activity of CD105. The method may include selecting an animal in need of treatment for cancer, and administering to the animal a therapeutically effective dose of an antagonist which antagonises the biological activity of CD105. The antagonist can be administered alone, or can be administered in combination with additional antibodies or chemotherapeutic drugs or radiation therapy.

According to another aspect there is provided a method of treating cancer in an animal by administering a therapeutically effective amount of a targeted binding agent which antagonizes the biological activity of CD105. The method may include selecting an animal in need of treatment for cancer, and administering to the animal a therapeutically effective dose of a targeted binding agent which antagonises the biological activity of CD105. The targeted binding agent can be administered alone, or can be administered in combination with additional antibodies or chemotherapeutic drugs or radiation therapy.

According to another aspect there is provided a method of treating cancer in an animal by administering a therapeutically effective amount of an antibody which antagonizes the biological activity of CD105. The method may include selecting an animal in need of treatment for cancer, and administering to the animal a therapeutically effective dose of an antibody which antagonises the biological activity of CD105. The antibody can be administered alone, or can be administered in combination with additional antibodies or chemotherapeutic drugs or radiation therapy.

According to another aspect there is provided a method of reducing or inhibiting tumour cell proliferation, adhesion, invasion and/or angiogenesis, in an animal by administering a therapeutically effective amount of an antibody which antagonizes the biological activity of CD105. The method may include selecting an animal in need of a reduction or inhibition of proliferation, cell adhesion, invasion and/or angiogenesis, and administering to the animal a therapeutically effective dose of an antibody which antagonises the biological activity of CD105. The antibody can be administered alone, or can be administered in combination with additional antibodies or chemotherapeutic drugs or radiation therapy.

According to another aspect there is provided a method of reducing tumour growth and/or metastasis, in an animal by administering a therapeutically effective amount of an antibody which antagonizes the biological activity of CD105. The method may include selecting an animal in need of a reduction of tumour growth and/or metastasis, and administering to the animal a therapeutically effective dose of an antibody which antagonises the biological activity of CD105. The antibody can be administered alone, or can be administered in combination with additional antibodies or chemotherapeutic drugs or radiation therapy.

According to another aspect of the invention there is provided the use of an antagonist of the biological activity of CD105 for the manufacture of a medicament for the treatment of tumor angiogenesis and/or cellular proliferation. In one embodiment the antagonist of the biological activity of CD105 is a targeted binding agent of the invention. In one embodiment the antagonist of the biological activity of CD105 is an antibody of the invention.

According to another aspect of the invention there is provided an antagonist of the biological activity of CD105 for use as a medicament for the treatment of tumor angiogenesis and/or cellular proliferation. In one embodiment the antagonist of the biological activity of CD105 is a targeted binding agent of the invention. In one embodiment the antagonist of the biological activity of CD105 is an antibody of the invention.

According to another aspect of the invention there is provided the use of a targeted binding agent or an antibody which antagonizes the biological activity of CD105 for the manufacture of a medicament for the treatment of angiogenesis and/or proliferation.

According to another aspect of the invention there is provided a targeted binding agent or an antibody which antagonizes the biological activity of CD105 for use as a medicament for the treatment of angiogenesis and/or proliferation.

According to another aspect of the invention there is provided the use of a targeted binding agent or an antibody which antagonizes the biological activity of CD105 for the manufacture of a medicament for the treatment of disease-related angiogenesis and/or proliferation.

According to another aspect of the invention there is provided an antibody which antagonizes the biological activity of CD105 for use as a medicament for the treatment of disease-related angiogenesis and/or proliferation.

According to another aspect of the invention there is provided the use of an antagonist of the biological activity of CD105 for the manufacture of a medicament for the treatment of cancer in a mammal. In one embodiment the antagonist of the biological activity of CD105 is a targeted binding agent of the invention. In one embodiment the antagonist of the biological activity of CD105 is an antibody of the invention.

According to another aspect of the invention there is provided an antagonist of the biological activity of CD105 for use as a medicament for the treatment of cancer in a mammal. In one embodiment the antagonist of the biological activity of CD105 is a targeted binding agent of the invention. In one embodiment the antagonist of the biological activity of CD105 is an antibody of the invention.

According to another aspect of the invention there is provided the use of a targeted binding agent which antagonizes the biological activity of CD105 for the manufacture of a medicament for the treatment of cancer in a mammal.

According to another aspect of the invention there is provided a targeted binding agent which antagonizes the biological activity of CD105 for use as a medicament for the treatment of cancer in a mammal.

According to another aspect of the invention there is provided the use of an antibody which antagonizes the biological activity of CD105 for the manufacture of a medicament for the treatment of cancer in a mammal.

According to another aspect of the invention there is provided an antibody which antagonizes the biological activity of CD105 for use as a medicament for the treatment of cancer in a mammal.

According to another aspect there is provided the use of a targeted binding agent or an antibody which antagonizes the biological activity of CD105 for the manufacture of a medicament for the reduction or inhibition proliferation, and/or angiogenesis in an animal.

According to another aspect there is provided a targeted binding agent or an antibody which antagonizes the biological activity of CD105 for use as a medicament for the reduction or inhibition proliferation, and/or angiogenesis in an animal.

According to another aspect there is provided the use of a targeted binding agent or an antibody which antagonizes the biological activity of CD105 for the manufacture of a medicament for reducing tumour growth and/or metastasis, in an animal.

According to another aspect there is provided a targeted binding agent or an antibody which antagonizes the biological activity of CD105 for use as a medicament for reducing tumour growth and/or metastasis, in an animal.

In one embodiment the present invention is particularly suitable for use in antagonizing CD105, in patients with a tumour which is dependent alone, or in part, on CD105 receptor signalling.

According to another aspect of the invention there is provided a pharmaceutical composition comprising an antagonist of the biological activity of CD105, and a pharmaceutically acceptable carrier. In one embodiment the antagonist comprises an antibody. According to another aspect of the invention there is provided a pharmaceutical composition comprising an antagonist of the biological activity of CD105, and a pharmaceutically acceptable carrier. In one embodiment the antagonist comprises an antibody.

In some embodiments, following administration of the antibody that specifically binds to CD105, a clearing agent is administered, to remove excess circulating antibody from the blood.

Anti-CD105 antibodies are useful in the detection of CD105 in patient samples and accordingly are useful as diagnostics for disease states as described herein. In addition, based on their ability to significantly inhibit CD105-mediated signaling activity (as demonstrated in the Examples below), anti-CD105 antibodies have therapeutic effects in treating symptoms and conditions resulting from CD105 expression. In specific embodiments, the antibodies and methods herein relate to the treatment of symptoms resulting from CD105 induced angiogenesis, proliferation and/or intracellular signaling. Further embodiments involve using the antibodies and methods described herein to treat angiogenesis and/or proliferation-related diseases including neoplastic diseases, such as, melanoma, small cell lung cancer, non-small cell lung cancer, glioma, hepatocellular (liver) carcinoma, thyroid tumour, gastric (stomach) cancer, prostate cancer, breast cancer, ovarian cancer, bladder cancer, lung cancer, glioblastoma, endometrial cancer, kidney cancer, colon cancer, and pancreatic cancer. The antibodies may also be useful in treating cell adhesion and/or invasion in arthritis, atherosclerosis and diseases involving angiogenesis.

Another embodiment of the invention includes an assay kit for detecting CD105 in mammalian tissues, cells, or body fluids to screen for cell adhesion-, invasion-, angiogenesis- or proliferation related diseases. The kit includes a targeted binding agent that binds to CD105 and a means for indicating the reaction of the targeted binding agent with CD105, if present. In one embodiment, the targeted binding agent that binds CD105 is labeled. In another embodiment the targeted binding agent is an unlabeled and the kit further includes a means for detecting the targeted binding agent. Preferably the targeted binding agent is labeled with a marker selected from the group consisting of a fluorochrome, an enzyme, a radionuclide and a radio-opaque material.

Another embodiment of the invention includes an assay kit for detecting CD105 in mammalian tissues, cells, or body fluids to screen for cell adhesion-, invasion-, angiogenesis or proliferation-related diseases. The kit includes an antibody that binds to CD105 and a means for indicating the reaction of the antibody with CD105, if present. The antibody may be a monoclonal antibody. In one embodiment, the antibody that binds CD105 is labeled. In another embodiment the antibody is an unlabeled primary antibody and the kit further includes a means for detecting the primary antibody. In one embodiment, the means includes a labeled second antibody that is an anti-immunoglobulin. Preferably the antibody is labeled with a marker selected from the group consisting of a fluorochrome, an enzyme, a radionuclide and a radio-opaque material.

Further embodiments, features, and the like regarding the antibodies as disclosed herein are provided in additional detail below.

### Sequence Listing

Embodiments of the invention include the specific antibodies listed below in Table 1. This table reports the identification number of each anti-CD105 antibody, along with the SEQ ID number of the variable domain of the corresponding heavy chain and light chain genes and polypeptides, respectively. Each antibody has been given an identification number.

TABLE 1

| mAb | | SEQ ID |
|---|---|---|
| ID No.: | Sequence | NO: |
| 4.120 | Nucleotide sequence encoding the variable region of the heavy chain | 1 |
| | Amino acid sequence encoding the variable region of the heavy chain | 2 |
| | Nucleotide sequence encoding the variable region of the light chain | 3 |
| | Amino acid sequence encoding the variable region of the light chain | 4 |
| 9H10 | Nucleotide sequence encoding the variable region of the heavy chain | 5 |
| | Amino acid sequence encoding the variable region of the heavy chain | 6 |
| | Nucleotide sequence encoding the variable region of the light chain | 7 |
| | Amino acid sequence encoding the variable region of the light chain | 8 |
| 10C9 | Nucleotide sequence encoding the variable region of the heavy chain | 9 |
| | Amino acid sequence encoding the variable region of the heavy chain | 10 |
| | Nucleotide sequence encoding the variable region of the light chain | 11 |
| | Amino acid sequence encoding the variable region of the light chain | 12 |
| 4D4 | Nucleotide sequence encoding the variable region of the heavy chain | 13 |
| | Amino acid sequence encoding the variable region of the heavy chain | 14 |
| | Nucleotide sequence encoding the variable region of the light chain | 15 |
| | Amino acid sequence encoding the variable region of the light chain | 16 |
| 11H2 | Nucleotide sequence encoding the variable region of the heavy chain | 17 |
| | Amino acid sequence encoding the variable region of the heavy chain | 18 |
| | Nucleotide sequence encoding the variable region of the light chain | 19 |
| | Amino acid sequence encoding the variable region of the light chain | 20 |
| 6B1 | Nucleotide sequence encoding the variable region of the heavy chain | 21 |
| | Amino acid sequence encoding the variable region of the heavy chain | 22 |
| | Nucleotide sequence encoding the variable region of the light chain | 23 |
| | Amino acid sequence encoding the variable region of the light chain | 24 |
| 4.37 | Nucleotide sequence encoding the variable region of the heavy chain | 25 |
| | Amino acid sequence encoding the variable region of the heavy chain | 26 |
| | Nucleotide sequence encoding the variable region of the light chain | 27 |
| | Amino acid sequence encoding the variable region of the light chain | 28 |
| 6B10 | Nucleotide sequence encoding the variable region of the heavy chain | 29 |
| | Amino acid sequence encoding the variable region of the heavy chain | 30 |
| | Nucleotide sequence encoding the variable region of the light chain | 31 |
| | Amino acid sequence encoding the variable region of the light chain | 32 |
| 3C1 | Nucleotide sequence encoding the variable region of the heavy chain | 33 |
| | Amino acid sequence encoding the variable region of the heavy chain | 34 |
| | Nucleotide sequence encoding the variable region of the light chain | 35 |
| | Amino acid sequence encoding the variable region of the light chain | 36 |
| 6A6 | Nucleotide sequence encoding the variable region of the heavy chain | 37 |
| | Amino acid sequence encoding the variable region of the heavy chain | 38 |
| | Nucleotide sequence encoding the variable region of the light chain | 39 |
| | Amino acid sequence encoding the variable region of the light chain | 40 |

Table 2 is a table comparing the antibody heavy chain regions to their cognate germline heavy chain region and the antibody kappa light chain regions to their cognate germ line light chain region.

TABLE 2

| | | | | | | | | | | | | |
|---|---|---|---|---|---|---|---|---|---|---|---|---|
| SEQ ID No | Chain | Chain | V | D | J | FR1 | CDR1 | FR2 | CDR2 | FR3 | CDR3 | FR4 |

| | | | | | | | | | | | | |
|---|---|---|---|---|---|---|---|---|---|---|---|---|
| 2 | 4.120VH | 4.120VH | | | | QVQLQESGPGLVKPS | SYYWS | WIRQPAGK | RIYTSGSTNY | RVTMSVDTSKNQFSLKLS | RDIGATIKGFDY | WGQGTLVTVSS |
| | | | | | | ETLSLTCTVSGGSIS | | GLEWIG | NPSLKS | SVTAADTAVYYCAR | | |
| 41 | Germline | Germline | VH4-59 | D5-12 | JH4 | QVQLQESGPGLVKPS | SYYWS | WIRQPPGK | YIYYSGSTNY | RVTISVDTSKNQFSLKLS | -DIVATI- | WGQGTLVTVSS |
| | | | | | | ETLSLTCTVSGGSIS | | GLEWIG | NPSLKS | SVTAADTAVYYCAR | YFDY | |
| 4 | 4.120Vk | 4.120Vk | | | | DIQMTQSPSSLSASV | RASQSISSY | WYQQKPGK | AASSLQS | GVPSRFSGSGSGTDFTLT | QQSYSTP-T | FGQGTRLEIK |
| | | | | | | GDRVTITC | LN | APKLLIY | | ISSLQPEDFATYYC | | |
| 42 | Germline | Germline | VkO2/O12 | | Jk5 | DIQMTQSPSSLSASV | RASQSISSY | WYQQKPGK | AASSLQS | GVPSRFSGSGSGTDFTLT | QQSYSTPIT | FGQGTRLEIK |
| | | | | | | GDRVTITC | LN | APKLLIY | | ISSLQPEDFATYYC | | |
| 6 | 9H10VH | 9H10VH | | | | QVQLVESGGGVVQPG | NYGMH | WVRQAPGK | VISYDGSNKY | RFTISRDNSKNTLYLQMN | DLMGATLFDN | WGQGTLVTVSS |
| | | | | | | RSLRLSCAASGFAFI | | GLDWVA | YTDSVKG | SLRAEDTAVYYCAR | | |
| 43 | Germline | Germline | VH3-30*01 | D1-26 | JH4 | QVQLVESGGGVVQPG | SYGMH | WVRQAPGK | VISYDGSNKY | RFTISRDNSKNTLYLQMN | ---GATYFDY | WGQGTLVTVSS |
| | | | | | | RSLRLSCAASGFTFS | | GLEWVA | YADSVKG | SLRAEDTAVYYCAR | | |
| 8 | 9H10VL | 9H10VL | | | | SYVLTQPPSVSVAPG | GGNNIGSKS | WFQQKPGQ | DDSDRPS | GIPERFSGSNSGNTATLT | QVWDSSSDHVV | FGGGTKLTVL |
| | | | | | | QTARISC | VH | APVLVVY | | ISRVEAGDEADYYC | | |
| 44 | Germline | Germline | V2-14 | | JL2 | SYVLTQPPSVSVAPG | GGNNIGSKS | WYQQKPGQ | DDSDRPS | GIPERFSGSNSGNTATLT | QVWDSSSDHVV | FGGGTKLTVL |
| | | | | | | QTARITC | VH | APVLVVY | | ISRVEAGDEADYYC | | |
| 10 | 10C9VH | 10C9VH | | | | QVQLVESGGGVVQPG | SYGMH | WVRQAPGK | VIWYDGSNKY | RFTISRDNSKNTLDLQMN | DRIAAARYYNG | WGQGTTVTVSS |
| | | | | | | RSLRLSCAASGFTFR | | GLEWVA | YADSVKG | SLRAEDTAVYYCAR | MDV | |
| 45 | Germline | Germline | VH3-33 | D6-13 | JH6 | QVQLVESGGGVVQPG | SYGMH | WVRQAPGK | VIWYDGSNKY | RFTISRDNSKNTLYLQMN | --IAAA- | WGQGTTVTVSS |
| | | | | | | RSLRLSCAASGFTFS | | GLEWVA | YADSVKG | SLRAEDTAVYYCAR | YYYGMDV | |
| 12 | 10C9Vk | 10C9Vk | | | | AIVMTQSPDSLAVSL | KSSQSVLYS | WYQQKPGQ | WASTRES | GVPDRFSVSGSGTDFTLT | QQYYDSPLT | FGGGTKVEIK |
| | | | | | | GERATINC | SNNKNYLA | PPNLLFY | | ISSLQAEDVAVYYC | | |
| 46 | Germline | Germline | VkB3 | | Jk4 | DIVMTQSPDSLAVSL | KSSQSVLYS | WYQQKPGQ | WASTRES | GVPDRFSGSGSGTDFTLT | QQYYSTPLT | FGGGTKVEIK |
| | | | | | | GERATINC | SNNKNYLA | PPKLLIY | | ISSLQAEDVAVYYC | | |
| 14 | 4D4VH | 4D4VH | | | | QVQLVESGGGVVQPG | SYGMH | WVRQAPGK | VIWYDGSNKY | RFTISRDNSKNTLYLQMN | VLGATGGYYYY | WGQGTTVTVSS |
| | | | | | | RSLRLSCAASGFTFS | | GLEWVA | YADSVKG | SLRAEDTAVYYCAR | YGMDV | |
| 47 | Germline | Germline | VH3-33 | D1-26 | JH6 | QVQLVESGGGVVQPG | SYGMH | WVRQAPGK | VIWYDGSNKY | RFTISRDNSKNTLYLQMN | --GAT-- | WGQGTTVTVSS |
| | | | | | | RSLRLSCAASGFTFS | | GLEWVA | YADSVKG | SLRAEDTAVYYCAR | YYYYYGMDV | |
| 16 | 4D4VL | 4D4VL | | | | SYELTQPPSVSVSPG | SGDKLGDKY | WYQQKPGQ | QDIKRPS | GIPERFSGSKSGNTATLT | QAWDSST-VV | FGGGTKLTVL |
| | | | | | | QTASITC | AC | SPVLVIY | | ISGTQAMDEADYYC | | |
| 48 | Germline | Germline | V2-1 | | JL2 | SYELTQPPSVSVSPG | SGDKLGDKY | WYQQKPGQ | QDSKRPS | GIPERFSGSNSGNTATLT | QAWDSSTAVV | FGGGTKLTVL |
| | | | | | | QTASITC | AC | SPVLVIY | | ISGTQAMDEADYYC | | |
| 18 | 11H2VH | 11H2VH | | | | QVQLVESGGGVVQPG | SYGMH | WVRQAPGK | IIWYDGSYKY | RFTISRDNSKNTLSLQMN | DGKYPFDY | WGQGTLVTVSS |
| | | | | | | RSLRLSCAASGFSFS | | GLDWVA | YADSVKG | SLRAEDTAVYYCAR | | |
| 49 | Germline | Germline | VH3-33 | D5-24 | JH4 | QVQLVESGGGVVQPG | SYGMH | WVRQAPGK | VIWYDGSNKY | RFTISRDNSKNTLYLQMN | DG--YFDY | WGQGTLVTVSS |
| | | | | | | RSLRLSCAASGFTFS | | GLDWVA | YADSVKG | SLRAEDTAVYYCAR | | |
| 20 | 11H2VL | 11H2VL | | | | SYVLTQPPSVSVAPG | GGNNIGSKS | WYQQKPGQ | DDSDRPS | GIPERFSGSNSGNTATLT | QVWDRSSDHVV | FGGGTKLTVL |
| | | | | | | QTARITC | VH | APVLVVY | | ISRVEAGDEADYYC | | |
| 50 | Germline | Germline | V2-14 | | JL2 | SYVLTQPPSVSVAPG | GGNNIGSKS | WYQQKPGQ | DDSDRPS | GIPERFSGSNSGNTATLT | QVWDSSSDHVV | FGGGTKLTVL |
| | | | | | | QTARITC | VH | APVLVVY | | ISRVEAGDEADYYC | | |
| 22 | 6B1VH | 6B1VH | | | | QVQLVESGGGVVQPG | SYGMH | WVRQAPGK | VIWYDGSNKY | RFTISRDNSKNTLYLQMN | DYSSGWY | WGQGTLVTVSS |
| | | | | | | RSLRLSCAASGFTFS | | GLEWVA | YADSVKG | SLRAEDTAVYYCVR | | |
| 51 | Germline | Germline | VH3-33 | D6-19 | JH4 | QVQLVESGGGVVQPG | SYGMH | WVRQAPGK | VIWYDGSNKY | RFTISRDNSKNTLYLQMN | -YSSGWY | WGQGTLVTVSS |
| | | | | | | RSLRLSCAASGFTFS | | GLEWVA | YADSVKG | SLRAEDTAVYYCAR | | |
| 24 | 6B1VL | 6B1VL | | | | SYELTQPPSVSVSPG | SGDALPKKY | WYQQKSGQ | EDSKRPS | GIPERFSGSSSGTMATLT | YSIDSSVNHVV | FGGGTKLTVL |
| | | | | | | QTARITC | AY | APVLVIY | | ISGAQVEDEADYSC | | |
| 52 | Germline | Germline | V2-7 | | JL2 | SYELTQPPSVSVSPG | SGDALPKKY | WYQQKSGQ | EDSKRPS | GIPERFSGSSSGTMATLT | YSTDSSGNHVV | FGGGTKLTVL |
| | | | | | | QTARITC | AY | APVLVIY | | ISGAQVEDEADYYC | | |
| 26 | 4.37VH | 4.37VH | | | | QVQLVESGGGVVQPG | DYGMH | WVRQAPGK | VIWYDGSNKY | RFTISRDNSKNTLYLQMN | AAGFYYYYGMDV | WGQGTTVTVSS |
| | | | | | | RSLRLSCAASGFTFS | | GLEWVA | YADSVKG | SLRAEDTAVYYCAR | | |
| 53 | Germline | Germline | VH3-33 | D6-13 | JH6 | QVQLVESGGGVVQPG | SYGMH | WVRQAPGK | VIWYDGSNKY | RFTISRDNSKNTLYLQMN | AAGYYYYYGMDV | WGQGTTVTVSS |
| | | | | | | RSLRLSCAASGFTFS | | GLEWVA | YADSVKG | SLRAEDTAVYYCAR | | |
| 28 | 4.37Vk | 4.37Vk | | | | DIVMTQSPLSLPVTP | RSSQSLLYS | WYLQKPGQ | LGSNRAS | GVPDRFSGSGSGTDFTLK | MRALQTPFT | FGPGTKVDIK |
| | | | | | | GEPASISC | NGYNYLD | SPQLLIY | | ISRVEAEDVGVYYC | | |
| 54 | Germline | Germline | VkA3/A19 | | Jk3 | DIVMTQSPLSLPVTP | RSSQSLLHS | WYLQKPGQ | LGSNRAS | GVPDRFSGSGSGTDFTLK | MQALQTPFT | FGPGTKVDIK |
| | | | | | | GEPASISC | NGYNYLD | SPQLLIY | | ISRVEAEDVGVYYC | | |
| 30 | 6B10.1VH | 6B10.1VH | | | | QEQLVESGGGVVQPG | NYGIH | WVRQAPGK | VISYDGSKKY | RFTISRDNSKNTLYLQMN | AFSTMVRGVDH | WGQGTLVTVSS |
| | | | | | | RSLRLSCTASGFTFS | | GLEWVT | YADSVKG | SLRAEDTAVYYCAR | | |
| 55 | Germline | Germline | VH3-30*01 | D3-10 | JH4 | QVQLVESGGGVVQPG | SYGMH | WVRQAPGK | VISYDGSNKY | RFTISRDNSKNTLYLQMN | ---TMVRGVDY | WGQGTLVTVSS |
| | | | | | | RSLRLSCAASGFTFS | | GLEWVA | YADSVKG | SLRAEDTAVYYCAR | | |
| 32 | 6B10.1Vk | 6B10.1Vk | | | | DIQMTQSPSSLSASV | QASQDIYKS | WYQQRPGK | DASNLET | GVPSRFSGSGSGTDFTFT | QQYDNLPLT | FGGGTRVEIK |
| | | | | | | GDRVTITC | LN | APNLLIY | | ISSLQPEDFARYFC | | |
| 56 | Germline | Germline | VkO8/O18 | | Jk4 | DIQMTQSPSSLSASV | QASQDISNY | WYQQKPGK | DASNLET | GVPSRFSGSGSGTDFTFT | QQYDNLPLT | FGGGTKVEIK |
| | | | | | | GDRVTITC | LN | APKLLIY | | ISSLQPEDIATYYC | | |
| 34 | 3C1VH | 3C1VH | | | | QVQLVESGGGVVQPG | SYGMH | WVRQAPGK | IISYDGSNKY | RFTISRDNSKNTLYLQMN | GGRDYYYAMDV | WGQGTTVTVSS |
| | | | | | | RSLRLSCAASGFTFS | | GLEWVA | YADSVKG | SLKTEDTAVYYCAR | | |
| 57 | Germline | Germline | VH3-30*01 | D3-16 | JH6 | QVQLVESGGGVVQPG | SYGMH | WVRQAPGK | VISYDGSNKY | RFTISRDNSKNTLYLQMN | GG--YYYGMDV | WGQGTTVTVSS |
| | | | | | | RSLRLSCAASGFTFS | | GLEWVA | YADSVKG | SLRAEDTAVYYCAR | | |
| 36 | 3C1Vk | 3C1Vk | | | | DIQMTQSPSSLSASV | RASQNIYSY | WFQQKPGK | TASSLQS | GVPSRFSGSGSGTDFTLT | QQGYSTPLT | FGGGTKVDIK |
| | | | | | | GDRVTITC | LN | APKLLIY | | ISSLQPEDFATYYC | | |
| 58 | Germline | Germline | VkO2/O12 | | Jk4 | DIQMTQSPSSLSASV | RASQSISSY | WYQQKPGK | AASSLQS | GVPSRFSGSGSGTDFTLT | QQSYSTPLT | FGGGTKVEIK |
| | | | | | | GDRVTITC | LN | APKLLIY | | ISSLQPEDFATYYC | | |
| 38 | 6A6.2VH | 6A6.2VH | | | | EVQLLESGGGLVQPG | SYAMS | WVRQAPGK | TISGGGHSTY | RFTISRDNSKNTLYLQMN | IAPAGPHFDY | WGQGTLVTVSS |
| | | | | | | GSLRLSCAASGFTFS | | GLEWVS | YADSVKG | SLRAEDTAVYYCAR | | |
| 59 | Germline | Germline | VH3-23 | D6-13 | JH4 | EVQLLESGGGLVQPG | SYAMS | WVRQAPGK | AISGSGGSTY | RFTISRDNSKNTLYLQMN | IAAAG-YFDY | WGQGTLVTVSS |
| | | | | | | GSLRLSCAASGFTFS | | GLEWVS | YADSVKG | SLRAEDTAVYYCAK | | |
| 40 | 6A6.2VL | 6A6.2VL | | | | NFMLTQPHSVSESPG | TRSSGSIAS | WYQQRPGS | EHNQRPS | GVPDRFSGSIDSSSSSAS | QFYDRNSHWV | FGGGTKLTVL |
| | | | | | | KTVTFSC | NFVQ | SPTTVIY | | LTISGLKTEDEADYYC | | |
| 60 | Germline | Germline | V1-22 | | JL3b | NFMLTQPHSVSESPG | TRSSGSIAS | WYQQRPGS | EDNQRPS | GVPDRFSGSIDSSSNSAS | QSYDSSN-WV | FGGGTKLTVL |
| | | | | | | KTVTISC | NYVQ | SPTTVIY | | LTISGLKTEDEADYYC | | |

### DEFINITIONS

Unless otherwise defined, scientific and technical terms used herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art.

Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (3rd ed., Cold Spring Harbor Laboratory Press, Cold Spring Harbor, N.Y. (2001)), which is incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

An antagonist or inhibitor may be a polypeptide, nucleic acid, carbohydrate, lipid, small molecular weight compound, an oligonucleotide, an oligopeptide, RNA interference (RNAi), antisense, a recombinant protein, an antibody, or fragments thereof or conjugates or fusion proteins thereof. For a review of RNAi see Milhavet O, Gary D S, Mattson M P. (Pharmacol Rev. 2003 December; 55(4):629-48. Review) and antisense (see Opalinska J B, Gewirtz A M. (Sci STKE. 2003 Oct. 28; 2003 (206):pe47.)

A compound refers to any small molecular weight compound with a molecular weight of less than about 2000 Daltons.

The term “CD105” refers to the molecule that is CD105 protein, also known as CD105 antigen, END, Endoglin, FLJ41744, HHT1, ORW and ORW1.

The terms “neutralizing” or “inhibits” when referring to a targeted binding agent, such as an antibody, relates to the ability of an antibody to eliminate, reduce, or significantly reduce, the activity of a target antigen. Accordingly, a “neutralizing” anti-CD105 antibody of the invention is capable of eliminating or significantly reducing the activity of CD105. A neutralizing CD105 antibody may, for example, act by blocking the binding of a CD105 ligand to CD105, such as, for example, TGF-β. By blocking this binding, CD105 signal-mediated activity is significantly, or completely, eliminated. Ideally, a neutralizing antibody against CD105 inhibits tumor angiogenesis and/or cellular proliferation.

An “antagonist of the biological activity of CD105” is capable of eliminating, reducing or significantly reducing the activity of CD105. An “antagonist of the biological activity of CD105” is capable of eliminating, reducing or significantly reducing CD105 signaling. An “antagonist of the biological activity of CD105” may eliminate or significantly reduce tumor angiogenesis and/or cellular proliferation.

“Reducing CD105 signaling” encompasses a reduction of CD105 signaling by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% in comparison with the level of signaling in the absence of a targeted binding agent, antibody or antagonist of the invention.

An “optimized” sequence is an antibody sequence (variable heavy or light chain of any of the antibodies described herein) that has been mutated such that the non-germline sequence is mutated back at one or more residues to the germline sequence, and can further include the removal of structural liabilities from the sequence such as glycosylation sites or unpaired cysteines.

The term “polypeptide” is used herein as a generic term to refer to native protein, fragments, or analogs of a polypeptide sequence. Hence, native protein, fragments, and analogs are species of the polypeptide genus. Preferred polypeptides in accordance with the invention comprise the human heavy chain immunoglobulin molecules and the human kappa light chain immunoglobulin molecules, as well as antibody molecules formed by combinations comprising the heavy chain immunoglobulin molecules with light chain immunoglobulin molecules, such as the kappa or lambda light chain immunoglobulin molecules, and vice versa, as well as fragments and analogs thereof. Preferred polypeptides in accordance with the invention may also comprise solely the human heavy chain immunoglobulin molecules or fragments thereof.

The terms “native” or “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “operably linked” as used herein refers to positions of components so described that are in a relationship permitting them to function in their intended manner. For example, a control sequence “operably linked” to a coding sequence is connected in such a way that expression of the coding sequence is achieved under conditions compatible with the control sequences.

The term “polynucleotide” as referred to herein means a polymeric form of nucleotides of at least 10 bases in length, either ribonucleotides or deoxynucleotides or a modified form of either type of nucleotide, or RNA-DNA hetero-duplexes. The term includes single and double stranded forms of DNA.

The term “oligonucleotide” referred to herein includes naturally occurring, and modified nucleotides linked together by naturally occurring, and non-naturally occurring linkages. Oligonucleotides are a polynucleotide subset generally comprising a length of 200 bases or fewer. Preferably, oligonucleotides are 10 to 60 bases in length and most preferably 12, 13, 14, 15, 16, 17, 18, 19, or 20 to 40 bases in length. Oligonucleotides are usually single stranded, e.g. for probes; although oligonucleotides may be double stranded, e.g. for use in the construction of a gene mutant. Oligonucleotides can be either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phosphoraniladate, phosphoroamidate, and the like. See e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990), the disclosures of which are hereby incorporated by reference. An oligonucleotide can include a label for detection, if desired.

The term “selectively hybridise” referred to herein means to detectably and specifically bind. Polynucleotides, oligonucleotides and fragments thereof selectively hybridise to nucleic acid strands under hybridisation and wash conditions that minimise appreciable amounts of detectable binding to nonspecific nucleic acids. High stringency conditions can be used to achieve selective hybridisation conditions as known in the art and discussed herein. Generally, the nucleic acid sequence homology between the polynucleotides, oligonucleotides, or antibody fragments and a nucleic acid sequence of interest will be at least 80%, and more typically with preferably increasing homologies of at least 85%, 90%, 95%, 99%, and 100%.

Stringent hybridization conditions include, but are not limited to, hybridization to filter-bound DNA in 6× sodium chloride/sodium citrate (SSC) (0.9 M NaCl/90 mM NaCitrate, pH 7.0) at about 45° C. followed by one or more washes in 0.2×SSC/0.1% SDS at about 50-65° C., highly stringent conditions such as hybridization to filter-bound DNA in 6×SSC at about 45° C. followed by one or more washes in 0.1×SSC/0.2% SDS at about 60° C., or any other stringent hybridization conditions known to those skilled in the art (see, for example, Ausubel, F. M. et al., eds. 1989 Current Protocols in Molecular Biology, vol. 1, Green Publishing Associates, Inc. and John Wiley and Sons, Inc., NY at pages 6.3.1 to 6.3.6 and 2.10.3). Two amino acid sequences are “homologous” if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least about 30 amino acids in length) are homologous, as this term is used herein, if they have an alignment score of more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program. It should be appreciated that there can be differing regions of homology within two orthologous sequences. For example, the functional sites of mouse and human orthologues may have a higher degree of homology than non-functional regions.

The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence.

In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or amino acid residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more preferably at least 99 percent sequence identity, as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology—A Synthesis (2^{nd} Edition, E.S. Golub and D.R. Gren, Eds., Sinauer Associates, Sunderland, Mass. (1991)), which is incorporated herein by reference. Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Similarly, unless specified otherwise, the left-hand end of single-stranded polynucleotide sequences is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences”.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity. Preferably, residue positions that are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, glutamic-aspartic, and asparagine-glutamine.

As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99% sequence identity to the antibodies or immunoglobulin molecules described herein. In particular, conservative amino acid replacements are contemplated. Conservative replacements are those that take place within a family of amino acids that have related side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are an aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding function or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the antibodies described herein.

Glutaminyl and asparaginyl residues are frequently deamidated to the corresponding glutamyl and aspartyl residues, respectively. These residues are deamidated under neutral or basic conditions. The deamidated form of these residues falls within the scope of this invention.

In general, cysteine residues in proteins are either engaged in cysteine-cysteine disulfide bonds or sterically protected from the disulfide bond formation when they are a part of folded protein region. Disulfide bond formation in proteins is a complex process, which is determined by the redox potential of the environment and specialized thiol-disulfide exchanging enzymes (Creighton, Methods Enzymol. 107, 305-329, 1984; Houee-Levin, Methods Enzymol. 353, 35-44, 2002). When a cysteine residue does not have a pair in protein structure and is not sterically protected by folding, it can form a disulfide bond with a free cysteine from solution in a process known as disulfide shuffling. In another process known as disulfide scrambling, free cysteines may also interfere with naturally occurring disulfide bonds (such as those present in antibody structures) and lead to low binding, low biological activity and/or low stability.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various mutations of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W.H. Freeman and Company, New York (1984)); Introduction to Protein Structure (C. Branden and J. Tooze, eds., Garland Publishing, New York, N.Y. (1991)); and Thornton et at. Nature 354:105 (1991), which are each incorporated herein by reference.

Additionally, such methods may be used to make amino acid substitutions or deletions of one or more variable region cysteine residues participating in an intrachain disulfide bond to generate antibody molecules lacking one or more intrachain disulfide bonds.

The term “CDR region” or “CDR” is intended to indicate the hypervariable regions of the heavy and light chains of an antibody which confer antigen-binding specificity to the antibody. CDRs may be defined according to the Kabat system (Kabat, E. A. et al. (1991) Sequences of Proteins of Immunological Interest, 5th Edition. US Department of Health and Human Services, Public Service, NIH, Washington), and later editions. An antibody typically contains 3 heavy chain CDRs and 3 light chain CDRs. The term CDR or CDRs is used here in order to indicate, according to the case, one of these regions or several, or even the whole, of these regions which contain the majority of the amino acid residues responsible for the binding by affinity of the antibody for the antigen or the epitope which it recognises.

The third CDR of the heavy chain (HCDR3) has a greater size variability (greater diversity essentially due to the mechanisms of arrangement of the genes which give rise to it). It may be as short as 2 amino acids although the longest size known is 26. CDR length may also vary according to the length that can be accommodated by the particular underlying framework. Functionally, HCDR3 plays a role in part in the determination of the specificity of the antibody (Segal et al., PNAS, 71:4298-4302, 1974, Amit et al., Science, 233:747-753, 1986, Chothia et al., J. Mol. Biol., 196:901-917, 1987, Chothia et al., Nature, 342:877-883, 1989, Caton et al., J. Immunol., 144:1965-1968, 1990, Sharon et al., PNAS, 87:4814-4817, 1990, Sharon et al., J. Immunol., 144:4863-4869, 1990, Kabat et al., J. Immunol., 147:1709-1719, 1991).

The term a “set of CDRs” referred to herein comprises CDR1, CDR2 and CDR3. Thus, a set of HCDRs refers to HCDR1, HCDR2 and HCDR3, and a set of LCDRs refers to LCDR1, LCDR2 and LCDR3.

Variants of the VH and VL domains and CDRs of the present invention, including those for which amino acid sequences are set out herein, and which can be employed in targeting agents and antibodies for CD105 can be obtained by means of methods of sequence alteration or mutation and screening for antigen targeting with desired characteristics. Examples of desired characteristics include but are not limited to: increased binding affinity for antigen relative to known antibodies which are specific for the antigen; increased neutralisation of an antigen activity relative to known antibodies which are specific for the antigen if the activity is known; specified competitive ability with a known antibody or ligand to the antigen at a specific molar ratio; ability to immunoprecipitate ligand-receptor complex; ability to bind to a specified epitope; linear epitope, e.g. peptide sequence identified using peptide-binding scan, e.g. using peptides screened in linear and/or constrained conformation; conformational epitope, formed by non-continuous residues; ability to modulate a new biological activity of CD105, or downstream molecule; ability to bind and/or neutralise CD105 and/or for any other desired property.

The techniques required to make substitutions within amino acid sequences of CDRs, antibody VH or VL domains and antigen binding sites are available in the art. Variants of antibody molecules disclosed herein may be produced and used in the present invention. Following the lead of computational chemistry in applying multivariate data analysis techniques to the structure/property-activity relationships (Wold, et al. Multivariate data analysis in chemistry. Chemometrics Mathematics and Statistics in Chemistry (Ed.: B. Kowalski), D. Reidel Publishing Company, Dordrecht, Holland, 1984) quantitative activity-property relationships of antibodies can be derived using well-known mathematical techniques, such as statistical regression, pattern recognition and classification (Norman et al. Applied Regression Analysis. Wiley-Interscience; 3rd edition (April 1998); Kandel, Abraham & Backer, Eric. Computer-Assisted Reasoning in Cluster Analysis. Prentice Hall PTR, (May 11, 1995); Krzanowski, Wojtek. Principles of Multivariate Analysis: A User's Perspective (Oxford Statistical Science Series, No 22 (Paper)). Oxford University Press; (December 2000); Witten, Ian H. & Frank, Eibe. Data Mining: Practical Machine Learning Tools and Techniques with Java Implementations. Morgan Kaufmann; (Oct. 11, 1999); Denison David G. T. (Editor), Christopher C. Holmes, Bani K. Mallick, Adrian F. M. Smith. Bayesian Methods for Nonlinear Classification and Regression (Wiley Series in Probability and Statistics). John Wiley & Sons; (July 2002); Ghose, Arup K. & Viswanadhan, Vellarkad N. Combinatorial Library Design and Evaluation Principles, Software, Tools, and Applications in Drug Discovery). In some cases the properties of antibodies can be derived from empirical and theoretical models (for example, analysis of likely contact residues or calculated physicochemical property) of antibody sequence, functional and three-dimensional structures and these properties can be considered singly and in combination.

An antibody antigen-binding site composed of a VH domain and a VL domain is typically formed by six loops of polypeptide: three from the light chain variable domain (VL) and three from the heavy chain variable domain (VH). Analysis of antibodies of known atomic structure has elucidated relationships between the sequence and three-dimensional structure of antibody combining sites. These relationships imply that, except for the third region (loop) in VH domains, binding site loops have one of a small number of main-chain conformations: canonical structures. The canonical structure formed in a particular loop has been shown to be determined by its size and the presence of certain residues at key sites in both the loop and in framework regions.

This study of sequence-structure relationship can be used for prediction of those residues in an antibody of known sequence, but of an unknown three-dimensional structure, which are important in maintaining the three-dimensional structure of its CDR loops and hence maintain binding specificity. These predictions can be backed up by comparison of the predictions to the output from lead optimisation experiments. In a structural approach, a model can be created of the antibody molecule using any freely available or commercial package, such as WAM. A protein visualisation and analysis software package, such as Insight II (Accelrys, Inc.) or Deep View may then be used to evaluate possible substitutions at each position in the CDR. This information may then be used to make substitutions likely to have a minimal or beneficial effect on activity or confer other desirable properties.

The term “polypeptide fragment” as used herein refers to a polypeptide that has an amino-terminal and/or carboxy-terminal deletion, but where the remaining amino acid sequence is identical to the corresponding positions in the naturally-occurring sequence deduced, for example, from a full-length cDNA sequence. Fragments typically are at least 5, 6, 8 or 10 amino acids long, preferably at least 14 amino acids long, more preferably at least 20 amino acids long, usually at least 50 amino acids long, and even more preferably at least 70 amino acids long. The term “analog” as used herein refers to polypeptides which are comprised of a segment of at least 25 amino acids that has substantial identity to a portion of a deduced amino acid sequence and which has at least one of the following properties: (1) specific binding to CD105, under suitable binding conditions, (2) ability to block appropriate TGFβ/CD105 binding, or (3) ability to inhibit CD105 activity. Typically, polypeptide analogs comprise a conservative amino acid substitution (or addition or deletion) with respect to the naturally-occurring sequence. Analogs typically are at least 20 amino acids long, preferably at least 50 amino acids long or longer, and can often be as long as a full-length naturally-occurring polypeptide.

Peptide analogs are commonly used in the pharmaceutical industry as non-peptide drugs with properties analogous to those of the template peptide. These types of non-peptide compound are termed “peptide mimetics” or “peptidomimetics” (Fauchere, J. Adv. Drug Res. 15:29 (1986); Veber and Freidinger TINS p. 392 (1985); and Evans et al. J. Med. Chem. 30:1229 (1987), which are incorporated herein by reference). Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from the group consisting of: —CH₂NH—, —CH₂S—, —CH₂—CH₂—, —CH═CH—(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992), incorporated herein by reference); for example, by adding internal cysteine residues capable of forming intramolecular disulfide bridges which cyclize the peptide.

An antibody may be oligoclonal, a polyclonal antibody, a monoclonal antibody, a chimeric antibody, a CDR-grafted antibody, a multi-specific antibody, a bi-specific antibody, a catalytic antibody, a chimeric antibody, a humanized antibody, a fully human antibody, an anti-idiotypic antibody and antibodies that can be labeled in soluble or bound form as well as fragments, variants or derivatives thereof, either alone or in combination with other amino acid sequences provided by known techniques. An antibody may be from any species.

As used herein, the terms “antibody” and “antibodies” (immunoglobulins) encompass monoclonal antibodies (including full-length monoclonal antibodies), polyclonal antibodies, camelised antibodies and chimeric antibodies. As used herein, the term “antibody” or “antibodies” refers to a polypeptide or group of polypeptides that are comprised of at least one binding domain that is formed from the folding of polypeptide chains having three-dimensional binding spaces with internal surface shapes and charge distributions complementary to the features of an antigenic determinant of an antigen. chain. Native antibodies are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Light chains are classified as either lambda chains or kappa chains based on the amino acid sequence of the light chain constant region. The variable domain of a kappa light chain may also be denoted herein as VK. The term “variable region” may also be used to describe the variable domain of a heavy chain or light chain. Particular amino acid residues are believed to form an interface between the light and heavy chain variable domains. The variable regions of each light/heavy chain pair form an antibody binding site. Such antibodies may be derived from any mammal, including, but not limited to, humans, monkeys, pigs, horses, rabbits, dogs, cats, mice, etc.

The term “antibody” or “antibodies” includes binding fragments of the antibodies of the invention, exemplary fragments include single-chain Fvs (scFv), single-chain antibodies, single domain antibodies, domain antibodies, Fv fragments, Fab fragments, F(ab′) fragments, F(ab′)2 fragments, antibody fragments that exhibit the desired biological activity, disulfide-stabilised variable region (dsFv), dimeric variable region (Diabody), anti-idiotypic (anti-Id) antibodies (including, e.g., anti-Id antibodies to antibodies of the invention), intrabodies, linear antibodies, single-chain antibody molecules and multispecific antibodies formed from antibody fragments and epitope-binding fragments of any of the above. In particular, antibodies include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

Digestion of antibodies with the enzyme, papain, results in two identical antigen-binding fragments, known also as “Fab” fragments, and a “Fc” fragment, having no antigen-binding activity but having the ability to crystallize Digestion of antibodies with the enzyme, pepsin, results in the a F(ab′)₂ fragment in which the two arms of the antibody molecule remain linked and comprise two-antigen binding sites. The F(ab′)₂ fragment has the ability to crosslink antigen.

“Fv” when used herein refers to the minimum fragment of an antibody that retains both antigen-recognition and antigen-binding sites. This region consists of a dimer of one heavy and one light chain variable domain in tight, non-covalent or covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

“Fab” when used herein refers to a fragment of an antibody that comprises the constant domain of the light chain and the CH1 domain of the heavy chain.

“dAb” when used herein refers to a fragment of an antibody that is the smallest functional binding unit of a human antibodies. A “dAb” is a single domain antibody and comprises either the variable domain of an antibody heavy chain (VH domain) or the variable domain of an antibody light chain (VL domain). Each dAb contains three of the six naturally occurring CDRs (Ward et al., Binding activities of a repertoire of single immunoglobulin variable domains secreted from Escherichia coli. Nature 341, 544-546 (1989); Holt, et al., Domain antibodies: protein for therapy, Trends Biotechnol. 21, 484-49 (2003)). With molecular weights ranging from 11 to 15 kDa, they are four times smaller than a fragment antigen binding (Fab)2 and half the size of a single chain Fv (scFv) molecule.

“Camelid” when used herein refers to antibody molecules are composed of heavy-chain dimers which are devoid of light chains, but nevertheless have an extensive antigen-binding repertoire (Hamers-Casterman C, Atarhouch T, Muyldermans S, Robinson G, Hamers C, Songa E B, Bendahman N, Hamers R (1993) Naturally occurring antibodies devoid of light chains. Nature 363:446-448).

The term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

It has been shown that fragments of a whole antibody can perform the function of binding antigens. Examples of binding fragments are (Ward, E. S. et al., (1989) Nature 341, 544-546) the Fab fragment consisting of VL, VH, CL and CH1 domains; (McCafferty et al (1990) Nature, 348, 552-554) the Fd fragment consisting of the VH and CH1 domains; (Holt et al (2003) Trends in Biotechnology 21, 484-490) the Fv fragment consisting of the VL and VH domains of a single antibody; (iv) the dAb fragment (Ward, E. S. et al., Nature 341, 544-546 (1989), McCafferty et al (1990) Nature, 348, 552-554, Holt et al (2003) Trends in Biotechnology 21, 484-490], which consists of a VH or a VL domain; (v) isolated CDR regions; (vi) F(ab′)₂ fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, (1988) Science, 242, 423-426 Huston et al, (1988) PNAS USA, 85, 5879-5883); (viii) bispecific single chain Fv dimers (PCT/US92/09965) and (ix) “diabodies”, multivalent or multispecific fragments constructed by gene fusion (WO94/13804; Holliger, P. (1993) et al, Proc. Natl. Acad. Sci. USA 90 6444-6448). Fv, scFv or diabody molecules may be stabilised by the incorporation of disulphide bridges linking the VH and VL domains (Reiter, Y. et al, Nature Biotech, 14, 1239-1245, 1996). Minibodies comprising a scFv joined to a CH3 domain may also be made (Hu, S. et al, (1996) Cancer Res., 56, 3055-3061). Other examples of binding fragments are Fab′, which differs from Fab fragments by the addition of a few residues at the carboxyl terminus of the heavy chain CH1 domain, including one or more cysteines from the antibody hinge region, and Fab′-SH, which is a Fab′ fragment in which the cysteine residue(s) of the constant domains bear a free thiol group.

The term “variable” refers to the fact that certain portions of the variable domains differ extensively in sequence among antibodies and are responsible for the binding specificity of each particular antibody for its particular antigen. However, the variability is not evenly distributed through the variable domains of antibodies. It is concentrated in segments called Complementarity Determining Regions (CDRs) both in the light chain and the heavy chain variable domains. The more highly conserved portions of the variable domains are called the framework regions (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see, Kabat et al., Sequences of Proteins of Immunological Interest, 5th Ed. Public Health Service, National Institutes of Health, Bethesda, Md. (1991)). The constant domains are generally not involved directly in antigen binding, but may influence antigen binding affinity and may exhibit various effector functions, such as participation of the antibody in ADCC, CDC, and/or apoptosis.

The term “hypervariable region” when used herein refers to the amino acid residues of an antibody which are associated with its binding to antigen. The hypervariable regions encompass the amino acid residues of the “complementarity determining regions” or “CDRs” (e.g., residues 24-34 (L1), 50-56 (L2) and 89-97 (L3) of the light chain variable domain and residues 31-35 (H1), 50-65 (H2) and 95-102 (H3) of the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, J. Mol. Biol., 196:901-917 (1987)). “Framework” or “FR” residues are those variable domain residues flanking the CDRs. FR residues are present in chimeric, humanized, human, domain antibodies, diabodies, vaccibodies, linear antibodies, and bispecific antibodies.

As used herein, targeted binding agent, targeted binding protein, specific binding protein and like terms refer to an antibody, or binding fragment thereof that preferentially binds to a target site. In one embodiment, the targeted binding agent is specific for only one target site. In other embodiments, the targeted binding agent is specific for more than one target site. In one embodiment, the targeted binding agent may be a monoclonal antibody and the target site may be an epitope.

“Binding fragments” of an antibody are produced by recombinant DNA techniques, or by enzymatic or chemical cleavage of intact antibodies. Binding fragments include Fab, Fab′, F(ab′)₂, Fv, dAb and single-chain antibodies. An antibody other than a “bispecific” or “bifunctional” antibody is understood to have each of its binding sites identical. An antibody substantially inhibits adhesion of a receptor to a counter-receptor when an excess of antibody reduces the quantity of receptor bound to counter-receptor by at least about 20%, 40%, 60% or 80%, and more usually greater than about 85% (as measured in an in vitro competitive binding assay).

The term “epitope” includes any protein determinant capable of specific binding to an immunoglobulin or T-cell receptor. Epitopic determinants usually consist of chemically active surface groupings of molecules such as amino acids or sugar side chains and may, but not always, have specific three-dimensional structural characteristics, as well as specific charge characteristics. An antibody is said to specifically bind an antigen when the dissociation constant is ≦1 μM, preferably ≦100 nM and most preferably ≦10 nM.

The term “agent” is used herein to denote a chemical compound, a mixture of chemical compounds, a biological macromolecule, or an extract made from biological materials.

“Active” or “activity” in regard to a CD105 polypeptide refers to a portion of an CD105 polypeptide that has a biological or an immunological activity of a native CD105 polypeptide. “Biological” when used herein refers to a biological function that results from the activity of the native CD105 polypeptide. A preferred CD105 biological activity includes, for example, CD105 induced cell adhesion and invasion and/or angiogenesis and/or proliferation.

“Mammal” when used herein refers to any animal that is considered a mammal. Preferably, the mammal is human.

“Animal” when used herein encompasses animals considered a mammal. Preferably the animal is human.

The term “mAb” refers to monoclonal antibody.

“Liposome” when used herein refers to a small vesicle that may be useful for delivery of drugs that may include the CD105 polypeptide of the invention or antibodies to such an CD105 polypeptide to a mammal.

“Label” or “labeled” as used herein refers to the addition of a detectable moiety to a polypeptide, for example, a radiolabel, fluorescent label, enzymatic label chemiluminescent labeled or a biotinyl group. Radioisotopes or radionuclides may include ³H, ¹⁴C, ¹⁵N, ³⁵S, ⁹⁰Y, ⁹⁹Tc, ¹¹¹In, ¹²⁵I, ¹³¹I, fluorescent labels may include rhodamine, lanthanide phosphors or FITC and enzymatic labels may include horseradish peroxidase, β-galactosidase, luciferase, alkaline phosphatase.

Additional labels include, by way of illustration and not limitation: enzymes, such as glucose-6-phosphate dehydrogenase (“G6PDH”), alpha-D-galactosidase, glucose oxydase, glucose amylase, carbonic anhydrase, acetylcholinesterase, lysozyme, malate dehydrogenase and peroxidase; dyes; additional fluorescent labels or fluorescers include, such as fluorescein and its derivatives, fluorochrome, GFP (GFP for “Green Fluorescent Protein”), dansyl, umbelliferone, phycoerythrin, phycocyanin, allophycocyanin, o-phthaldehyde, and fluorescamine; fluorophores such as lanthanide cryptates and chelates e.g. Europium etc (Perkin Elmer and Cis Biointernational); chemoluminescent labels or chemiluminescers, such as isoluminol, luminol and the dioxetanes; sensitisers; coenzymes; enzyme substrates; particles, such as latex or carbon particles; metal sol; crystallite; liposomes; cells, etc., which may be further labelled with a dye, catalyst or other detectable group; molecules such as biotin, digoxygenin or 5-bromodeoxyuridine; toxin moieties, such as for example a toxin moiety selected from a group of Pseudomonas exotoxin (PE or a cytotoxic fragment or mutant thereof), Diptheria toxin or a cytotoxic fragment or mutant thereof, a botulinum toxin A, B, C, D, E or F, ricin or a cytotoxic fragment thereof e.g. ricin A, abrin or a cytotoxic fragment thereof, saporin or a cytotoxic fragment thereof, pokeweed antiviral toxin or a cytotoxic fragment thereof and bryodin 1 or a cytotoxic fragment thereof.

The term “pharmaceutical agent or drug” as used herein refers to a chemical compound or composition capable of inducing a desired therapeutic effect when properly administered to a patient. Other chemistry terms herein are used according to conventional usage in the art, as exemplified by The McGraw-Hill Dictionary of Chemical Terms (Parker, S., Ed., McGraw-Hill, San Francisco (1985)), (incorporated herein by reference).

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term “patient” includes human and veterinary subjects.

“Antibody-dependent cell-mediated cytotoxicity” and “ADCC” refer to a cell-mediated reaction in which non-specific cytotoxic cells that express Ig Fc receptors (FcRs) (e.g. Natural Killer (NK) cells, monocytes, neutrophils, and macrophages) recognise bound antibody on a target cell and subsequently cause lysis of the target cell. The primary cells for mediating ADCC, NK cells, express FcγRIII only, whereas monocytes express FcγRI, FcγRII and FcγRIII. FcRs expression on hematopoietic cells is summarised in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362, or U.S. Pat. No. 5,821,337 can be performed. Useful effector cells for such assays include peripheral blood mononuclear cells (PBMC) and Natural Killer (NK) cells. Alternatively, or additionally, ADCC activity of the molecule of interest can be assessed in vivo, e.g., in an animal model such as that disclosed in Clynes et al. PNAS (USA) 95:652-656 (1988). “Complement dependent cytotoxicity” and “CDC” refer to the mechanism by which antibodies carry out their cell-killing function. It is initiated by the binding of C1q, a constituent of the first component of complement, to the Fc domain of Igs, IgG or IgM, which are in complex with antigen (Hughs-Jones, N. C., and B. Gardner. 1979. Mol. Immunol. 16:697). C1q is a large, structurally complex glycoprotein of ˜410 kDa present in human serum at a concentration of 70 μg/ml (Cooper, N. R. 1985. Adv. Immunol. 37:151). Together with two serine proteases, C1r and C1s, C1q forms the complex C1, the first component of complement. At least two of the N-terminal globular heads of C1q must be bound to the Fc of Igs for C1 activation, hence for initiation of the complement cascade (Cooper, N. R. 1985. Adv. Immunol. 37:151).

The term “antibody half-life” as used herein means a pharmacokinetic property of an antibody that is a measure of the mean survival time of antibody molecules following their administration. Antibody half-life can be expressed as the time required to eliminate 50 percent of a known quantity of immunoglobulin from the patient's body or a specific compartment thereof, for example, as measured in serum or plasma, i.e., circulating half-life, or in other tissues. Half-life may vary from one immunoglobulin or class of immunoglobulin to another. In general, an increase in antibody half-life results in an increase in mean residence time (MRT) in circulation for the antibody administered.

The term “isotype” refers to the classification of an antibody's heavy or light chain constant region. The constant domains of antibodies are not involved in binding to antigen, but exhibit various effector functions. Depending on the amino acid sequence of the heavy chain constant region, a given human antibody or immunoglobulin can be assigned to one of five major classes of immunoglobulins: IgA, IgD, IgE, IgG, and IgM. Several of these classes may be further divided into subclasses (isotypes), e.g., IgG1 (gamma 1), IgG2 (gamma 2), IgG3 (gamma 3), and IgG4 (gamma 4), and IgA1 and IgA2. The heavy chain constant regions that correspond to the different classes of immunoglobulins are called α, δ, ε, γ, and μ, respectively. The structures and three-dimensional configurations of different classes of immunoglobulins are well-known. Of the various human immunoglobulin classes, only human IgG1, IgG2, IgG3, IgG4, and IgM are known to activate complement. Human IgG1 and IgG3 are known to mediate in humans. Human light chain constant regions may be classified into two major classes, kappa and lambda.

If desired, the isotype of an antibody that specifically binds CD105 can be switched, for example to take advantage of a biological property of a different isotype. For example, in some circumstances it can be desirable in connection with the generation of antibodies as therapeutic antibodies against CD105 that the antibodies be capable of fixing complement and participating in complement-dependent cytotoxicity (CDC). There are a number of isotypes of antibodies that are capable of the same, including, without limitation, the following: murine IgM, murine IgG2a, murine IgG2b, murine IgG3, human IgM, human IgA, human IgG1, and human IgG3. In other embodiments it can be desirable in connection with the generation of antibodies as therapeutic antibodies against CD105 that the antibodies be capable of binding Fc receptors on effector cells and participating in antibody-dependent cytotoxicity (ADCC). There are a number of isotypes of antibodies that are capable of the same, including, without limitation, the following: murine IgG2a, murine IgG2b, murine IgG3, human IgG1, and human IgG3. It will be appreciated that antibodies that are generated need not initially possess such an isotype but, rather, the antibody as generated can possess any isotype and the antibody can be isotype switched thereafter using conventional techniques that are well known in the art. Such techniques include the use of direct recombinant techniques (see e.g., U.S. Pat. No. 4,816,397), cell-cell fusion techniques (see e.g., U.S. Pat. Nos. 5,916,771 and 6,207,418), among others.

By way of example, the anti-CD105 antibodies discussed herein are fully human antibodies. If an antibody possessed desired binding to CD105, it could be readily isotype switched to generate a human IgM, human IgG1, or human IgG3 isotype, while still possessing the same variable region (which defines the antibody's specificity and some of its affinity). Such molecule would then be capable of fixing complement and participating in CDC and/or be capable of binding to Fc receptors on effector cells and participating in ADCC.

“Whole blood assays” use unfractionated blood as a source of natural effectors. Blood contains complement in the plasma, together with FcR-expressing cellular effectors, such as polymorphonuclear cells (PMNs) and mononuclear cells (MNCs). Thus, whole blood assays allow simultaneous evaluation of the synergy of both ADCC and CDC effector mechanisms in vitro.

A “therapeutically effective” amount as used herein is an amount that provides some improvement or benefit to the subject. Stated in another way, a “therapeutically effective” amount is an amount that provides some alleviation, mitigation, and/or decrease in at least one clinical symptom. Clinical symptoms associated with the disorders that can be treated by the methods of the invention are well-known to those skilled in the art. Further, those skilled in the art will appreciate that the therapeutic effects need not be complete or curative, as long as some benefit is provided to the subject.

The term “and/or” as used herein is to be taken as specific disclosure of each of the two specified features or components with or without the other. For example “A and/or B” is to be taken as specific disclosure of each of (i) A, (ii) B and (iii) A and B, just as if each is set out individually herein.

### Antibody Structure

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site.

Thus, an intact antibody has two binding sites. Except in bifunctional or bispecific antibodies, the two binding sites are the same.

The chains all exhibit the same general structure of relatively conserved framework regions (FR) joined by three hyper variable regions, also called CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

A bispecific or bifunctional antibody is an artificial hybrid antibody having two different heavy/light chain pairs and two different binding sites. In one example, a bispecific antibody of the present invention is an antibody that has binding specificity for at least two different CD105 epitopes. Since a number of the CD105 targeted binding agents of the invention have different epitopes or have partial or overlapping epitopes it is contemplated that a bispecific antibody of the invention can include any combination of the CD105 targeted binding agents having different or overlapping epitopes. For example, 6A6 and 6B10 have a different epitope than 4D4 and 10C9. In one example the bispecific antibody has the hypervariable region, or a region having at least 50, 60, 70, 80, or 90% homology thereto, of 6A6 or 6B10 and variable or hypervariable region of 4D4 or 10C9, or a region having at least 50, 60, 70, 80, or 90% homology thereto.

Bispecific antibodies can be produced by a variety of methods including fusion of hybridomas or linking of Fab′ fragments. See, e.g., Songsivilai & Lachmann Clin. Exp. Immunol. 79: 315-321 (1990), Kostelny et al. J. Immunol. 148:1547-1553 (1992). Bispecific antibodies do not exist in the form of fragments having a single binding site (e.g., Fab, Fab′, and Fv). Typically, a VH domain is paired with a VL domain to provide an antibody antigen-binding site, although a VH or VL domain alone may be used to bind antigen. The VH domain (see Table 2) may be paired with the VL domain (see Table 2), so that an antibody antigen-binding site is formed comprising both the VH and VL domains.

Typically, bispecific antibodies are antibodies that have binding specificities for at least two different epitopes. Exemplary bispecific antibodies may bind to two different epitopes of the CD105 protein. Other such antibodies may combine a CD105 binding site with a binding site for another protein. Alternatively, an anti-CD105 arm may be combined with an arm which binds to a triggering molecule on a leukocyte such as a T-cell receptor molecule (e.g. CD3), or Fc receptors for IgG (FcγR), such as FcγRI (CD64), FcγRII (CD32) and FcγRIII (CD16), so as to focus and localize cellular defense mechanisms to the CD105-expressing cell. Bispecific antibodies may also be used to localize cytotoxic agents to cells which express CD105. These antibodies possess a CD105-binding arm and an arm which binds the cytotoxic agent (e.g. saporin, anti-interferon-α, vinca alkaloid, ricin A chain, methotrexate or radioactive isotope hapten). Bispecific antibodies can be prepared as full length antibodies or antibody fragments (e.g. F(ab′)₂ bispecific antibodies). Methods for making bispecific antibodies are known in the art. (See, for example, Millstein et al., Nature, 305:537-539 (1983); Traunecker et al., EMBO J., 10:3655-3659 (1991); Suresh et al., Methods in Enzymology, 121:210 (1986); Kostelny et al., J. Immunol., 148(5):1547-1553 (1992); Hollinger et al., Proc. Natl Acad. Sci. USA, 90:6444-6448 (1993); Gruber et al., J. Immunol., 152:5368 (1994); U.S. Pat. Nos. 4,474,893; 4,714,681; 4,925,648; 5,573,920; 5,601,819; 5,731,168; 4,676,980; 5,897,861; 5,660,827; 5,811,267; 5,849,877; 5,948,647; 5,959,084; 6,106,833; 6,141,873 and 4,676,980, WO 94/04690; and WO 92/20373.)

Traditional production of full length bispecific antibodies is based on the co-expression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. Preferably, the fusion is with an Ig heavy chain constant domain, comprising at least part of the hinge, C_{H}2, and C_{H}3 regions. It is preferred to have the first heavy-chain constant region (C_{H}1) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant affect on the yield of the desired chain combination.

In one embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure may facilitate the separation of the desired bispecific compound from unwanted immunoglobulin chain combinations, as the presence of an immunoglobulin light chain in only one half of the bispecific molecule provides for a facile way of separation. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the C_{H}3 domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (U.S. Pat. No. 5,897,861). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med., 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a V_{H} connected to a V_{L} by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994) and U.S. Pat. Nos. 5,591,828; 4,946,778; 5,455,030; and 5,869,620.

### Human Antibodies and Humanization of Antibodies

Human antibodies avoid some of the problems associated with antibodies that possess murine or rat variable and/or constant regions. The presence of such murine or rat derived proteins can lead to the rapid clearance of the antibodies or can lead to the generation of an immune response against the antibody by a patient. In order to avoid the utilization of murine or rat derived antibodies, fully human antibodies can be generated through the introduction of functional human antibody loci into a rodent, other mammal or animal so that the rodent, other mammal or animal produces fully human antibodies.

One method for generating fully human antibodies is through the use of XenoMouse® strains of mice that have been engineered to contain up to but less than 1000 kb-sized germline configured fragments of the human heavy chain locus and kappa light chain locus. See Mendez et al. Nature Genetics 15:146-156 (1997) and Green and Jakobovits J. Exp. Med. 188:483-495 (1998). The XenoMouse® strains are available from Amgen, Inc. (Fremont, Calif., U.S.A).

Such mice, then, are capable of producing human immunoglobulin molecules and antibodies and are deficient in the production of murine immunoglobulin molecules and antibodies. Technologies utilised for achieving the same are disclosed in U.S. patent application Ser. No. 08/759,620, filed Dec. 3, 1996 and International Patent Application Nos. WO 98/24893, published Jun. 11, 1998 and WO 00/76310, published Dec. 21, 2000, the disclosures of which are hereby incorporated by reference. See also Mendez et al. Nature Genetics 15:146-156 (1997), the disclosure of which is hereby incorporated by reference.

The production of the XenoMouse® strains of mice is further discussed and delineated in U.S. patent application Ser. Nos. 07/466,008, filed Jan. 12, 1990, 07/610,515, filed Nov. 8, 1990, 07/919,297, filed Jul. 24, 1992, 07/922,649, filed Jul. 30, 1992, 08/031,801, filed Mar. 15, 1993, 08/112,848, filed Aug. 27, 1993, 08/234,145, filed Apr. 28, 1994, 08/376,279, filed Jan. 20, 1995, 08/430,938, filed Apr. 27, 1995, 08/464,584, filed Jun. 5, 1995, 08/464,582, filed Jun. 5, 1995, 08/463,191, filed Jun. 5, 1995, 08/462,837, filed Jun. 5, 1995, 08/486,853, filed Jun. 5, 1995, 08/486,857, filed Jun. 5, 1995, 08/486,859, filed Jun. 5, 1995, 08/462,513, filed Jun. 5, 1995, 08/724,752, filed Oct. 2, 1996, 08/759,620, filed Dec. 3, 1996, U.S. Publication 2003/0093820, filed Nov. 30, 2001 and U.S. Pat. Nos. 6,162,963, 6,150,584, 6,114,598, 6,075,181, and 5,939,598 and Japanese Patent Nos. 3 068 180 B2, 3 068 506 B2, and 3 068 507 B2. See also European Patent No., EP 0 463 151 B1, grant published Jun. 12, 1996, International Patent Application No., WO 94/02602, published Feb. 3, 1994, International Patent Application No., WO 96/34096, published Oct. 31, 1996, WO 98/24893, published Jun. 11, 1998, WO 00/76310, published Dec. 21, 2000. The disclosures of each of the above-cited patents, applications, and references are hereby incorporated by reference in their entirety.

In an alternative approach, others, including GenPharm International, Inc., have utilised a “minilocus” approach. In the minilocus approach, an exogenous Ig locus is mimicked through the inclusion of pieces (individual genes) from the Ig locus. Thus, one or more V_{H} genes, one or more D_{H} genes, one or more J_{H} genes, a mu constant region, and usually a second constant region (preferably a gamma constant region) are formed into a construct for insertion into an animal. This approach is described in U.S. Pat. No. 5,545,807 to Surani et al. and U.S. Pat. Nos. 5,545,806, 5,625,825, 5,625,126, 5,633,425, 5,661,016, 5,770,429, 5,789,650, 5,814,318, 5,877,397, 5,874,299, and 6,255,458 each to Lonberg and Kay, U.S. Pat. Nos. 5,591,669 and 6,023.010 to Krimpenfort and Berns, U.S. Pat. Nos. 5,612,205, 5,721,367, and 5,789,215 to Berns et al., and U.S. Pat. No. 5,643,763 to Choi and Dunn, and GenPharm International U.S. patent application Ser. No. 07/574,748, filed Aug. 29, 1990, 07/575,962, filed Aug. 31, 1990, 07/810,279, filed Dec. 17, 1991, 07/853,408, filed Mar. 18, 1992, 07/904,068, filed Jun. 23, 1992, 07/990,860, filed Dec. 16, 1992, 08/053,131, filed Apr. 26, 1993, 08/096,762, filed Jul. 22, 1993, 08/155,301, filed Nov. 18, 1993, 08/161,739, filed Dec. 3, 1993, 08/165,699, filed Dec. 10, 1993, 08/209,741, filed Mar. 9, 1994, the disclosures of which are hereby incorporated by reference. See also European Patent No. 0 546 073 B1, International Patent Application Nos. WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175, the disclosures of which are hereby incorporated by reference in their entirety. See further Taylor et al., 1992, Chen et al., 1993, Tuaillon et al., 1993, Choi et al., 1993, Lonberg et al., (1994), Taylor et al., (1994), and Tuaillon et al., (1995), Fishwild et al., (1996), the disclosures of which are hereby incorporated by reference in their entirety.

Kirin has also demonstrated the generation of human antibodies from mice in which, through microcell fusion, large pieces of chromosomes, or entire chromosomes, have been introduced. See European Patent Application Nos. 773 288 and 843 961, the disclosures of which are hereby incorporated by reference. Additionally, KM™-mice, which are the result of cross-breeding of Kirin's Tc mice with Medarex's minilocus (Humab) mice have been generated. These mice possess the human IgH transchromosome of the Kirin mice and the kappa chain transgene of the Genpharm mice (Ishida et al., Cloning Stem Cells, (2002) 4:91-102).

Human antibodies can also be derived by in vitro methods. Suitable examples include but are not limited to phage display (Medimmune, Morphosys, Dyax, Biosite/Medarex, Xoma, Symphogen, Alexion (formerly Proliferon), Affimed) ribosome display (Medimmune), yeast display, and the like.

### Preparation of Antibodies

Antibodies, as described herein, were prepared through the utilization of the XenoMouse® technology, as described below. Such mice are capable of producing human immunoglobulin molecules and antibodies and are deficient in the production of murine immunoglobulin molecules and antibodies. Technologies utilised for achieving the same are disclosed in the patents, applications, and references disclosed in the background section herein. In particular, however, a preferred embodiment of transgenic production of mice and antibodies therefrom is disclosed in U.S. patent application Ser. No. 08/759,620, filed Dec. 3, 1996 and International Patent Application Nos. WO 98/24893, published Jun. 11, 1998 and WO 00/76310, published Dec. 21, 2000, the disclosures of which are hereby incorporated by reference. See also Mendez et al. Nature Genetics 15:146-156 (1997), the disclosure of which is hereby incorporated by reference.

Through the use of such technology, fully human monoclonal antibodies to a variety of antigens have been produced. Essentially, XenoMouse® lines of mice are immunised with an antigen of interest (e.g. CD105), lymphatic cells (such as B-cells) are recovered from the hyper-immunised mice, and the recovered lymphocytes are fused with a myeloid-type cell line to prepare immortal hybridoma cell lines. These hybridoma cell lines are screened and selected to identify hybridoma cell lines that produced antibodies specific to the antigen of interest. Provided herein are methods for the production of multiple hybridoma cell lines that produce antibodies specific to CD105. Further, provided herein are characterisation of the antibodies produced by such cell lines, including nucleotide and amino acid sequence analyses of the heavy and light chains of such antibodies.

Alternatively, instead of being fused to myeloma cells to generate hybridomas, B cells can be directly assayed. For example, CD19+B cells can be isolated from hyperimmune XenoMouse® mice and allowed to proliferate and differentiate into antibody-secreting plasma cells. Antibodies from the cell supernatants are then screened by ELISA for reactivity against the CD105 immunogen. The supernatants might also be screened for immunoreactivity against fragments of CD105 to further map the different antibodies for binding to domains of functional interest on CD105. The antibodies may also be screened other related human endoglycosidases and against the rat, the mouse, and non-human primate, such as Cynomolgus monkey, orthologues of CD105, the last to determine species cross-reactivity. B cells from wells containing antibodies of interest may be immortalised by various methods including fusion to make hybridomas either from individual or from pooled wells, or by infection with EBV or transfection by known immortalising genes and then plating in suitable medium. Alternatively, single plasma cells secreting antibodies with the desired specificities are then isolated using an CD105-specific hemolytic plaque assay (see for example Babcook et al., Proc. Natl. Acad. Sci. USA 93:7843-48 (1996)). Cells targeted for lysis are preferably sheep red blood cells (SRBCs) coated with the CD105 antigen.

In the presence of a B-cell culture containing plasma cells secreting the immunoglobulin of interest and complement, the formation of a plaque indicates specific CD105-mediated lysis of the sheep red blood cells surrounding the plasma cell of interest. The single antigen-specific plasma cell in the center of the plaque can be isolated and the genetic information that encodes the specificity of the antibody is isolated from the single plasma cell. Using reverse-transcription followed by PCR (RT-PCR), the DNA encoding the heavy and light chain variable regions of the antibody can be cloned. Such cloned DNA can then be further inserted into a suitable expression vector, preferably a vector cassette such as a pcDNA, more preferably such a pcDNA vector containing the constant domains of immunoglobulin heavy and light chain. The generated vector can then be transfected into host cells, e.g., HEK293 cells, CHO cells, and cultured in conventional nutrient media modified as appropriate for inducing transcription, selecting transformants, or amplifying the genes encoding the desired sequences.

As will be appreciated, antibodies that specifically bind CD105 can be expressed in cell lines other than hybridoma cell lines. Sequences encoding particular antibodies can be used to transform a suitable mammalian host cell. Transformation can be by any known method for introducing polynucleotides into a host cell, including, for example packaging the polynucleotide in a virus (or into a viral vector) and transducing a host cell with the virus (or vector) or by transfection procedures known in the art, as exemplified by U.S. Pat. Nos. 4,399,216, 4,912,040, 4,740,461, and 4,959,455 (which patents are hereby incorporated herein by reference). The transformation procedure used depends upon the host to be transformed. Methods for introducing heterologous polynucleotides into mammalian cells are well known in the art and include dextran-mediated transfection, calcium phosphate precipitation, polybrene mediated transfection, protoplast fusion, electroporation, encapsulation of the polynucleotide(s) in liposomes, and direct microinjection of the DNA into nuclei.

Mammalian cell lines available as hosts for expression are well known in the art and include many immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), human epithelial kidney 293 cells, and a number of other cell lines. Cell lines of particular preference are selected through determining which cell lines have high expression levels and produce antibodies with constitutive CD105 binding properties.

In the cell-cell fusion technique, a myeloma, CHO cell or other cell line is prepared that possesses a heavy chain with any desired isotype and another myeloma, CHO cell or other cell line is prepared that possesses the light chain. Such cells can, thereafter, be fused and a cell line expressing an intact antibody can be isolated.

Accordingly, as antibody candidates are generated that meet desired “structural” attributes as discussed above, they can generally be provided with at least certain of the desired “functional” attributes through isotype switching.

### Therapeutic Administration and Formulations

Embodiments of the invention include sterile pharmaceutical formulations of anti-CD105 antibodies that are useful as treatments for diseases. Such formulations would inhibit the binding of a native CD105-specific ligand such as, for example, TGF-β, to CD105, thereby effectively treating pathological conditions where, for example, serum or tissue CD105 expression is abnormally elevated. Anti-CD105 antibodies preferably possess adequate affinity to potently inhibit native CD105-specific ligands such as, for example, TGF-β, and preferably have an adequate duration of action to allow for infrequent dosing in humans. A prolonged duration of action will allow for less frequent and more convenient dosing schedules by alternate parenteral routes such as subcutaneous or intramuscular injection.

Sterile formulations can be created, for example, by filtration through sterile filtration membranes, prior to or following lyophilization and reconstitution of the antibody. The antibody ordinarily will be stored in lyophilized form or in solution. Therapeutic antibody compositions generally are placed into a container having a sterile access port, for example, an intravenous solution bag or vial having an adapter that allows retrieval of the formulation, such as a stopper pierceable by a hypodermic injection needle.

The route of antibody administration is in accord with known methods, e.g., injection or infusion by intravenous, intraperitoneal, intracerebral, intramuscular, intraocular, intraarterial, intrathecal, inhalation or intralesional routes, direct injection to a tumour site, or by sustained release systems as noted below. The antibody is preferably administered continuously by infusion or by bolus injection.

An effective amount of antibody to be employed therapeutically will depend, for example, upon the therapeutic objectives, the route of administration, and the condition of the patient. Accordingly, it is preferred that the therapist titer the dosage and modify the route of administration as required to obtain the optimal therapeutic effect. Typically, the clinician will administer antibody until a dosage is reached that achieves the desired effect. The progress of this therapy is easily monitored by conventional assays or by the assays described herein.

Antibodies, as described herein, can be prepared in a mixture with a pharmaceutically acceptable carrier. This therapeutic composition can be administered intravenously or through the nose or lung, preferably as a liquid or powder aerosol (lyophilized). The composition may also be administered parenterally or subcutaneously as desired. When administered systemically, the therapeutic composition should be sterile, pyrogen-free and in a parenterally acceptable solution having due regard for pH, isotonicity, and stability. These conditions are known to those skilled in the art. Briefly, dosage formulations of the compounds described herein are prepared for storage or administration by mixing the compound having the desired degree of purity with pharmaceutically acceptable carriers, excipients, or stabilizers. Such materials are non-toxic to the recipients at the dosages and concentrations employed, and include buffers such as TRIS HCl, phosphate, citrate, acetate and other organic acid salts; antioxidants such as ascorbic acid; low molecular weight (less than about ten residues) peptides such as polyarginine, proteins, such as serum albumin, gelatin, or immunoglobulins; hydrophilic polymers such as polyvinylpyrrolidinone; amino acids such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium and/or nonionic surfactants such as TWEEN, PLURONICS or polyethyleneglycol.

Sterile compositions for injection can be formulated according to conventional pharmaceutical practice as described in Remington: The Science and Practice of Pharmacy (20^{th} ed, Lippincott Williams & Wilkens Publishers (2003)). For example, dissolution or suspension of the active compound in a pharmaceutically acceptable carrier such as water or naturally occurring vegetable oil like sesame, peanut, or cottonseed oil or a synthetic fatty vehicle like ethyl oleate or the like may be desired. Buffers, preservatives, antioxidants and the like can be incorporated according to accepted pharmaceutical practice.

Suitable examples of sustained-release preparations include semipermeable matrices of solid hydrophobic polymers containing the polypeptide, which matrices are in the form of shaped articles, films or microcapsules. Examples of sustained-release matrices include polyesters, hydrogels (e.g., poly(2-hydroxyethyl-methacrylate) as described by Langer et al., J. Biomed Mater. Res., (1981) 15:167-277 and Langer, Chem. Tech., (1982) 12:98-105, or poly(vinylalcohol)), polylactides (U.S. Pat. No. 3,773,919, EP 58,481), copolymers of L-glutamic acid and gamma ethyl-L-glutamate (Sidman et al., Biopolymers, (1983) 22:547-556), non-degradable ethylene-vinyl acetate (Langer et al., supra), degradable lactic acid-glycolic acid copolymers such as the LUPRON Depot™ (injectable microspheres composed of lactic acid-glycolic acid copolymer and leuprolide acetate), and poly-D-(−)-3-hydroxybutyric acid (EP 133,988).

While polymers such as ethylene-vinyl acetate and lactic acid-glycolic acid enable release of molecules for over 100 days, certain hydrogels release proteins for shorter time periods. When encapsulated proteins remain in the body for a long time, they may denature or aggregate as a result of exposure to moisture at 37° C., resulting in a loss of biological activity and possible changes in immunogenicity. Rational strategies can be devised for protein stabilization depending on the mechanism involved. For example, if the aggregation mechanism is discovered to be intermolecular S—S bond formation through disulfide interchange, stabilization may be achieved by modifying sulfhydryl residues, lyophilizing from acidic solutions, controlling moisture content, using appropriate additives, and developing specific polymer matrix compositions.

Sustained-released compositions also include preparations of crystals of the antibody suspended in suitable formulations capable of maintaining crystals in suspension. These preparations when injected subcutaneously or intraperitonealy can produce a sustained release effect. Other compositions also include liposomally entrapped antibodies. Liposomes containing such antibodies are prepared by methods known per se: U.S. Pat. No. DE 3,218,121; Epstein et al., Proc. Natl. Acad. Sci. USA, (1985) 82:3688-3692; Hwang et al., Proc. Natl. Acad. Sci. USA, (1980) 77:4030-4034; EP 52,322; EP 36,676; EP 88,046; EP 143,949; 142,641; Japanese patent application 83-118008; U.S. Pat. Nos. 4,485,045 and 4,544,545; and EP 102,324.

The dosage of the antibody formulation for a given patient will be determined by the attending physician taking into consideration various factors known to modify the action of drugs including severity and type of disease, body weight, sex, diet, time and route of administration, other medications and other relevant clinical factors. Therapeutically effective dosages may be determined by either in vitro or in vivo methods.

An effective amount of the antibodies, described herein, to be employed therapeutically will depend, for example, upon the therapeutic objectives, the route of administration, and the condition of the patient. Accordingly, it is preferred for the therapist to titer the dosage and modify the route of administration as required to obtain the optimal therapeutic effect. A typical daily dosage might range from about 0.0001 mg/kg, 0.001 mg/kg, 0.01 mg/kg, 0.1 mg/kg, 1 mg/kg, 10 mg/kg to up to 100 mg/kg, 1000 mg/kg, 10000 mg/kg or more, of the patient's body weight depending on the factors mentioned above. The dosage may be between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight depending on the factors mentioned above. Typically, the clinician will administer the therapeutic antibody until a dosage is reached that achieves the desired effect. The progress of this therapy is easily monitored by conventional assays or as described herein.

Doses of antibodies of the invention may be repeated and the administrations may be separated by at least 1 day, 2 days, 3 days, 5 days, 10 days, 15 days, 30 days, 45 days, 2 months, 75 days, 3 months, or at least 6 months.

It will be appreciated that administration of therapeutic entities in accordance with the compositions and methods herein will be administered with suitable carriers, excipients, and other agents that are incorporated into formulations to provide improved transfer, delivery, tolerance, and the like. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol. Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

### Design and Generation of Other Therapeutics

In accordance with the present invention and based on the activity of the antibodies that are produced and characterized herein with respect to CD105, the design of other therapeutic modalities beyond antibody moieties is facilitated. Such modalities include, without limitation, advanced antibody therapeutics, such as bispecific antibodies, immunotoxins, and radiolabeled therapeutics, single domain antibodies, antibody fragments, such as a Fab, Fab′, F(ab′)₂, Fv or dAb, generation of peptide therapeutics, CD105 binding domains in novel scaffolds, gene therapies, particularly intrabodies, antisense therapeutics, and small molecules.

An antigen binding site may be provided by means of arrangement of CDRs on non-antibody protein scaffolds, such as fibronectin or cytochrome B etc. (Haan & Maggos (2004) BioCentury, 12(5): A1-A6; Koide et al. (1998) Journal of Molecular Biology, 284: 1141-1151; Nygren et al. (1997) Current Opinion in Structural Biology, 7: 463-469) or by randomising or mutating amino acid residues of a loop within a protein scaffold to confer binding specificity for a desired target. Scaffolds for engineering novel binding sites in proteins have been reviewed in detail by Nygren et al. (Nygren et al. (1997) Current Opinion in Structural Biology, 7: 463-469). Protein scaffolds for antibody mimics are disclosed in WO/0034784, which is herein incorporated by reference in its entirety, in which the inventors describe proteins (antibody mimics) that include a fibronectin type III domain having at least one randomised loop. A suitable scaffold into which to graft one or more CDRs, e.g. a set of HCDRs, may be provided by any domain member of the immunoglobulin gene superfamily. The scaffold may be a human or non-human protein. An advantage of a non-antibody protein scaffold is that it may provide an antigen-binding site in a scaffold molecule that is smaller and/or easier to manufacture than at least some antibody molecules. Small size of a binding member may confer useful physiological properties, such as an ability to enter cells, penetrate deep into tissues or reach targets within other structures, or to bind within protein cavities of the target antigen. Use of antigen binding sites in non-antibody protein scaffolds is reviewed in Wess, 2004 (Wess, L. In: BioCentury, The Bernstein Report on BioBusiness, 12(42), A1-A7, 2004). Typical are proteins having a stable backbone and one or more variable loops, in which the amino acid sequence of the loop or loops is specifically or randomly mutated to create an antigen-binding site that binds the target antigen. Such proteins include the IgG-binding domains of protein A from S. aureus, transferrin, albumin, tetranectin, fibronectin (e.g. 10th fibronectin type III domain), lipocalins as well as gamma-crystalline and other Affilin™ scaffolds (Scil Proteins). Examples of other approaches include synthetic “Microbodies” based on cyclotides—small proteins having intra-molecular disulphide bonds, Microproteins (Versabodies™, Amunix) and ankyrin repeat proteins (DARPins, Molecular Partners).

In addition to antibody sequences and/or an antigen-binding site, a targeted binding agent according to the present invention may comprise other amino acids, e.g. forming a peptide or polypeptide, such as a folded domain, or to impart to the molecule another functional characteristic in addition to ability to bind antigen. Targeted binding agents of the invention may carry a detectable label, or may be conjugated to a toxin or a targeting moiety or enzyme (e.g. via a peptidyl bond or linker). For example, a targeted binding agent may comprise a catalytic site (e.g. in an enzyme domain) as well as an antigen binding site, wherein the antigen binding site binds to the antigen and thus targets the catalytic site to the antigen. The catalytic site may inhibit biological function of the antigen, e.g. by cleavage.

In connection with the generation of advanced antibody therapeutics, where complement fixation is a desirable attribute, it may be possible to sidestep the dependence on complement for cell killing through the use of bispecific antibodies, immunotoxins, or radiolabels, for example.

For example, bispecific antibodies can be generated that comprise (i) two antibodies one with a specificity to CD105 and another to a second molecule that are conjugated together, (ii) a single antibody that has one chain specific to CD105 and a second chain specific to a second molecule, or (iii) a single chain antibody that has specificity to CD105 and the other molecule. Such bispecific antibodies can be generated using techniques that are well known; for example, in connection with (i) and (ii) see e.g., Fanger et al. Immunol Methods 4:72-81 (1994) and Wright and Harris, supra. and in connection with (iii) see e.g., Traunecker et al. Int. J. Cancer (Suppl.) 7:51-52 (1992). In each case, the second specificity can be made to the heavy chain activation receptors, including, without limitation, CD16 or CD64 (see e.g., Deo et al. Immunol. Today 18:127 (1997)) or CD89 (see e.g., Valerius et al. Blood 90:4485-4492 (1997)).

Antibodies can also be modified to act as immunotoxins, utilizing techniques that are well known in the art. See e.g., Vitetta Immunol Today 14:252 (1993). See also U.S. Pat. No. 5,194,594. In connection with the preparation of radiolabeled antibodies, such modified antibodies can also be readily prepared utilizing techniques that are well known in the art. See e.g., Junghans et al. in Cancer Chemotherapy and Biotherapy 655-686 (2d edition, Chafner and Longo, eds., Lippincott Raven (1996)). See also U.S. Pat. Nos. 4,681,581, 4,735,210, 5,101,827, 5,102,990 (RE 35,500), 5,648,471, and 5,697,902. Each immunotoxin or radiolabeled molecule would be likely to kill cells expressing the desired multimeric enzyme subunit oligomerisation domain.

When an antibody is linked to an agent (e.g., radioisotope, pharmaceutical composition, or a toxin), it is contemplated that the agent possess a pharmaceutical property selected from the group of antimitotic, alkylating, antimetabolite, antiangiogenic, apoptotic, alkaloid, COX-2, and antibiotic agents and combinations thereof. The drug can be selected from the group of nitrogen mustards, ethylenimine derivatives, alkyl sulfonates, nitrosoureas, triazenes, folic acid analogs, anthracyclines, taxanes, COX-2 inhibitors, pyrimidine analogs, purine analogs, antimetabolites, antibiotics, enzymes, epipodophyllotoxins, platinum coordination complexes, vinca alkaloids, substituted ureas, methyl hydrazine derivatives, adrenocortical suppressants, antagonists, endostatin, taxols, camptothecins, oxaliplatin, doxorubicins and their analogs, and a combination thereof.

In one particular example, the targeting agents of the invention are conjugated to a therapeutic agent or toxin, e.g., members of the enediyne family of molecules, such as calicheamicin and esperamicin. Chemical toxins can also be taken from the group consisting of duocarmycin (U.S. Pat. Nos. 5,703,080; 4,923,990), methotrexate, doxorubicin, melphalan, chlorambucil, ARA-C, vindesine, mitomycin C, cis-platinum, etoposide, bleomycin and 5-fluorouracil. Examples of chemotherapeutic agents also include Adriamycin, Doxorubicin, 5-Fluorouracil, Cytosine arabinoside (Ara-C), Cyclophosphamide, Thiotepa, Taxotere (docetaxel), Busulfan, Cytoxin, Taxol, Methotrexate, Cisplatin, Melphalan, Vinblastine, Bleomycin, Etoposide, Ifosfamide, Mitomycin C, Mitoxantrone, Vincreistine, Vinorelbine, Carboplatin, Teniposide, Daunomycin, Caminomycin, Aminopterin, Dactinomycin, Mitomycins, Esperamicins (U.S. Pat. No. 4,675,187), Melphalan, and other related nitrogen mustards.

In certain embodiments, the targeting agents of the invention are conjugated to a cytostatic, cytotoxic or immunosuppressive agent. In one embodiment the cytotoxic agent is selected from the group consisting of an enediyne, a lexitropsin, a duocarmycin, a taxane, a cryptophysin, a baccatin derivative, a podophyllotoxin, a puromycin, a dolastatin, a maytansinoid, a dolastatin and a vinca alkaloid. In specific embodiments, the cytotoxic agent is paclitaxel, docetaxel, CC-1065, trichothene, SN-38, topotecan, morpholino-doxorubicin, rhizoxin, cyanomorpholino-doxorubicin, dolastatin-10, echinomycin, combretastatin, calicheamicin, vincristine, vinblastine, vindesine, vinorelbine, VP-16, camptothecin, epithilone A, epithilone B, nocodazole, coichicine, colcimid, estramustine, cemadotin, discodermolide, eleutherobin, maytansine DM-1, auristatin E, AEB, AEVB, AEFP, MMAE or netropsin (US publication No. 2005/0238649) and their derivatives thereof.

In certain other embodiments, the cytoxic agent is Maytansine or Maytansinoids, and derivatives thereof, wherein the targeting agents of the invention are conjugated to one or more maytansinoid molecules. Maytansinoids are mitototic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4364,866; 4,424,219; 4,450,254; 4,362,663; and 4,371,533. In an attempt to improve their therapeutic index, maytansine and maytansinoids have been conjugated to antibodies specifically binding to tumor cell antigens. Immunoconjugates containing maytansinoids and their therapeutic use are disclosed, for example, in U.S. Pat. Nos. 5,208,020, 5,416,064 and European Patent EP 0 425 235 B1. Liu et al., Proc. Natl. Acad. Sci. USA 93:8618-8623 (1996) described immunoconjugates comprising a maytansinoid designated DM1 linked to the monoclonal antibody C242 directed against human colorectal cancer. The conjugate was found to be highly cytotoxic towards cultured colon cancer cells, and showed antitumor activity in an in vivo tumor growth assay. Chari et al. Cancer Research 52:127-131 (1992) describe immunoconjugates in which a maytansinoid was conjugated via a disulfide linker to the murine antibody A7 binding to an antigen on human colon cancer cell lines, or to another murine monoclonal antibody TA.1 that binds the HER-2/neu oncogene. The cytotoxicity of the TA.1-maytansonoid conjugate was tested in vitro on the human breast cancer cell line SK-BR-3, which expresses 3×10⁵ HER-2 surface antigens per cell. The drug conjugate achieved a degree of cytotoxicity similar to the free maytansonid drug, which could be increased by increasing the number of maytansinoid molecules per antibody molecule. The A7-maytansinoid conjugate showed low systemic cytotoxicity in mice. Thus, the present invention contemplates targeting agents conjugated to maytansinoid agents for therapeutic treatment of certain cancers.

In certain other embodiments, another immunoconjugate of interest comprises an targeting agents of the invention are conjugated to one or more calicheamicin molecules. The calicheamicin family of antibiotics are capable of producing double-stranded DNA breaks at sub-picomolar concentrations. For the preparation of conjugates of the calicheamicin family, see U.S. Pat. Nos. 5,712,374, 5,714,586, 5,739,116, 5,367,285, 5,770,701, 5,770.710, 5,773,001, 5,877,296 (all to American Cyanamid Company). Structural analogues of calicheamicin which may be used include, but are not limited to, γ₁^{I}, γ₂^{I}, γ₃^{I}, N-acetyl-γ₁^{I}, PSAG and θ₁^{I} (Hinman et al. Cancer Research 53: 3336-3342 (1993), Lode et al. Cancer Research 58: 2925-2928 (1998) and the aforementioned U.S. patents to American Cyanamid). Another anti-tumor drug that the antibody can be conjugated is QFA which is an antifolate. Both calicheamicin and QFA have intracellular sites of action and do not readily cross the plasma membrane. Therefore, cellular uptake of these agents through antibody mediated internalization greatly enhances their cytotoxic effects.

Other toxins that can be used in immunoconjugates of the invention include poisonous lectins, plant toxins such as ricin, abrin, modeccin, botulina, and diphtheria toxins. Of course, combinations of the various toxins could also be coupled to one antibody molecule thereby accommodating variable cytotoxicity. Illustrative of toxins which are suitably employed in combination therapies of the invention are ricin, abrin, ribonuclease, DNase I, Staphylococcal enterotoxin-A, pokeweed anti-viral protein, gelonin, diphtherin toxin, Pseudomonas exotoxin, and Pseudomonas endotoxin. See, for example, Pastan et al., Cell, 47:641 (1986), and Goldenberg et al., Cancer Journal for Clinicians, 44:43 (1994). Enzymatically active toxins and fragments thereof which can be used include diphtheria A chain, non-binding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes.

Suitable toxins and chemotherapeutic agents are described in Remington's Pharmaceutical Sciences, 19th Ed. (Mack Publishing Co. 1995), and in Goodman And Gilman's The Pharmacological Basis of Therapeutics, 7th Ed. (MacMillan Publishing Co. 1985). Other suitable toxins and/or chemotherapeutic agents are known to those of skill in the art.

Examples of radioisotopes include gamma-emitters, positron-emitters, and x-ray emitters that can be used for localisation and/or therapy, and beta-emitters and alpha-emitters that can be used for therapy. The radioisotopes described previously as useful for diagnostics, prognostics and staging are also useful for therapeutics.

Non-limiting examples of anti-cancer or anti-leukemia agents include anthracyclines such as doxorubicin (adriamycin), daunorubicin (daunomycin), idarubicin, detorubicin, caminomycin, epirubicin, esorubicin, and morpholino and substituted derivatives, combinations and modifications thereof. Exemplary pharmaceutical agents include cis-platinum, taxol, calicheamicin, vincristine, cytarabine (Ara-C), cyclophosphamide, prednisone, daunorubicin, idarubicin, fludarabine, chlorambucil, interferon alpha, hydroxyurea, temozolomide, thalidomide, and bleomycin, and derivatives, combinations and modifications thereof. Preferably, the anti-cancer or anti-leukemia is doxorubicin, morpholinodoxorubicin, or morpholinodaunorubicin.

The antibodies of the invention also encompass antibodies that have half-lives (e.g., serum half-lives) in a mammal, preferably a human, of greater than that of an unmodified antibody. Said antibody half life may be greater than about 15 days, greater than about 20 days, greater than about 25 days, greater than about 30 days, greater than about 35 days, greater than about 40 days, greater than about 45 days, greater than about 2 months, greater than about 3 months, greater than about 4 months, or greater than about 5 months. The increased half-lives of the antibodies of the present invention or fragments thereof in a mammal, preferably a human, result in a higher serum titer of said antibodies or antibody fragments in the mammal, and thus, reduce the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor (see, e.g., International Publication Nos. WO 97/34631 and WO 02/060919, which are incorporated herein by reference in their entireties). Antibodies or fragments thereof with increased in vivo half-lives can be generated by attaching to said antibodies or antibody fragments polymer molecules such as high molecular weight polyethyleneglycol (PEG). PEG can be attached to said antibodies or antibody fragments with or without a multifunctional linker either through site-specific conjugation of the PEG to the N- or C-terminus of said antibodies or antibody fragments or via epsilon-amino groups present on lysine residues. Linear or branched polymer derivatisation that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography.

As will be appreciated by one of skill in the art, in the above embodiments, while affinity values can be important, other factors can be as important or more so, depending upon the particular function of the antibody. For example, for an immunotoxin (toxin associated with an antibody), the act of binding of the antibody to the target can be useful; however, in some embodiments, it is the internalisation of the toxin into the cell that is the desired end result. As such, antibodies with a high percent internalisation can be desirable in these situations. Thus, in one embodiment, antibodies with a high efficiency in internalisation are contemplated. A high efficiency of internalisation can be measured as a percent internalised antibody, and can be from a low value to 100%. For example, in varying embodiments, 0.1-5, 5-10, 10-20, 20-30, 30-40, 40-45, 45-50, 50-60, 60-70, 70-80, 80-90, 90-99, and 99-100% can be a high efficiency. As will be appreciated by one of skill in the art, the desirable efficiency can be different in different embodiments, depending upon, for example, the associated agent, the amount of antibody that can be administered to an area, the side effects of the antibody-agent complex, the type (e.g., cancer type) and severity of the problem to be treated.

In other embodiments, the antibodies disclosed herein provide an assay kit for the detection of CD105 expression in mammalian tissues or cells in order to screen for a disease or disorder associated with changes in expression of CD105. The kit comprises an antibody that binds CD105 and means for indicating the reaction of the antibody with the antigen, if present.

### Combinations

The targeted binding agent or antibody defined herein may be applied as a sole therapy or may involve, in addition to the compounds of the invention, conventional surgery or radiotherapy or chemotherapy. Such chemotherapy may include one or more of the following categories of anti tumour agents:

(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumor antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);

(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;

(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), and metalloproteinase inhibitors like marimastat, inhibitors of urokinase plasminogen activator receptor function or, inhibitors of cathepsins, inhibitors of serine proteases for example matriptase, hepsin, urokinase, inhibitors of heparanase);

(iv) cytotoxic agents such as fludarabine, 2-chlorodeoxyadenosine, chlorambucil or doxorubicin and combination thereoff such as Fludarabine+cyclophosphamide, CVP: cyclophosphamide+vincristine+prednisone, ACVBP: doxorubicin+cyclophosphamide+vindesine+bleomycin+prednisone, CHOP: cyclophosphamide+doxorubicin+vincristine+prednisone, CNOP: cyclophosphamide+mitoxantrone+vincristine+prednisone, m-BACOD: methotrexate+bleomycin+doxorubicin+cyclophosphamide+vincristine+dexamethasone+leucovorin., MACOP-B: methotrexate+doxorubicin+cyclophosphamide+vincristine+prednisone fixed dose+bleomycin+leucovorin, or ProMACE CytaBOM: prednisone+doxorubicin+cyclophosphamide+etoposide+cytarabine+bleomycin+vincristine+methotrexate+leucovorin.

(v) inhibitors of growth factor function, for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI-1033), erbB2 tyrosine kinase inhibitors such as lapatinib, inhibitors of the hepatocyte growth factor family, inhibitors of the platelet-derived growth factor family such as imatinib, inhibitors of serine/threonine kinases (for example Ras/Raf signalling inhibitors such as farnesyl transferase inhibitors, for example sorafenib (BAY 43-9006)), inhibitors of cell signalling through MEK and/or AKT kinases, inhibitors of the hepatocyte growth factor family, c-kit inhibitors, abl kinase inhibitors, IGF receptor (insulin-like growth factor) kinase inhibitors, aurora kinase inhibitors (for example AZD1152, PH739358, VX-680, MLN8054, R763, MP235, MP529, VX-528 and AX39459), cyclin dependent kinase inhibitors such as CDK2 and/or CDK4 inhibitors, and inhibitors of survival signaling proteins such as Bcl-2, Bcl-XL for example ABT-737;

(vi) antiangiogenic agents such as those which inhibit the effects of vascular endothelial growth factor, [for example the anti-vascular endothelial cell growth factor antibody bevacizumab (Avastin™), Sunitinib malate (Sutent™), Sorafenib (Nexavar™) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), compounds such as those disclosed in International Patent Applications WO97/22596, WO 97/30035, WO 97/32856, WO 98/13354, WO00/47212 and WO01/32651 and compounds that work by other mechanisms (for example linomide, inhibitors of integrin αvβ3 function and angiostatin)] or colony stimulating factor 1 (CSF1) or CSF1 receptor;

(vii) vascular damaging agents such as Combretastatin A4 and compounds disclosed in International Patent Applications WO 99/02166, WO 00/40529, WO 00/41669, WO 01/92224, WO 02/04434 and WO 02/08213;

(viii) antisense therapies, for example those which are directed to the targets listed above, such as G-3139 (Genasense), an anti bcl2 antisense;

(ix) gene therapy approaches, including for example approaches to replace aberrant genes such as aberrant p53 or aberrant BRCA1 or BRCA2, GDEPT (gene directed enzyme pro drug therapy) approaches such as those using cytosine deaminase, thymidine kinase or a bacterial nitroreductase enzyme and approaches to increase patient tolerance to chemotherapy or radiotherapy such as multi drug resistance gene therapy; and

(x) immunotherapy approaches, including for example treatment with Alemtuzumab (campath-1H™), a monoclonal antibody directed at CD52, or treatment with antibodies directed at CD22, ex vivo and in vivo approaches to increase the immunogenicity of patient tumour cells, transfection with cytokines such as interleukin 2, interleukin 4 or granulocyte macrophage colony stimulating factor, approaches to decrease T cell anergy such as treatment with monoclonal antibodies inhibiting CTLA-4 function, approaches using transfected immune cells such as cytokine transfected dendritic cells, approaches using cytokine transfected tumour cell lines and approaches using anti idiotypic antibodies.

(xi) inhibitors of protein degradation such as proteasome inhibitor such as Velcade (bortezomid).

(xii) biotherapeutic therapeutic approaches for example those which use peptides or proteins (such as antibodies or soluble external receptor domain constructions) which either sequester receptor ligands, block ligand binding to receptor or decrease receptor signalling (e.g. due to enhanced receptor degradation or lowered expression levels).

In one embodiment the anti-tumour treatment defined herein may involve, in addition to the compounds of the invention, treatment with other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin).

In one embodiment the anti-tumour treatment defined herein may involve, in addition to the compounds of the invention, treatment with gemcitabine.

Such conjoint treatment may be achieved by way of the simultaneous, sequential or separate dosing of the individual components of the treatment. Such combination products employ the compounds of this invention, or pharmaceutically acceptable salts thereof, within the dosage range described hereinbefore and the other pharmaceutically active agent within its approved dosage range.

### EXAMPLES

The following examples, including the experiments conducted and results achieved are provided for illustrative purposes only and are not to be construed as limiting upon the teachings herein.

### Example 1

### Immunization and Titering

### Cells and Transfection

The mouse pre-B cell line B300-19 was cultured in RPMI 1640 medium containing 10% fetal bovine serum, 50 μM 2-mercaptethanol, 100 U/ml penicillin, and 100 μg/ml streptomycin. HEK 293F cells were grown in DMEM/F12 (50/50 mix) media supplemented with 10% FBS, 2 mM L-Glutamine, 50 μM BME, 100 units Penicillin-g/ml, 100 units MCG Streptomycin/ml. A human CD105 expression plasmid was transfected into HEK 293F or B300.19 cells using LipofectAMINE 2000 Reagent (Invitrogen, Carlsbad, Calif.), according to the manufacturer's instructions. Transfection proceeded for 48 hours followed by selection with 1 mg/ml G418 (Invitrogen, Carlsbad, Calif.) for two weeks. Stable G418 resistant clones were stained with a primary mouse anti-human CD105 monoclonal antibody and analyzed by FACS. The B300.19 stable transfectants were used for immunization while the HEK293F stable tranfectants were used for screening.

### Immunization

Immunizations were conducted using recombinant soluble CD105 (R&D Systems, Catalog Number: 1097-EN-025/CF), or stably transfected B300.19 cells expressing human CD105.

For immunization with recombinant soluble CD105, 10 μg/mouse of soluble protein was provided in the initial boost, followed by 5 μg/mouse in subsequent boosts, using XenoMouse™ strains XM3B3L3:IgG1KL and XM3C1L3:IgG4KL. For immunizations using B300.19 transfectant cells stably expressing human CD105, monoclonal antibodies were developed by sequentially immunizing XenoMouse™ mice strains XM3C1L3:IgG4KL and XMG2L3:IgG2KL. XenoMouse animals were immunized via footpad route for all injections by conventional means. The total volume of each injection was 50 μl per mouse, 25 μl per footpad.

The immunization was carried out according to the methods disclosed in U.S. patent application Ser. No. 08/759,620, filed Dec. 3, 1996 and International Patent Application Nos. WO 98/24893, published Jun. 11, 1998 and WO 00/76310, published Dec. 21, 2000, the disclosures of which are hereby incorporated by reference. The immunization programs are summarized in Table 3.

### Selection of Animals for Harvest by Titer

Titers of the antibody against human CD105 were tested by FACS staining for native antigen binding using Human Umbilical Vein Endothelial Cells (HUVEC). At the end of the immunization program, fusions were performed using mouse myeloma cells and lymphocytes isolated from the spleens and lymph nodes of the immunized mice by means of electroporation, as described in Example 2.

TABLE 3
Summary of Immunization Programs

| | | | | Number | |
|---|---|---|---|---|---|
| | | | | of | Immunization |
| Campaign | Group | Immunogen | Strain | mice | routes |
| 1 | 1 | Recombinant | IgG1 | 10 | Footpad, |
| | | soluble CD105 | | | twice/wk, x |
| | | (R&D Systems: | | | 4 weeks |
| | | Catalog#: | | | |
| | | 1097-EN- | | | |
| | | 025/CF) | | | |
| 1 | 1 | Recombinant | IgG4 | 10 | Footpad, |
| | | soluble CD105 | | | twice/wk, x |
| | | (R&D Systems: | | | 4 weeks |
| | | Catalog#: | | | |
| | | 1097-EN- | | | |
| | | 025/CF) | | | |
| 2 | 2 | B300.19/human | IgG2 | 10 | IP/Tail/BIP, |
| | | CD105 | | | twice/wk, |
| | | | | | x 8 wks, |
| | | | | | followed by |
| | | | | | IP/Tail/BIP, |
| | | | | | once/every |
| | | | | | 2 weeks, x |
| | | | | | 6 wks |
| 2 | 2 | B300.19/human | IgG4 | 10 | IP/Tail/BIP, |
| | | CD105 | | | twice/wk, |
| | | | | | x 8 wks, |
| | | | | | followed by |
| | | | | | IP/Tail/BIP, |
| | | | | | once/every |
| | | | | | 2 weeks, x |
| | | | | | 6 wks |

“IP” refers to “intraperitoneal”
“BIP” refers to “Base of Tail/Intraperitoneal”

### Example 2

### Recovery of Lymphocytes, B-Cell Isolations, Fusions and Generation Of Hybridomas

Immunized mice were sacrificed by cervical dislocation, and the draining lymph nodes harvested and pooled from each cohort. There were four harvests performed for this program.

The lymphoid cells were dissociated by grinding in DMEM to release the cells from the tissues and the cells were suspended in DMEM. The cells were counted, and 0.9 ml DMEM per 100 million lymphocytes added to the cell pellet to resuspend the cells gently but completely. Using 100 μl of CD90+ magnetic beads per 100 million cells, the cells were labeled by incubating the cells with the magnetic beads at 4° C. for 15 minutes. The magnetically labeled cell suspension containing up to 10⁸ positive cells (or up to 2×10⁹ total cells) was loaded onto a LS+column and the column washed with DMEM. The total effluent was collected as the CD90-negative fraction (most of these cells were expected to be B cells).

The fusion was performed by mixing washed enriched Day 6 B cells with nonsecretory myeloma P3X63Ag8.653 cells purchased from ATCC, cat.# CRL 1580 (Kearney et al, J. Immunol. 123, 1979, 1548-1550) at a ratio of 1:4. The cell mixture was gently pelleted by centrifugation at 400×g for 4 minutes. After decanting of the supernatant, the cells were gently mixed using a 1 ml pipette. Preheated PEG (1 ml per 10⁶ B-cells) was slowly added with gentle agitation over 1 minute followed by 1 minute of mixing. Preheated IDMEM (2 ml per 10⁶ B-cells) was then added over 2 minutes with gentle agitation. Finally preheated IDMEM (8 ml per 10⁶ B-cells) was added over 3 minutes.

The fused cells were spun down at 400×g for 6 minutes and resuspended in 20 ml of Selection media (DMEM (Invitrogen), 15% FBS (Hyclone), supplemented with L-glutamine, pen/strep, MEM Non-essential amino acids, Sodium Pyruvate, 2-Mercaptoethanol (all from Invitrogen), HA-Azaserine Hypoxanthine and OPI (oxaloacetate, pyruvate, bovine insulin) (both from Sigma) and IL-6 (Boehringer Mannheim)) per 10⁶ B-cells. Cells were incubated for 20-30 minutes at 37° C. and then resuspended in 200 ml Selection media and cultured for 3-4 days in a T175 flask.

On day 3 post fusion, the cells were collected, spun for 8 minutes at 400×g and resuspended in 10 ml Selection media per 10⁶ fused B-cells. FACS analysis of hybridoma population was performed, and cells were subsequently frozen down.

Hybridomas were grown as routine in the selective medium. Exhaustive supernatants collected from the hybridomas that potentially produce anti-human CD105 antibodies were subjected to subsequent screening assays.

### Example 3

### Selection of Candidate Antibodies by FMAT and FACS

After 14 days of culture, hybridoma supernatants were screened for CD105-specific antibodies by Fluorometric Microvolume Assay Technology (FMAT). Hybridoma supernatants were screened against HEK293F transfectant cells stably expressing human CD105 and counter-screened against parental HEK293F cells.

The culture supernatants from the CD105-positive hybridoma cells (based on the primary screen) were removed and the CD105 positive hybridoma cells were suspended with fresh hybridoma culture medium and transferred to 24-well plates. After two days in culture, these supernatants were evaluated in a secondary confirmation screen. In the secondary confirmation screen, the positives previously identified were screened by FMAT and/or FACS on HUVEC cells using two or three sets of detection antibodies used separately: 1.25 ug/ml GAH-Gamma Cy5 (JIR#109-176-098) for human gamma chain detection; 1.25 ug/ml GAH-Kappa PE (S.B.#2063-09) for human kappa light chain detection and 1.25 ug/ml GAH-lambda PE (S.B.#2073-09) for human lambda light chain detection in order to confirm that the anti-CD105 antibodies were fully human.

A total of 824 fully human anti-CD105 antibodies were identified from the first campaign as determined by FMAT using HEK293F transfectant cells stably expressing human CD105. For the second immunization campaign, a total of 788 fully human anti-CD105 antibodies were generated as determined by FMAT using HEK293F transfectant cells stably expressing human CD105. For both campaigns, antibodies were subsequently screened by FMAT and/or FACS on HUVEC cells and evaluated for cross-reactivity to cynomologus monkey and murine CD105 orthologs. CD105 derived from cynomolgus monkey and mouse were cloned and expressed on the surface of HEK293F cells for use in cross-reactivity studies. Antibodies exhibiting cross-reactivity to cynomologus monkey and mouse were carried forward and further evaluated in functional assays.

TABLE 4
Fully human CD105 specific monoclonal antibodies.

| | | | | FMAT/FACS | |
|---|---|---|---|---|---|
| | | FMAT | FMAT/FACS | Cynomologus | FMAT/FACS |
| Campaign | Antigen | (HEK293/huCD105) | (HUVEC cells) | Monkey | Mouse |
| 1 | Soluble CD105 | 824 | 621 | 140 | 9 |
| 2 | B300.19/huCD105 | 788 | 461 | 416 | 8 |

### Example 4

### Anti-Proliferative Activity

To screen and identify antibody lines exhibiting anti-proliferative activity in the HUVEC cell line, the Alamar Blue assay was performed. In brief, HUVEC cells were obtained from Cambrex Corp. and were maintained in EGM2 medium supplemented with 0.5% FBS. Cells were seeded at a concentration of 1000 cells/well (90 μl/well) in 96-well plates. Cells were incubated at 37° C. and 5% CO₂ for 72 hours. The assay was terminated 72 hours post addition of the antibodies and an Alamar Blue assay was conducted. Cells were treated with antibody at a concentration of 50 μg/ml. The determination of percent survival for the treatment samples was based on normalizing the control sample (i.e. no treatment) to 100% viable.

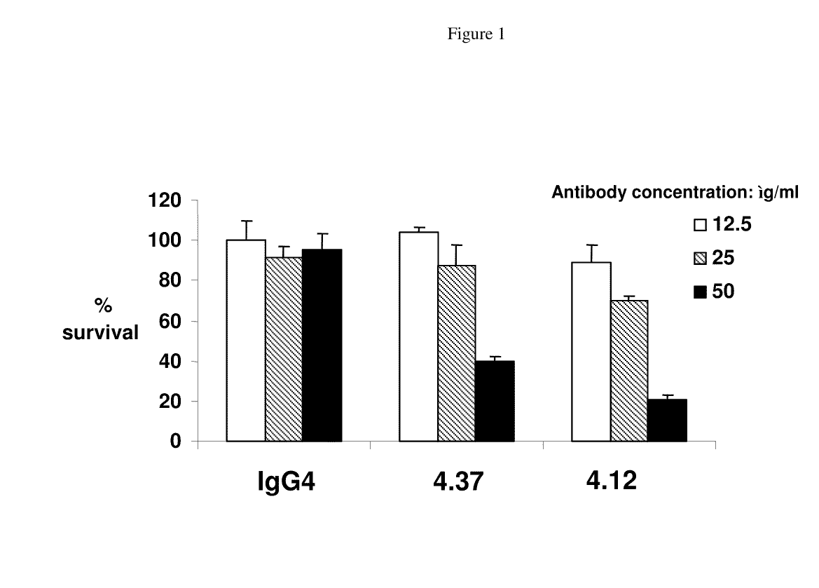

Analysis revealed that a majority of the anti-CD105 antibody hybridoma lines did not exhibit anti-proliferative activity. As shown in FIG. 1, from Campaign 1, two hybridomas were identified, designated 4.120 and 4.37, and exhibited pronounced inhibition of cell proliferation at an antibody concentration of 50 μg/ml.

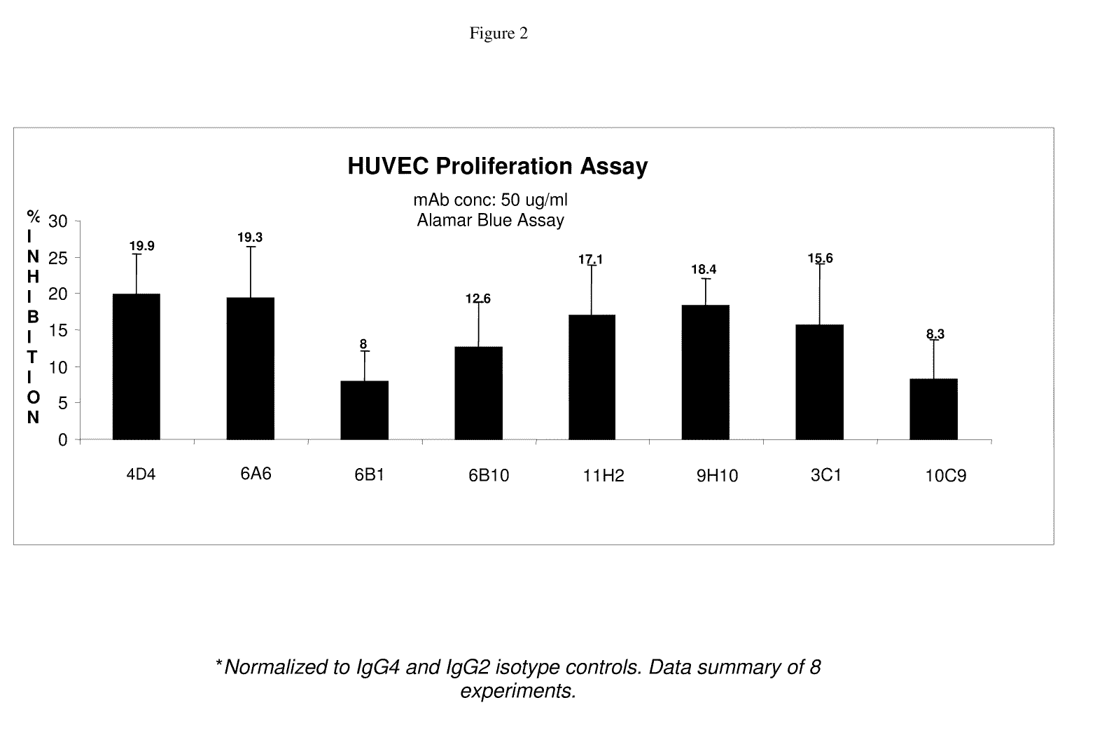

For campaign 2, eight additional lines were identified and interrogated in the proliferation assay. As shown in FIG. 2 and Table 5, inhibition of cell proliferation ranged on average from 8% to 20%.

TABLE 5

| | % Inhibition | % Inhibition | % Inhibition | % Inhibition | % Inhibition | % Inhibition | % Inhibition | % Inhibition | | |
|---|---|---|---|---|---|---|---|---|---|---|
| | Experiment | Experiment | Experiment | Experiment | Experiment | Experiment | Experiment | Experiment | % Inhibition | |
| mAb | 1 | 2 | 3 | 4 | 5 | 6 | 7 | 8 | Average | Std Dev. |
| 4D4.1 | 22.18 | 27 | 16 | 14.83 | 14.81 | 29.15 | 16.8 | 18.31 | 19.9 | 5.61 |
| 6A6.2 | 9.57 | 28.26 | 21.12 | 21.6 | 15.33 | 20.52 | 19.38 | 18.4 | 19.27 | 5.37 |
| 6B1.1 | 13.14 | −4.12 | 15.47 | 5.27 | 9.05 | 7.33 | 11.74 | 6.07 | 7.99 | 6.05 |
| 6B10.1 | 14.05 | 21.56 | 17.58 | 10.85 | 8.66 | 10.88 | 5.1 | 11.98 | 12.58 | 5.15 |
| 11H2.1 | 14.24 | 32.05 | 11.7 | 25.22 | 16.9 | 11.64 | 20.8 | 4.11 | 17.09 | 8.78 |
| 9H10.2 | 21.41 | 27.47 | 11.02 | 28.09 | 11.85 | 16.87 | 17.22 | 12.98 | 18.36 | 6.72 |
| 3C1.1 | 14.51 | 25.31 | 5.6 | 22.8 | 11.26 | 15.37 | 19.62 | 10.03 | 15.56 | 6.69 |
| 10C9.2 | 11.2 | 8.36 | 1.63 | 15.46 | 11.41 | 14.49 | 0.31 | 3.54 | 8.3 | 5.84 |

### Example 5

### Smad2 Phosphorylation Assay

In order to determine whether anti-CD105 antibodies mediate increased phosphorylation of Smad2, a Smad2 phosphorylation assay was performed. Briefly, 90,000 HUVEC cells were seeded per well in a 6-well plate. Cells were cultured in EGM2 medium supplemented with 0.5% FBS. Cells were treated with increasing concentrations of antibody (0.5, 1.0, and 2.0 μg/ml) for 24 hours followed by Western blot analysis. Detection of phospho-Smad2 was performed using a pSmad2 specific antibody (Cell Signaling Cat #3101). Total Smad2 levels were detected using a specific Smad2 antibody (Cell Signaling Cat #3102). The results show that antibody 4.37 mediated a dose-dependent inhibition of Smad2 phosphorylation. These findings were also confirmed for antibodies 4.120, 4D4, 6B10, 6A6, and 9H10. It is important to note that phosphorylation of Smad2 has been reported to inhibit endothelial cell proliferation and migration (Goumans M-J et al., EMBO J. 2002; 21:1743-1753).

### Example 6

### CD105 Inhibitory Antibody Reduces Tube Formation In Vitro

CD105 inhibitory antibodies were tested for the ability to reduce endothelial cell tube formation in an in vitro co-culture assay (TCS Cell Works Cat no. ZHA-1000). On day 1, Human Umbilical Vein Endothelial Cells (HUVECs) and human diploid fibroblasts were obtained as co-cultures in 24 well plates. CD105 blocking antibodies were introduced to the cultures on day 1 and at regular intervals over an 11-day period at an antibody concentration of 50 μg/mL. Media was replenished on days 4, 7 and 9. The co-culture model was maintained in either TCS Optimised medium (supplied with the co-culture assay) or in MCDB131 medium supplemented with 2% fetal calf serum (FCS), 1% glutamine and 1% penicillin/streptomycin (hereafter referred to as 2% FS MCDB131 medium). The co-culture model was maintained at 37° C. in a humidified 5% CO₂/95% air atmosphere.

Tubule formation was examined at day 11 following fixing and staining of tubules for CD31 using a tubule staining kit according to the manufacturors instructions (TCS Cell Works Cat no. ZHA-1225). Briefly, cells were fixed with ice-cold 70% ethanol for 30 minutes at room temperature (RT). Cells were blocked after which they were treated with anti-human CD31 for 60 minutes at RT. Plates were washed and treated with goat anti-mouse IgG conjugated with alkaline phosphatase (AP) for 60 minutes at RT. After incubation with the AP-conjugated secondary antibody, the plates were washed and 5-bromo-4-chloro-3-indolyl phosphate/nitro blue tetrazolium (BCIP/NBT) substrate was added for approximately 10 minutes. The development of a dark purple colour within 10 minutes reflected tubule formation. Plates were subsequently washed and left to air dry.

Quantification of tubule growth was conducted by whole-well image analysis methodology using a Zeiss KS400 3.0 Image Analyser. The morphological parameter measured in the quantification methodology was total tubule length. All tubule formations within each of the 24 wells were measured excluding a rim of 100 μm depth to avoid edge retraction artifact.

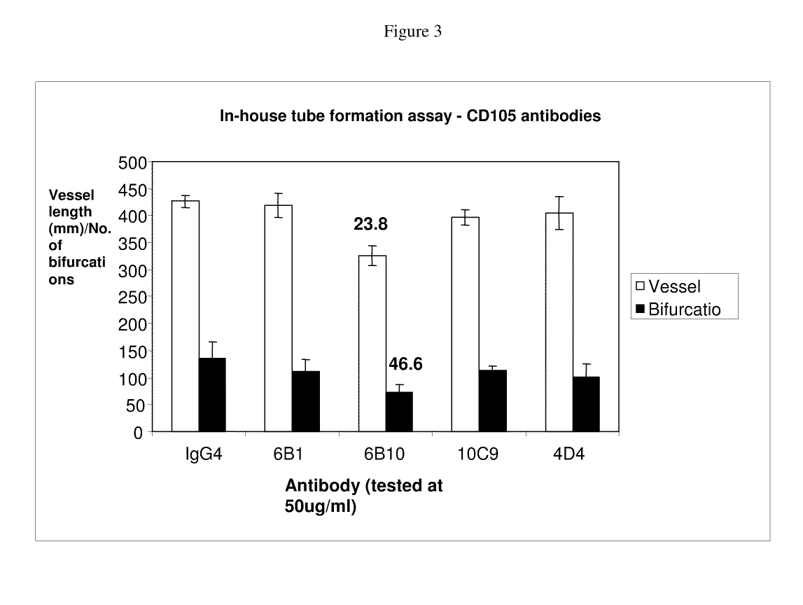

As illustrated in FIG. 3, mAb 6B10 was effective in inhibiting endothelial cell tube formation in vitro. This antibody inhibited vessel length by approximately 24% and inhibited the number of bifurcations by 47% relative to isotype control. The data indicates that this antibody is active in a functional assay that models the angiogenic process.

### Example 7

### Actin Modulation Assay

In order to ascertain whether the panel of anti-CD105 antibodies from Campaigns 1 and 2 impacts the cytoskeletal structure of human endothelial cells, an actin modulation assay was performed. Briefly, HUVEC cells were seeded into 4-well chamber slides (40,000 cells/well) and maintained in EGM2 medium supplemented with 0.5% FBS. Anti-CD105 antibodies were incubated with the HUVEC cells for 72 hours at an antibody concentration of 30 μg/ml. Following antibody incubation, cells were fixed with 4% formaldehyde for 10 minutes followed by permeabilization with 0.5% Triton X-100 for 10 minutes. Following permeabilization, cells were stained with Alexa Fluor 488 Phalloidin (Phalloidin, Molecular Probes, #A12379) for 30 minutes at room temperature. Cells were washed with PBS following staining and examined using a confocal microscope. Monoclonal antibodies 10C9, 3C1, 6B1, 4.120, 4.37, and 6B10 mediated pronounced modulation of the actin cytoskeletal structure in HUVEC cells.

### Example 8

### Epitope Binning of XenoMouse Monoclonal Anti-CD105 Antibodies as Compared to Antibody SN6 (HUVECS)

A FACS-based binning analysis was performed on human umbilical vein endothelial cells (HUVECs) to ascertain whether the panel of XenoMouse antibodies cross-compete with the commercially available SN6 antibody. The Seon laboratory first generated the SN6 antibody; this antibody is one of a panel of mAbs, denoted as the SN6 series, that is reported to suppress growth of human umbilical vein endothelial cells (HUVECs) in a dose-dependent manner (She X et al., Int. J. Cancer, 2004, 108: 251-7).

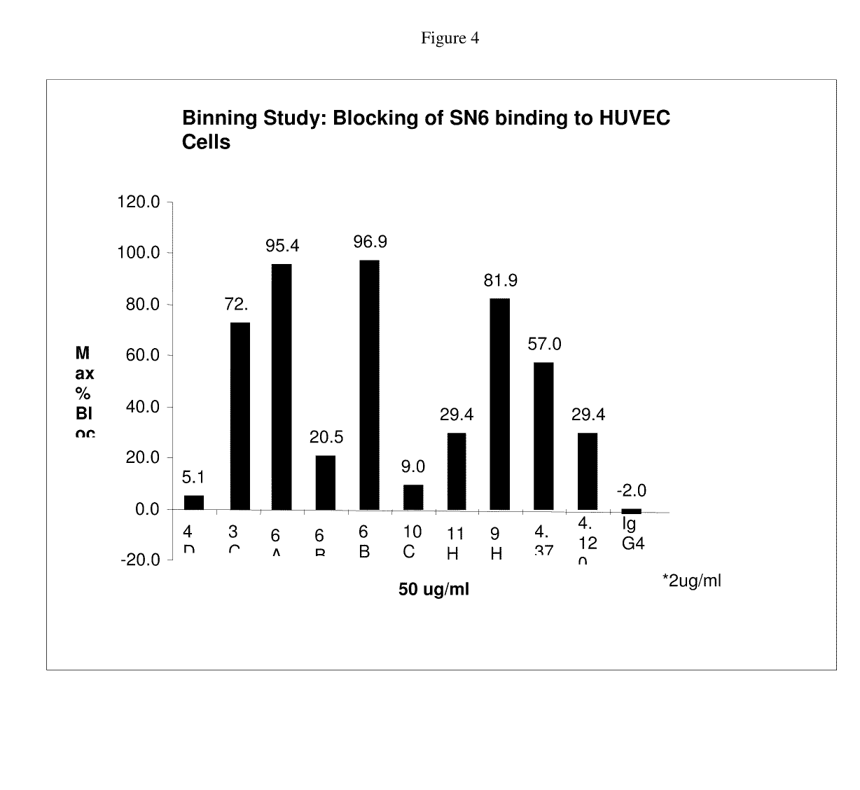

In brief, a titration on HUVEC cells was performed with the SN6 antibody (Abcam) fluorescently labeled with FITC. The EC₅₀ concentration for binding was determined to be 2 μg/ml. Subsequently, HUVEC cells were incubated with titrations of unlabeled XenoMouse anti-CD105 mAbs, washed, and then incubated with 2 μg/ml of the SN6 antibody. As shown in FIG. 4, the percent binding of the SN6 antibody is indicated in the presence of 50 μg/ml of the unlabeled XenoMouse mAbs.

Interestingly, antibodies 6A6 and 6B10 demonstrated pronounced inhibition of SN6 binding, suggesting these mAbs compete for the same epitope. Other antibodies partially competed with SN6, suggesting partial or overlapping epitopes. Antibodies 4D4 and 10C9 exhibited weak blocking, implying that these mAbs may not share the same epitope with the SN6 antibody. Equally important, these results suggest that this panel of XenoMouse anti-CD105 antibodies exhibits broad epitope specificity.

### Example 9

### Colo0205 Matrigel Plug Assay in CD105 KI/KO Mouse

In order to examine the in vivo activity of the XenoMouse mAbs, a matrigel plug assay was performed. Due to the lack of mouse cross-reactivity of the anti-CD105 antibodies, this study was performed in CD105 KI/KO-SCID animals.

In brief, five million Colo205 tumor cells mixed with matrigel were implanted into CD105 KI/KO-SCID mice. Mice received twice weekly treatment of antibody i.p. at an antibody dose of 10 mpk. Plugs were isolated on day 8 and analyzed for CD31 expression by IHC and hemoglobin content. IHC staining was performed using an anti-CD31 antibody (BD, Cat 550274). Samples were fixed in zinc fixative and embedded in paraffin blocks. Tissue sections were stained with CD31 antibody using Ventana automation. Images were scanned using the Aperio imaging system. IHC-positive staining was analyzed using the Aperio color deconvolution imaging software.

For hemoglobin content, deionized water, based on the plug's weight (5.0 ml/g), was added to the matrigel sample tube. The matrigel sample was then homogenized using a Polytron homogenizer. The sample was subsequently centrifuged at 3700 rpm for 10 min. A 250 uL aliquot of supernatant was mixed with an equal volume of 2× Drabkin's solution. The mixture was vortexed and centrifuged again. A 200 uL aliquot of this mixture was plated into a 96 well plate for analysis. Absorbance was measured at 540 nm. In parallel, a standard curve dilution was performed using a hemoglobin standard in 1× Drabkin's solution. The sample concentration was determined from the standard curve.

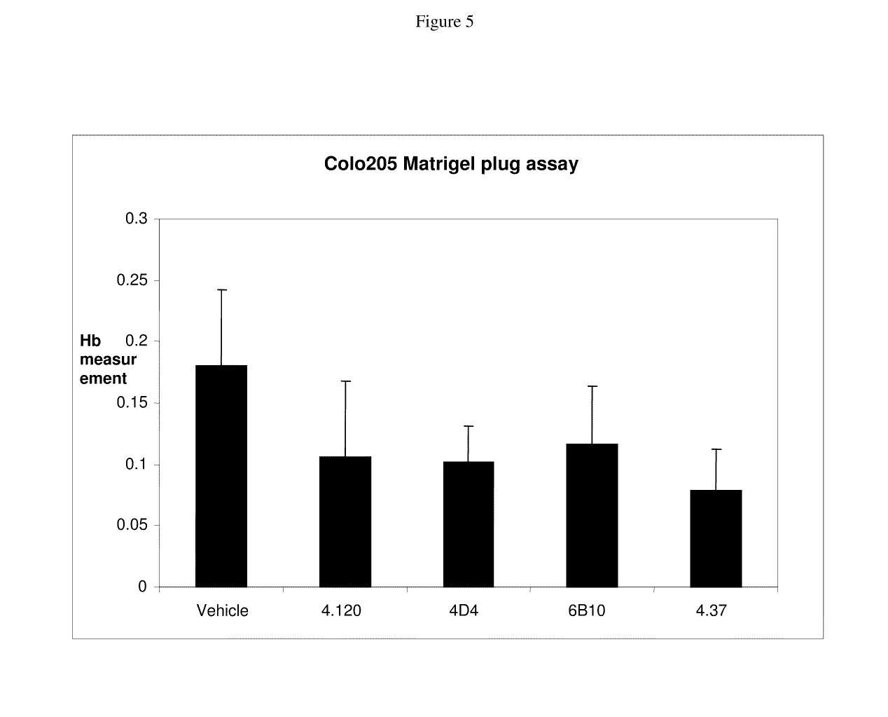

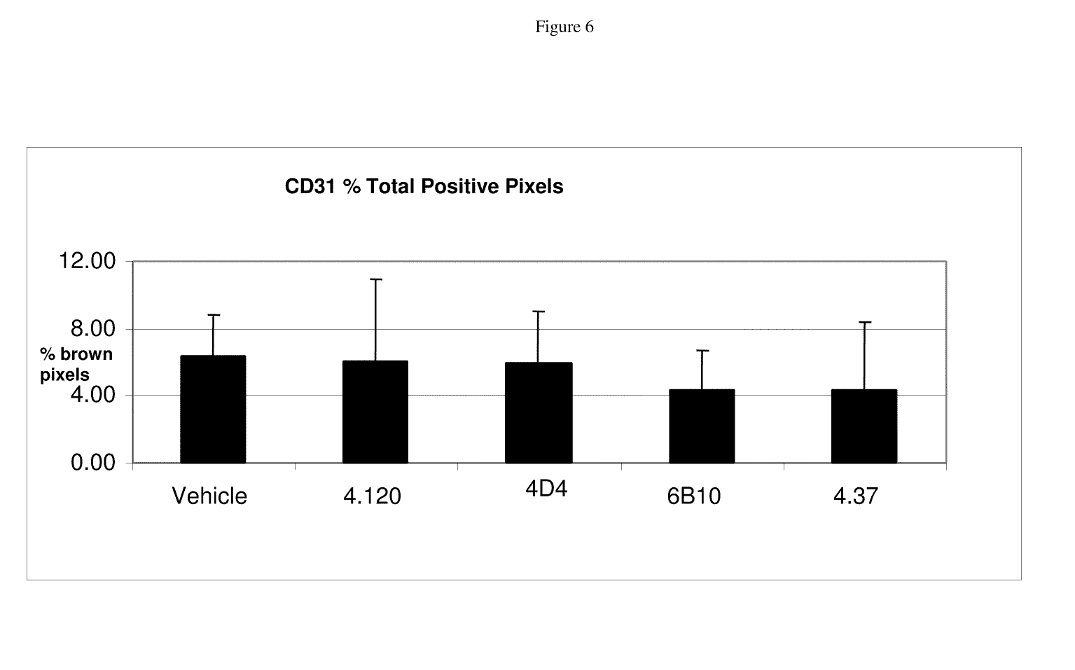

As shown in FIG. 5, results of this study demonstrate mAbs 4D4, 6B10, 4.120, and 4.37 mediated a reduction in hemoglobin content. Antibodies 6B10 and 4.37 also mediated a reduction in CD31 staining (FIG. 6), implying these antibodies exhibit anti-angiogenic activity in vivo.

### Example 10

### Structural Analysis of CD105 Antibodies

The variable heavy chains and the variable light chains of the antibodies were sequenced to determine their DNA sequences. The complete sequence information for the anti-CD105 antibodies is provided in the sequence listing with nucleotide and amino acid sequences for each gamma and kappa chain combination. The variable heavy sequences were analyzed to determine the VH family, the D-region sequence and the J-region sequence. The sequences were then translated to determine the primary amino acid sequence and compared to the germline VH, D and J-region sequences to assess somatic hypermutations.

Table 2 is a table comparing the antibody heavy chain regions to their cognate germline heavy chain region and the antibody kappa light chain regions to their cognate germ line light chain region. It should also be appreciated that where a particular antibody differs from its respective germline sequence at the amino acid level, the antibody sequence can be mutated back to the germline sequence. Such corrective mutations can occur at one, two, three or more positions, or a combination of any of the mutated positions, using standard molecular biological techniques. By way of non-limiting example, Table 5 shows that the heavy chain sequence of 4.37 (SEQ ID NO.: 26) differs from the corresponding germline sequence (see Table 2) through a D to an S at position 31 (mutation 1) and an F to an Y at position 102 (mutation 2). Thus, the amino acid or nucleotide sequence encoding the heavy chain of 4.37 can be modified at any or all of these sites. Tables 5-9 below illustrate the positions of such variations from the germline for 4.37, 6B10, 4.120. Each row represents a unique combination of germline and non-germline residues at the position indicated by bold type.

In another embodiment, the invention includes replacing any structural liabilities in the sequence that might affect the heterogeneity of the antibodies of the invention. Such liabilities include glycosylation sites, un-paired cysteines, surface exposed methionines, etc. To reduce the risk of such heterogeneity it is proposed that changes are made to remove one or more of such structural liabilities.

An “optimized” sequence as referred to herein is an antibody sequence as disclosed in Table 2 that has been mutated such that the non-germline sequence is mutated back at one or more residues to the germline sequence and may further be modified to remove structural liabilities from the sequence such as glycosylation sites.

In some embodiments of the invention, the targeted binding agent or antibody comprises a sequence comprising SEQ ID NO.: 26. In certain embodiments, SEQ ID NO.: 26 comprises any one of the combinations of germline and non-germline residues indicated by each row of Table 5. In some embodiments, SEQ ID NO: 26 comprises any one, any two, or all two of the germline residues as indicated in Table 5. In certain embodiments, SEQ ID NO.: 26 comprises any one of the unique combinations of germline and non-germline residues indicated by each row of Table 5. In other embodiments, the targeted binding agent or antibody is derived from a germline sequence with VH3-33, D6-13 and JH6 domains, wherein one or more residues has been mutated to yield the corresponding germline residue at that position.

TABLE 6
Exemplary Mutations of 4.37 Heavy Chain (SEQ ID NO: 26) to
Germline at the Indicated Residue Number

| 31 | 102 |
|---|---|
| D | F |
| S | F |
| D | Y |
| S | Y |

In some embodiments of the invention, the targeted binding agent or antibody comprises a sequence comprising SEQ ID NO.:28. In certain embodiments, SEQ ID NO.: 28 comprises any one of the combinations of germline and non-germline residues indicated by each row of Table 6. In some embodiments, SEQ ID NO: 28 comprises any one, any two, or all two, any three, or all three, of the germline residues as indicated in Table 6. In certain embodiments, SEQ ID NO.: 28 comprises any one of the unique combinations of germline and non-germline residues indicated by each row of Table 6. In other embodiments, the targeted binding agent or antibody is derived from a germline sequence with VK A3/A19 and JK3 domains, wherein one or more residues has been mutated to yield the corresponding germline residue at that position.

TABLE 7
Exemplary Mutations of 4.37 light Chain (SEQ ID NO: 28) to
Germline at the Indicated Residue Number

| 31 | 90 | 95 |
|---|---|---|
| Y | L | R |
| H | L | R |
| Y | V | R |
| H | V | R |
| Y | L | Q |
| H | L | Q |
| Y | V | Q |
| H | V | Q |

In some embodiments of the invention, the targeted binding agent or antibody comprises a sequence comprising SEQ ID NO.: 30. In certain embodiments, SEQ ID NO.: 30 comprises any one of the combinations of germline and non-germline residues indicated by each row of Table 7. In some embodiments, SEQ ID NO: 30 comprises any one, any two, any three, any four, any five, any six, any seven, or all seven of the germline residues as indicated in Table 7. In certain embodiments, SEQ ID NO.: 30 comprises any one of the unique combinations of germline and non-germline residues indicated by each row of Table 7. In other embodiments, the targeted binding agent or antibody is derived from a germline sequence with VH3-30*01, D3-10 and JH4 domains, wherein one or more residues has been mutated to yield the corresponding germline residue at that position.

TABLE 8
Exemplary Mutations of 6B10 Heavy Chain (SEQ ID NO: 30)
to Germline at the Indicated Residue Number

| 2 | 23 | 31 | 34 | 49 | 57 | 109 |
|---|---|---|---|---|---|---|
| E | T | N | I | T | K | Y |
| V | T | N | I | T | K | Y |
| E | A | N | I | T | K | Y |
| V | A | N | I | T | K | Y |
| E | T | S | I | T | K | Y |
| V | T | S | I | T | K | Y |
| E | A | S | I | T | K | Y |
| V | A | S | I | T | K | Y |
| E | T | N | M | T | K | Y |
| V | T | N | M | T | K | Y |
| E | A | N | M | T | K | Y |
| V | A | N | M | T | K | Y |
| E | T | S | M | T | K | Y |
| V | T | S | M | T | K | Y |
| E | A | S | M | T | K | Y |
| V | A | S | M | T | K | Y |
| E | T | N | I | A | K | Y |
| V | T | N | I | A | K | Y |
| E | A | N | I | A | K | Y |
| V | A | N | I | A | K | Y |
| E | T | S | I | A | K | Y |
| V | T | S | I | A | K | Y |
| E | A | S | I | A | K | Y |
| V | A | S | I | A | K | Y |
| E | T | N | M | A | K | Y |
| V | T | N | M | A | K | Y |
| E | A | N | M | A | K | Y |
| V | A | N | M | A | K | Y |
| E | T | S | M | A | K | Y |
| V | T | S | M | A | K | Y |
| E | A | S | M | A | K | Y |
| V | A | S | M | A | K | Y |
| E | T | N | I | T | N | Y |
| V | T | N | I | T | N | Y |
| E | A | N | I | T | N | Y |
| V | A | N | I | T | N | Y |
| E | T | S | I | T | N | Y |
| V | T | S | I | T | N | Y |
| E | A | S | I | T | N | Y |
| V | A | S | I | T | N | Y |
| E | T | N | M | T | N | Y |
| V | T | N | M | T | N | Y |
| E | A | N | M | T | N | Y |
| V | A | N | M | T | N | Y |
| E | T | S | M | T | N | Y |
| V | T | S | M | T | N | Y |
| E | A | S | M | T | N | Y |
| V | A | S | M | T | N | Y |
| E | T | N | I | A | N | Y |
| V | T | N | I | A | N | Y |
| E | A | N | I | A | N | Y |
| V | A | N | I | A | N | Y |
| E | T | S | I | A | N | Y |
| V | T | S | I | A | N | Y |
| E | A | S | I | A | N | Y |
| V | A | S | I | A | N | Y |
| E | T | N | M | A | N | Y |
| V | T | N | M | A | N | Y |
| E | A | N | M | A | N | Y |
| V | A | N | M | A | N | Y |
| E | T | S | M | A | N | Y |
| V | T | S | M | A | N | Y |
| E | A | S | M | A | N | Y |
| V | A | S | M | A | N | Y |
| E | T | N | I | T | K | H |
| V | T | N | I | T | K | H |
| E | A | N | I | T | K | H |
| V | A | N | I | T | K | H |
| E | T | S | I | T | K | H |
| V | T | S | I | T | K | H |
| E | A | S | I | T | K | H |
| V | A | S | I | T | K | H |
| E | T | N | M | T | K | H |
| V | T | N | M | T | K | H |
| E | A | N | M | T | K | H |
| V | A | N | M | T | K | H |
| E | T | S | M | T | K | H |
| V | T | S | M | T | K | H |
| E | A | S | M | T | K | H |
| V | A | S | M | T | K | H |
| E | T | N | I | A | K | H |
| V | T | N | I | A | K | H |
| E | A | N | I | A | K | H |
| V | A | N | I | A | K | H |
| E | T | S | I | A | K | H |
| V | T | S | I | A | K | H |
| E | A | S | I | A | K | H |
| V | A | S | I | A | K | H |
| E | T | N | M | A | K | H |
| V | T | N | M | A | K | H |
| E | A | N | M | A | K | H |
| V | A | N | M | A | K | H |
| E | T | S | M | A | K | H |
| V | T | S | M | A | K | H |
| E | A | S | M | A | K | H |
| V | A | S | M | A | K | H |
| E | T | N | I | T | N | H |
| V | T | N | I | T | N | H |
| E | A | N | I | T | N | H |
| V | A | N | I | T | N | H |
| E | T | S | I | T | N | H |
| V | T | S | I | T | N | H |
| E | A | S | I | T | N | H |
| V | A | S | I | T | N | H |
| E | T | N | M | T | N | H |
| V | T | N | M | T | N | H |
| E | A | N | M | T | N | H |
| V | A | N | M | T | N | H |
| E | T | S | M | T | N | H |
| V | T | S | M | T | N | H |
| E | A | S | M | T | N | H |
| V | A | S | M | T | N | H |
| E | T | N | I | A | N | H |
| V | T | N | I | A | N | H |
| E | A | N | I | A | N | H |
| V | A | N | I | A | N | H |
| E | T | S | I | A | N | H |
| V | T | S | I | A | N | H |
| E | A | S | I | A | N | H |
| V | A | S | I | A | N | H |
| E | T | N | M | A | N | H |
| V | T | N | M | A | N | H |
| E | A | N | M | A | N | H |
| V | A | N | M | A | N | H |
| E | T | S | M | A | N | H |
| V | T | S | M | A | N | H |
| E | A | S | M | A | N | H |
| V | A | S | M | A | N | H |
| E | T | N | I | T | K | Y |
| V | T | N | I | T | K | Y |
| E | A | N | I | T | K | Y |
| V | A | N | I | T | K | Y |
| E | T | S | I | T | K | Y |
| V | T | S | I | T | K | Y |
| E | A | S | I | T | K | Y |
| V | A | S | I | T | K | Y |
| E | T | N | M | T | K | Y |
| V | T | N | M | T | K | Y |
| E | A | N | M | T | K | Y |
| V | A | N | M | T | K | Y |
| E | T | S | M | T | K | Y |
| V | T | S | M | T | K | Y |
| E | A | S | M | T | K | Y |
| V | A | S | M | T | K | Y |
| E | T | N | I | A | K | Y |
| V | T | N | I | A | K | Y |
| E | A | N | I | A | K | Y |
| V | A | N | I | A | K | Y |
| E | T | S | I | A | K | Y |
| V | T | S | I | A | K | Y |
| E | A | S | I | A | K | Y |
| V | A | S | I | A | K | Y |
| E | T | N | M | A | K | Y |
| V | T | N | M | A | K | Y |
| E | A | N | M | A | K | Y |
| V | A | N | M | A | K | Y |
| E | T | S | M | A | K | Y |
| V | T | S | M | A | K | Y |
| E | A | S | M | A | K | Y |
| V | A | S | M | A | K | Y |
| E | T | N | I | T | N | Y |
| V | T | N | I | T | N | Y |
| E | A | N | I | T | N | Y |
| V | A | N | I | T | N | Y |
| E | T | S | I | T | N | Y |
| V | T | S | I | T | N | Y |
| E | A | S | I | T | N | Y |
| V | A | S | I | T | N | Y |
| E | T | N | M | T | N | Y |
| V | T | N | M | T | N | Y |
| E | A | N | M | T | N | Y |
| V | A | N | M | T | N | Y |
| E | T | S | M | T | N | Y |
| V | T | S | M | T | N | Y |
| E | A | S | M | T | N | Y |
| V | A | S | M | T | N | Y |
| E | T | N | I | A | N | Y |
| V | T | N | I | A | N | Y |
| E | A | N | I | A | N | Y |
| V | A | N | I | A | N | Y |
| E | T | S | I | A | N | Y |
| V | T | S | I | A | N | Y |
| E | A | S | I | A | N | Y |
| V | A | S | I | A | N | Y |
| E | T | N | M | A | N | Y |
| V | T | N | M | A | N | Y |
| E | A | N | M | A | N | Y |
| V | A | N | M | A | N | Y |
| E | T | S | M | A | N | Y |
| V | T | S | M | A | N | Y |
| E | A | S | M | A | N | Y |
| V | A | S | M | A | N | Y |
| E | T | N | I | T | K | H |
| V | T | N | I | T | K | H |
| E | A | N | I | T | K | H |
| V | A | N | I | T | K | H |
| E | T | S | I | T | K | H |
| V | T | S | I | T | K | H |
| E | A | S | I | T | K | H |
| V | A | S | I | T | K | H |
| E | T | N | M | T | K | H |
| V | T | N | M | T | K | H |
| E | A | N | M | T | K | H |
| V | A | N | M | T | K | H |
| E | T | S | M | T | K | H |
| V | T | S | M | T | K | H |
| E | A | S | M | T | K | H |
| V | A | S | M | T | K | H |
| E | T | N | I | A | K | H |
| V | T | N | I | A | K | H |
| E | A | N | I | A | K | H |
| V | A | N | I | A | K | H |
| E | T | S | I | A | K | H |
| V | T | S | I | A | K | H |
| E | A | S | I | A | K | H |
| V | A | S | I | A | K | H |
| E | T | N | M | A | K | H |
| V | T | N | M | A | K | H |
| E | A | N | M | A | K | H |
| V | A | N | M | A | K | H |
| E | T | S | M | A | K | H |
| V | T | S | M | A | K | H |
| E | A | S | M | A | K | H |
| V | A | S | M | A | K | H |
| E | T | N | I | T | N | H |
| V | T | N | I | T | N | H |
| E | A | N | I | T | N | H |
| V | A | N | I | T | N | H |
| E | T | S | I | T | N | H |
| V | T | S | I | T | N | H |
| E | A | S | I | T | N | H |
| V | A | S | I | T | N | H |
| E | T | N | M | T | N | H |
| V | T | N | M | T | N | H |
| E | A | N | M | T | N | H |
| V | A | N | M | T | N | H |
| E | T | S | M | T | N | H |
| V | T | S | M | T | N | H |
| E | A | S | M | T | N | H |
| V | A | S | M | T | N | H |
| E | T | N | I | A | N | H |
| V | T | N | I | A | N | H |
| E | A | N | I | A | N | H |
| V | A | N | I | A | N | H |
| E | T | S | I | A | N | H |
| V | T | S | I | A | N | H |
| E | A | S | I | A | N | H |
| V | A | S | I | A | N | H |
| E | T | N | M | A | N | H |
| V | T | N | M | A | N | H |
| E | A | N | M | A | N | H |
| V | A | N | M | A | N | H |
| E | T | S | M | A | N | H |
| V | T | S | M | A | N | H |
| E | A | S | M | A | N | H |
| V | A | S | M | A | N | H |
| E | T | N | I | T | K | Y |
| V | T | N | I | T | K | Y |
| E | A | N | I | T | K | Y |
| V | A | N | I | T | K | Y |
| E | T | S | I | T | K | Y |
| V | T | S | I | T | K | Y |
| E | A | S | I | T | K | Y |
| V | A | S | I | T | K | Y |
| E | T | N | M | T | K | Y |
| V | T | N | M | T | K | Y |
| E | A | N | M | T | K | Y |
| V | A | N | M | T | K | Y |
| E | T | S | M | T | K | Y |
| V | T | S | M | T | K | Y |
| E | A | S | M | T | K | Y |
| V | A | S | M | T | K | Y |
| E | T | N | I | A | K | Y |
| V | T | N | I | A | K | Y |
| E | A | N | I | A | K | Y |
| V | A | N | I | A | K | Y |
| E | T | S | I | A | K | Y |
| V | T | S | I | A | K | Y |
| E | A | S | I | A | K | Y |
| V | A | S | I | A | K | Y |
| E | T | N | M | A | K | Y |
| V | T | N | M | A | K | Y |
| E | A | N | M | A | K | Y |
| V | A | N | M | A | K | Y |
| E | T | S | M | A | K | Y |
| V | T | S | M | A | K | Y |
| E | A | S | M | A | K | Y |
| V | A | S | M | A | K | Y |
| E | T | N | I | T | N | Y |
| V | T | N | I | T | N | Y |
| E | A | N | I | T | N | Y |
| V | A | N | I | T | N | Y |
| E | T | S | I | T | N | Y |
| V | T | S | I | T | N | Y |
| E | A | S | I | T | N | Y |
| V | A | S | I | T | N | Y |
| E | T | N | M | T | N | Y |
| V | T | N | M | T | N | Y |
| E | A | N | M | T | N | Y |
| V | A | N | M | T | N | Y |
| E | T | S | M | T | N | Y |
| V | T | S | M | T | N | Y |
| E | A | S | M | T | N | Y |
| V | A | S | M | T | N | Y |
| E | T | N | I | A | N | Y |
| V | T | N | I | A | N | Y |
| E | A | N | I | A | N | Y |
| V | A | N | I | A | N | Y |
| E | T | S | I | A | N | Y |
| V | T | S | I | A | N | Y |
| E | A | S | I | A | N | Y |
| V | A | S | I | A | N | Y |
| E | T | N | M | A | N | Y |
| V | T | N | M | A | N | Y |
| E | A | N | M | A | N | Y |
| V | A | N | M | A | N | Y |
| E | T | S | M | A | N | Y |
| V | T | S | M | A | N | Y |
| E | A | S | M | A | N | Y |
| V | A | S | M | A | N | Y |
| E | T | N | I | T | K | H |
| V | T | N | I | T | K | H |
| E | A | N | I | T | K | H |
| V | A | N | I | T | K | H |
| E | T | S | I | T | K | H |
| V | T | S | I | T | K | H |
| E | A | S | I | T | K | H |
| V | A | S | I | T | K | H |
| E | T | N | M | T | K | H |
| V | T | N | M | T | K | H |
| E | A | N | M | T | K | H |
| V | A | N | M | T | K | H |
| E | T | S | M | T | K | H |
| V | T | S | M | T | K | H |
| E | A | S | M | T | K | H |
| V | A | S | M | T | K | H |
| E | T | N | I | A | K | H |
| V | T | N | I | A | K | H |
| E | A | N | I | A | K | H |
| V | A | N | I | A | K | H |
| E | T | S | I | A | K | H |
| V | T | S | I | A | K | H |
| E | A | S | I | A | K | H |
| V | A | S | I | A | K | H |
| E | T | N | M | A | K | H |
| V | T | N | M | A | K | H |
| E | A | N | M | A | K | H |
| V | A | N | M | A | K | H |
| E | T | S | M | A | K | H |
| V | T | S | M | A | K | H |
| E | A | S | M | A | K | H |
| V | A | S | M | A | K | H |
| E | T | N | I | T | N | H |
| V | T | N | I | T | N | H |
| E | A | N | I | T | N | H |
| V | A | N | I | T | N | H |
| E | T | S | I | T | N | H |
| V | T | S | I | T | N | H |
| E | A | S | I | T | N | H |
| V | A | S | I | T | N | H |
| E | T | N | M | T | N | H |
| V | T | N | M | T | N | H |
| E | A | N | M | T | N | H |
| V | A | N | M | T | N | H |
| E | T | S | M | T | N | H |
| V | T | S | M | T | N | H |
| E | A | S | M | T | N | H |
| V | A | S | M | T | N | H |
| E | T | N | I | A | N | H |
| V | T | N | I | A | N | H |
| E | A | N | I | A | N | H |
| V | A | N | I | A | N | H |
| E | T | S | I | A | N | H |
| V | T | S | I | A | N | H |
| E | A | S | I | A | N | H |
| V | A | S | I | A | N | H |
| E | T | N | M | A | N | H |
| V | T | N | M | A | N | H |
| E | A | N | M | A | N | H |
| V | A | N | M | A | N | H |
| E | T | S | M | A | N | H |
| V | T | S | M | A | N | H |
| E | A | S | M | A | N | H |
| V | A | S | M | A | N | H |

In some embodiments of the invention, the targeted binding agent or antibody comprises a sequence comprising SEQ ID NO.: 32. In certain embodiments, SEQ ID NO.: 32 comprises any one of the combinations of germline and non-germline residues indicated by each row of Table 8. In some embodiments, SEQ ID NO: 32 comprises any one, any two, any three, any four, any five, any six, any seven, any eight, any nine or all nine of the germline residues as indicated in Table 8. In certain embodiments, SEQ ID NO.: 32 comprises any one of the unique combinations of germline and non-germline residues indicated by each row of Table 8. In other embodiments, the targeted binding agent or antibody is derived from a germline sequence with Vk, Vk08/018 and JK4 domains, wherein one or more residues has been mutated to yield the corresponding germline residue at that position.

TABLE 9
Exemplary Mutations of 6B10 light Chain (SEQ ID NO: 32)
to Germline at the Indicated Residue Number

| 30 | 31 | 32 | 39 | 45 | 83 | 85 | 87 | 103 |
|---|---|---|---|---|---|---|---|---|
| Y | K | S | R | K | F | R | F | R |
| S | K | S | R | K | F | R | F | R |
| Y | N | S | R | K | F | R | F | R |
| S | N | S | R | K | F | R | F | R |
| Y | K | Y | R | K | F | R | F | R |
| S | K | Y | R | K | F | R | F | R |
| Y | N | Y | R | K | F | R | F | R |
| S | N | Y | R | K | F | R | F | R |
| Y | K | S | K | K | F | R | F | R |
| S | K | S | K | K | F | R | F | R |
| Y | N | S | K | K | F | R | F | R |
| S | N | S | K | K | F | R | F | R |
| Y | K | Y | K | K | F | R | F | R |
| S | K | Y | K | K | F | R | F | R |
| Y | N | Y | K | K | F | R | F | R |
| S | N | Y | K | K | F | R | F | R |
| Y | K | S | R | N | F | R | F | R |
| S | K | S | R | N | F | R | F | R |
| Y | N | S | R | N | F | R | F | R |
| S | N | S | R | N | F | R | F | R |
| Y | K | Y | R | N | F | R | F | R |
| S | K | Y | R | N | F | R | F | R |
| Y | N | Y | R | N | F | R | F | R |
| S | N | Y | R | N | F | R | F | R |
| Y | K | S | K | N | F | R | F | R |
| S | K | S | K | N | F | R | F | R |
| Y | N | S | K | N | F | R | F | R |
| S | N | S | K | N | F | R | F | R |
| Y | K | Y | K | N | F | R | F | R |
| S | K | Y | K | N | F | R | F | R |
| Y | N | Y | K | N | F | R | F | R |
| S | N | Y | K | N | F | R | F | R |
| Y | K | S | R | K | I | R | F | R |
| S | K | S | R | K | I | R | F | R |
| Y | N | S | R | K | I | R | F | R |
| S | N | S | R | K | I | R | F | R |
| Y | K | Y | R | K | I | R | F | R |
| S | K | Y | R | K | I | R | F | R |
| Y | N | Y | R | K | I | R | F | R |
| S | N | Y | R | K | I | R | F | R |
| Y | K | S | K | K | I | R | F | R |
| S | K | S | K | K | I | R | F | R |
| Y | N | S | K | K | I | R | F | R |
| S | N | S | K | K | I | R | F | R |
| Y | K | Y | K | K | I | R | F | R |
| S | K | Y | K | K | I | R | F | R |
| Y | N | Y | K | K | I | R | F | R |
| S | N | Y | K | K | I | R | F | R |
| Y | K | S | R | N | I | R | F | R |
| S | K | S | R | N | I | R | F | R |
| Y | N | S | R | N | I | R | F | R |
| S | N | S | R | N | I | R | F | R |
| Y | K | Y | R | N | I | R | F | R |
| S | K | Y | R | N | I | R | F | R |
| Y | N | Y | R | N | I | R | F | R |
| S | N | Y | R | N | I | R | F | R |
| Y | K | S | K | N | I | R | F | R |
| S | K | S | K | N | I | R | F | R |
| Y | N | S | K | N | I | R | F | R |
| S | N | S | K | N | I | R | F | R |
| Y | K | Y | K | N | I | R | F | R |
| S | K | Y | K | N | I | R | F | R |
| Y | N | Y | K | N | I | R | F | R |
| S | N | Y | K | N | I | R | F | R |
| Y | K | S | R | K | F | T | F | R |
| S | K | S | R | K | F | T | F | R |
| Y | N | S | R | K | F | T | F | R |
| S | N | S | R | K | F | T | F | R |
| Y | K | Y | R | K | F | T | F | R |
| S | K | Y | R | K | F | T | F | R |
| Y | N | Y | R | K | F | T | F | R |
| S | N | Y | R | K | F | T | F | R |
| Y | K | S | K | K | F | T | F | R |
| S | K | S | K | K | F | T | F | R |
| Y | N | S | K | K | F | T | F | R |
| S | N | S | K | K | F | T | F | R |
| Y | K | Y | K | K | F | T | F | R |
| S | K | Y | K | K | F | T | F | R |
| Y | N | Y | K | K | F | T | F | R |
| S | N | Y | K | K | F | T | F | R |
| Y | K | S | R | N | F | T | F | R |
| S | K | S | R | N | F | T | F | R |
| Y | N | S | R | N | F | T | F | R |
| S | N | S | R | N | F | T | F | R |
| Y | K | Y | R | N | F | T | F | R |
| S | K | Y | R | N | F | T | F | R |
| Y | N | Y | R | N | F | T | F | R |
| S | N | Y | R | N | F | T | F | R |
| Y | K | S | K | N | F | T | F | R |
| S | K | S | K | N | F | T | F | R |
| Y | N | S | K | N | F | T | F | R |
| S | N | S | K | N | F | T | F | R |
| Y | K | Y | K | N | F | T | F | R |
| S | K | Y | K | N | F | T | F | R |
| Y | N | Y | K | N | F | T | F | R |
| S | N | Y | K | N | F | T | F | R |
| Y | K | S | R | K | I | T | F | R |
| S | K | S | R | K | I | T | F | R |
| Y | N | S | R | K | I | T | F | R |
| S | N | S | R | K | I | T | F | R |
| Y | K | Y | R | K | I | T | F | R |
| S | K | Y | R | K | I | T | F | R |
| Y | N | Y | R | K | I | T | F | R |
| S | N | Y | R | K | I | T | F | R |
| Y | K | S | K | K | I | T | F | R |
| S | K | S | K | K | I | T | F | R |
| Y | N | S | K | K | I | T | F | R |
| S | N | S | K | K | I | T | F | R |
| Y | K | Y | K | K | I | T | F | R |
| S | K | Y | K | K | I | T | F | R |
| Y | N | Y | K | K | I | T | F | R |
| S | N | Y | K | K | I | T | F | R |
| Y | K | S | R | N | I | T | F | R |
| S | K | S | R | N | I | T | F | R |
| Y | N | S | R | N | I | T | F | R |
| S | N | S | R | N | I | T | F | R |
| Y | K | Y | R | N | I | T | F | R |
| S | K | Y | R | N | I | T | F | R |
| Y | N | Y | R | N | I | T | F | R |
| S | N | Y | R | N | I | T | F | R |
| Y | K | S | K | N | I | T | F | R |
| S | K | S | K | N | I | T | F | R |
| Y | N | S | K | N | I | T | F | R |
| S | N | S | K | N | I | T | F | R |
| Y | K | Y | K | N | I | T | F | R |
| S | K | Y | K | N | I | T | F | R |
| Y | N | Y | K | N | I | T | F | R |
| S | N | Y | K | N | I | T | F | R |
| Y | K | S | R | K | F | R | Y | R |
| S | K | S | R | K | F | R | Y | R |
| Y | N | S | R | K | F | R | Y | R |
| S | N | S | R | K | F | R | Y | R |
| Y | K | Y | R | K | F | R | Y | R |
| S | K | Y | R | K | F | R | Y | R |
| Y | N | Y | R | K | F | R | Y | R |
| S | N | Y | R | K | F | R | Y | R |
| Y | K | S | K | K | F | R | Y | R |
| S | K | S | K | K | F | R | Y | R |
| Y | N | S | K | K | F | R | Y | R |
| S | N | S | K | K | F | R | Y | R |
| Y | K | Y | K | K | F | R | Y | R |
| S | K | Y | K | K | F | R | Y | R |
| Y | N | Y | K | K | F | R | Y | R |
| S | N | Y | K | K | F | R | Y | R |
| Y | K | S | R | N | F | R | Y | R |
| S | K | S | R | N | F | R | Y | R |
| Y | N | S | R | N | F | R | Y | R |
| S | N | S | R | N | F | R | Y | R |
| Y | K | Y | R | N | F | R | Y | R |
| S | K | Y | R | N | F | R | Y | R |
| Y | N | Y | R | N | F | R | Y | R |
| S | N | Y | R | N | F | R | Y | R |
| Y | K | S | K | N | F | R | Y | R |
| S | K | S | K | N | F | R | Y | R |
| Y | N | S | K | N | F | R | Y | R |
| S | N | S | K | N | F | R | Y | R |
| Y | K | Y | K | N | F | R | Y | R |
| S | K | Y | K | N | F | R | Y | R |
| Y | N | Y | K | N | F | R | Y | R |
| S | N | Y | K | N | F | R | Y | R |
| Y | K | S | R | K | I | R | Y | R |
| S | K | S | R | K | I | R | Y | R |
| Y | N | S | R | K | I | R | Y | R |
| S | N | S | R | K | I | R | Y | R |
| Y | K | Y | R | K | I | R | Y | R |
| S | K | Y | R | K | I | R | Y | R |
| Y | N | Y | R | K | I | R | Y | R |
| S | N | Y | R | K | I | R | Y | R |
| Y | K | S | K | K | I | R | Y | R |
| S | K | S | K | K | I | R | Y | R |
| Y | N | S | K | K | I | R | Y | R |
| S | N | S | K | K | I | R | Y | R |
| Y | K | Y | K | K | I | R | Y | R |
| S | K | Y | K | K | I | R | Y | R |
| Y | N | Y | K | K | I | R | Y | R |
| S | N | Y | K | K | I | R | Y | R |
| Y | K | S | R | N | I | R | Y | R |
| S | K | S | R | N | I | R | Y | R |
| Y | N | S | R | N | I | R | Y | R |
| S | N | S | R | N | I | R | Y | R |
| Y | K | Y | R | N | I | R | Y | R |
| S | K | Y | R | N | I | R | Y | R |
| Y | N | Y | R | N | I | R | Y | R |
| S | N | Y | R | N | I | R | Y | R |
| Y | K | S | K | N | I | R | Y | R |
| S | K | S | K | N | I | R | Y | R |
| Y | N | S | K | N | I | R | Y | R |
| S | N | S | K | N | I | R | Y | R |
| Y | K | Y | K | N | I | R | Y | R |
| S | K | Y | K | N | I | R | Y | R |
| Y | N | Y | K | N | I | R | Y | R |
| S | N | Y | K | N | I | R | Y | R |
| Y | K | S | R | K | F | T | Y | R |
| S | K | S | R | K | F | T | Y | R |
| Y | N | S | R | K | F | T | Y | R |
| S | N | S | R | K | F | T | Y | R |
| Y | K | Y | R | K | F | T | Y | R |
| S | K | Y | R | K | F | T | Y | R |
| Y | N | Y | R | K | F | T | Y | R |
| S | N | Y | R | K | F | T | Y | R |
| Y | K | S | K | K | F | T | Y | R |
| S | K | S | K | K | F | T | Y | R |
| Y | N | S | K | K | F | T | Y | R |
| S | N | S | K | K | F | T | Y | R |
| Y | K | Y | K | K | F | T | Y | R |
| S | K | Y | K | K | F | T | Y | R |
| Y | N | Y | K | K | F | T | Y | R |
| S | N | Y | K | K | F | T | Y | R |
| Y | K | S | R | N | F | T | Y | R |
| S | K | S | R | N | F | T | Y | R |
| Y | N | S | R | N | F | T | Y | R |
| S | N | S | R | N | F | T | Y | R |
| Y | K | Y | R | N | F | T | Y | R |
| S | K | Y | R | N | F | T | Y | R |
| Y | N | Y | R | N | F | T | Y | R |
| S | N | Y | R | N | F | T | Y | R |
| Y | K | S | K | N | F | T | Y | R |
| S | K | S | K | N | F | T | Y | R |
| Y | N | S | K | N | F | T | Y | R |
| S | N | S | K | N | F | T | Y | R |
| Y | K | Y | K | N | F | T | Y | R |
| S | K | Y | K | N | F | T | Y | R |
| Y | N | Y | K | N | F | T | Y | R |
| S | N | Y | K | N | F | T | Y | R |
| Y | K | S | R | K | I | T | Y | R |
| S | K | S | R | K | I | T | Y | R |
| Y | N | S | R | K | I | T | Y | R |
| S | N | S | R | K | I | T | Y | R |
| Y | K | Y | R | K | I | T | Y | R |
| S | K | Y | R | K | I | T | Y | R |
| Y | N | Y | R | K | I | T | Y | R |
| S | N | Y | R | K | I | T | Y | R |
| Y | K | S | K | K | I | T | Y | R |
| S | K | S | K | K | I | T | Y | R |
| Y | N | S | K | K | I | T | Y | R |
| S | N | S | K | K | I | T | Y | R |
| Y | K | Y | K | K | I | T | Y | R |
| S | K | Y | K | K | I | T | Y | R |
| Y | N | Y | K | K | I | T | Y | R |
| S | N | Y | K | K | I | T | Y | R |
| Y | K | S | R | N | I | T | Y | R |
| S | K | S | R | N | I | T | Y | R |
| Y | N | S | R | N | I | T | Y | R |
| S | N | S | R | N | I | T | Y | R |
| Y | K | Y | R | N | I | T | Y | R |
| S | K | Y | R | N | I | T | Y | R |
| Y | N | Y | R | N | I | T | Y | R |
| S | N | Y | R | N | I | T | Y | R |
| Y | K | S | K | N | I | T | Y | R |
| S | K | S | K | N | I | T | Y | R |
| Y | N | S | K | N | I | T | Y | R |
| S | N | S | K | N | I | T | Y | R |
| Y | K | Y | K | N | I | T | Y | R |
| S | K | Y | K | N | I | T | Y | R |
| Y | N | Y | K | N | I | T | Y | R |
| S | N | Y | K | N | I | T | Y | R |
| Y | K | S | R | K | F | R | F | K |
| S | K | S | R | K | F | R | F | K |
| Y | N | S | R | K | F | R | F | K |
| S | N | S | R | K | F | R | F | K |
| Y | K | Y | R | K | F | R | F | K |
| S | K | Y | R | K | F | R | F | K |
| Y | N | Y | R | K | F | R | F | K |
| S | N | Y | R | K | F | R | F | K |
| Y | K | S | K | K | F | R | F | K |
| S | K | S | K | K | F | R | F | K |
| Y | N | S | K | K | F | R | F | K |
| S | N | S | K | K | F | R | F | K |
| Y | K | Y | K | K | F | R | F | K |
| S | K | Y | K | K | F | R | F | K |
| Y | N | Y | K | K | F | R | F | K |
| S | N | Y | K | K | F | R | F | K |
| Y | K | S | R | N | F | R | F | K |
| S | K | S | R | N | F | R | F | K |
| Y | N | S | R | N | F | R | F | K |
| S | N | S | R | N | F | R | F | K |
| Y | K | Y | R | N | F | R | F | K |
| S | K | Y | R | N | F | R | F | K |
| Y | N | Y | R | N | F | R | F | K |
| S | N | Y | R | N | F | R | F | K |
| Y | K | S | K | N | F | R | F | K |
| S | K | S | K | N | F | R | F | K |
| Y | N | S | K | N | F | R | F | K |
| S | N | S | K | N | F | R | F | K |
| Y | K | Y | K | N | F | R | F | K |
| S | K | Y | K | N | F | R | F | K |
| Y | N | Y | K | N | F | R | F | K |
| S | N | Y | K | N | F | R | F | K |
| Y | K | S | R | K | I | R | F | K |
| S | K | S | R | K | I | R | F | K |
| Y | N | S | R | K | I | R | F | K |
| S | N | S | R | K | I | R | F | K |
| Y | K | Y | R | K | I | R | F | K |
| S | K | Y | R | K | I | R | F | K |
| Y | N | Y | R | K | I | R | F | K |
| S | N | Y | R | K | I | R | F | K |
| Y | K | S | K | K | I | R | F | K |
| S | K | S | K | K | I | R | F | K |
| Y | N | S | K | K | I | R | F | K |
| S | N | S | K | K | I | R | F | K |
| Y | K | Y | K | K | I | R | F | K |
| S | K | Y | K | K | I | R | F | K |
| Y | N | Y | K | K | I | R | F | K |
| S | N | Y | K | K | I | R | F | K |
| Y | K | S | R | N | I | R | F | K |
| S | K | S | R | N | I | R | F | K |
| Y | N | S | R | N | I | R | F | K |
| S | N | S | R | N | I | R | F | K |
| Y | K | Y | R | N | I | R | F | K |
| S | K | Y | R | N | I | R | F | K |
| Y | N | Y | R | N | I | R | F | K |
| S | N | Y | R | N | I | R | F | K |
| Y | K | S | K | N | I | R | F | K |
| S | K | S | K | N | I | R | F | K |
| Y | N | S | K | N | I | R | F | K |
| S | N | S | K | N | I | R | F | K |
| Y | K | Y | K | N | I | R | F | K |
| S | K | Y | K | N | I | R | F | K |
| Y | N | Y | K | N | I | R | F | K |
| S | N | Y | K | N | I | R | F | K |
| Y | K | S | R | K | F | T | F | K |
| S | K | S | R | K | F | T | F | K |
| Y | N | S | R | K | F | T | F | K |
| S | N | S | R | K | F | T | F | K |
| Y | K | Y | R | K | F | T | F | K |
| S | K | Y | R | K | F | T | F | K |
| Y | N | Y | R | K | F | T | F | K |
| S | N | Y | R | K | F | T | F | K |
| Y | K | S | K | K | F | T | F | K |
| S | K | S | K | K | F | T | F | K |
| Y | N | S | K | K | F | T | F | K |
| S | N | S | K | K | F | T | F | K |
| Y | K | Y | K | K | F | T | F | K |
| S | K | Y | K | K | F | T | F | K |
| Y | N | Y | K | K | F | T | F | K |
| S | N | Y | K | K | F | T | F | K |
| Y | K | S | R | N | F | T | F | K |
| S | K | S | R | N | F | T | F | K |
| Y | N | S | R | N | F | T | F | K |
| S | N | S | R | N | F | T | F | K |
| Y | K | Y | R | N | F | T | F | K |
| S | K | Y | R | N | F | T | F | K |
| Y | N | Y | R | N | F | T | F | K |
| S | N | Y | R | N | F | T | F | K |
| Y | K | S | K | N | F | T | F | K |
| S | K | S | K | N | F | T | F | K |
| Y | N | S | K | N | F | T | F | K |
| S | N | S | K | N | F | T | F | K |
| Y | K | Y | K | N | F | T | F | K |
| S | K | Y | K | N | F | T | F | K |
| Y | N | Y | K | N | F | T | F | K |
| S | N | Y | K | N | F | T | F | K |
| Y | K | S | R | K | I | T | F | K |
| S | K | S | R | K | I | T | F | K |
| Y | N | S | R | K | I | T | F | K |
| S | N | S | R | K | I | T | F | K |
| Y | K | Y | R | K | I | T | F | K |
| S | K | Y | R | K | I | T | F | K |
| Y | N | Y | R | K | I | T | F | K |
| S | N | Y | R | K | I | T | F | K |
| Y | K | S | K | K | I | T | F | K |
| S | K | S | K | K | I | T | F | K |
| Y | N | S | K | K | I | T | F | K |
| S | N | S | K | K | I | T | F | K |
| Y | K | Y | K | K | I | T | F | K |
| S | K | Y | K | K | I | T | F | K |
| Y | N | Y | K | K | I | T | F | K |
| S | N | Y | K | K | I | T | F | K |
| Y | K | S | R | N | I | T | F | K |
| S | K | S | R | N | I | T | F | K |
| Y | N | S | R | N | I | T | F | K |
| S | N | S | R | N | I | T | F | K |
| Y | K | Y | R | N | I | T | F | K |
| S | K | Y | R | N | I | T | F | K |
| Y | N | Y | R | N | I | T | F | K |
| S | N | Y | R | N | I | T | F | K |
| Y | K | S | K | N | I | T | F | K |
| S | K | S | K | N | I | T | F | K |
| Y | N | S | K | N | I | T | F | K |
| S | N | S | K | N | I | T | F | K |
| Y | K | Y | K | N | I | T | F | K |
| S | K | Y | K | N | I | T | F | K |
| Y | N | Y | K | N | I | T | F | K |
| S | N | Y | K | N | I | T | F | K |
| Y | K | S | R | K | F | R | Y | K |
| S | K | S | R | K | F | R | Y | K |
| Y | N | S | R | K | F | R | Y | K |
| S | N | S | R | K | F | R | Y | K |
| Y | K | Y | R | K | F | R | Y | K |
| S | K | Y | R | K | F | R | Y | K |
| Y | N | Y | R | K | F | R | Y | K |
| S | N | Y | R | K | F | R | Y | K |
| Y | K | S | K | K | F | R | Y | K |
| S | K | S | K | K | F | R | Y | K |
| Y | N | S | K | K | F | R | Y | K |
| S | N | S | K | K | F | R | Y | K |
| Y | K | Y | K | K | F | R | Y | K |
| S | K | Y | K | K | F | R | Y | K |
| Y | N | Y | K | K | F | R | Y | K |
| S | N | Y | K | K | F | R | Y | K |
| Y | K | S | R | N | F | R | Y | K |
| S | K | S | R | N | F | R | Y | K |
| Y | N | S | R | N | F | R | Y | K |
| S | N | S | R | N | F | R | Y | K |
| Y | K | Y | R | N | F | R | Y | K |
| S | K | Y | R | N | F | R | Y | K |
| Y | N | Y | R | N | F | R | Y | K |
| S | N | Y | R | N | F | R | Y | K |
| Y | K | S | K | N | F | R | Y | K |
| S | K | S | K | N | F | R | Y | K |
| Y | N | S | K | N | F | R | Y | K |
| S | N | S | K | N | F | R | Y | K |
| Y | K | Y | K | N | F | R | Y | K |
| S | K | Y | K | N | F | R | Y | K |
| Y | N | Y | K | N | F | R | Y | K |
| S | N | Y | K | N | F | R | Y | K |
| Y | K | S | R | K | I | R | Y | K |
| S | K | S | R | K | I | R | Y | K |
| Y | N | S | R | K | I | R | Y | K |
| S | N | S | R | K | I | R | Y | K |
| Y | K | Y | R | K | I | R | Y | K |
| S | K | Y | R | K | I | R | Y | K |
| Y | N | Y | R | K | I | R | Y | K |
| S | N | Y | R | K | I | R | Y | K |
| Y | K | S | K | K | I | R | Y | K |
| S | K | S | K | K | I | R | Y | K |
| Y | N | S | K | K | I | R | Y | K |
| S | N | S | K | K | I | R | Y | K |
| Y | K | Y | K | K | I | R | Y | K |
| S | K | Y | K | K | I | R | Y | K |
| Y | N | Y | K | K | I | R | Y | K |
| S | N | Y | K | K | I | R | Y | K |
| Y | K | S | R | N | I | R | Y | K |
| S | K | S | R | N | I | R | Y | K |
| Y | N | S | R | N | I | R | Y | K |
| S | N | S | R | N | I | R | Y | K |
| Y | K | Y | R | N | I | R | Y | K |
| S | K | Y | R | N | I | R | Y | K |
| Y | N | Y | R | N | I | R | Y | K |
| S | N | Y | R | N | I | R | Y | K |
| Y | K | S | K | N | I | R | Y | K |
| S | K | S | K | N | I | R | Y | K |
| Y | N | S | K | N | I | R | Y | K |
| S | N | S | K | N | I | R | Y | K |
| Y | K | Y | K | N | I | R | Y | K |
| S | K | Y | K | N | I | R | Y | K |
| Y | N | Y | K | N | I | R | Y | K |
| S | N | Y | K | N | I | R | Y | K |
| Y | K | S | R | K | F | T | Y | K |
| S | K | S | R | K | F | T | Y | K |
| Y | N | S | R | K | F | T | Y | K |
| S | N | S | R | K | F | T | Y | K |
| Y | K | Y | R | K | F | T | Y | K |
| S | K | Y | R | K | F | T | Y | K |
| Y | N | Y | R | K | F | T | Y | K |
| S | N | Y | R | K | F | T | Y | K |
| Y | K | S | K | K | F | T | Y | K |
| S | K | S | K | K | F | T | Y | K |
| Y | N | S | K | K | F | T | Y | K |
| S | N | S | K | K | F | T | Y | K |
| Y | K | Y | K | K | F | T | Y | K |
| S | K | Y | K | K | F | T | Y | K |
| Y | N | Y | K | K | F | T | Y | K |
| S | N | Y | K | K | F | T | Y | K |
| Y | K | S | R | N | F | T | Y | K |
| S | K | S | R | N | F | T | Y | K |
| Y | N | S | R | N | F | T | Y | K |
| S | N | S | R | N | F | T | Y | K |
| Y | K | Y | R | N | F | T | Y | K |
| S | K | Y | R | N | F | T | Y | K |
| Y | N | Y | R | N | F | T | Y | K |
| S | N | Y | R | N | F | T | Y | K |
| Y | K | S | K | N | F | T | Y | K |
| S | K | S | K | N | F | T | Y | K |
| Y | N | S | K | N | F | T | Y | K |
| S | N | S | K | N | F | T | Y | K |
| Y | K | Y | K | N | F | T | Y | K |
| S | K | Y | K | N | F | T | Y | K |
| Y | N | Y | K | N | F | T | Y | K |
| S | N | Y | K | N | F | T | Y | K |
| Y | K | S | R | K | I | T | Y | K |
| S | K | S | R | K | I | T | Y | K |
| Y | N | S | R | K | I | T | Y | K |
| S | N | S | R | K | I | T | Y | K |
| Y | K | Y | R | K | I | T | Y | K |
| S | K | Y | R | K | I | T | Y | K |
| Y | N | Y | R | K | I | T | Y | K |
| S | N | Y | R | K | I | T | Y | K |
| Y | K | S | K | K | I | T | Y | K |
| S | K | S | K | K | I | T | Y | K |
| Y | N | S | K | K | I | T | Y | K |
| S | N | S | K | K | I | T | Y | K |
| Y | K | Y | K | K | I | T | Y | K |
| S | K | Y | K | K | I | T | Y | K |
| Y | N | Y | K | K | I | T | Y | K |
| S | N | Y | K | K | I | T | Y | K |
| Y | K | S | R | N | I | T | Y | K |
| S | K | S | R | N | I | T | Y | K |
| Y | N | S | R | N | I | T | Y | K |
| S | N | S | R | N | I | T | Y | K |
| Y | K | Y | R | N | I | T | Y | K |
| S | K | Y | R | N | I | T | Y | K |
| Y | N | Y | R | N | I | T | Y | K |
| S | N | Y | R | N | I | T | Y | K |
| Y | K | S | K | N | I | T | Y | K |
| S | K | S | K | N | I | T | Y | K |
| Y | N | S | K | N | I | T | Y | K |
| S | N | S | K | N | I | T | Y | K |
| Y | K | Y | K | N | I | T | Y | K |
| S | K | Y | K | N | I | T | Y | K |
| Y | N | Y | K | N | I | T | Y | K |
| S | N | Y | K | N | I | T | Y | K |

In some embodiments of the invention, the targeted binding agent or antibody comprises a sequence comprising SEQ ID NO.: 2. In certain embodiments, SEQ ID NO.: 2 comprises any one of the combinations of germline and non-germline residues indicated by each row of Table 9. In some embodiments, SEQ ID NO: 2 comprises any one, any two, any three, any four, any five, any six, or all six of the germline residues as indicated in Table 9. In certain embodiments, SEQ ID NO.: 2 comprises any one of the unique combinations of germline and non-germline residues indicated by each row of Table 9. In other embodiments, the targeted binding agent or antibody is derived from a germline sequence with VH4-59, D5-12, JH4 wherein one or more residues has been mutated to yield the corresponding germline residue at that position.

TABLE 10
Exemplary Mutations of 4.120 Heavy Chain (SEQ ID NO: 2)
to Germline at the Indicated Residue Number

| 41 | 50 | 54 | 69 | 101 | 106 |
|---|---|---|---|---|---|
| A | R | T | M | G | G |
| P | R | T | M | G | G |
| A | Y | T | M | G | G |
| P | Y | T | M | G | G |
| A | R | Y | M | G | G |
| P | R | Y | M | G | G |
| A | Y | Y | M | G | G |
| P | Y | Y | M | G | G |
| A | R | T | I | G | G |
| P | R | T | I | G | G |
| A | Y | T | I | G | G |
| P | Y | T | I | G | G |
| A | R | Y | I | G | G |
| P | R | Y | I | G | G |
| A | Y | Y | I | G | G |
| P | Y | Y | I | G | G |
| A | R | T | M | V | G |
| P | R | T | M | V | G |
| A | Y | T | M | V | G |
| P | Y | T | M | V | G |
| A | R | Y | M | V | G |
| P | R | Y | M | V | G |
| A | Y | Y | M | V | G |
| P | Y | Y | M | V | G |
| A | R | T | I | V | G |
| P | R | T | I | V | G |
| A | Y | T | I | V | G |
| P | Y | T | I | V | G |
| A | R | Y | I | V | G |
| P | R | Y | I | V | G |
| A | Y | Y | I | V | G |
| P | Y | Y | I | V | G |
| A | R | T | M | G | Y |
| P | R | T | M | G | Y |
| A | Y | T | M | G | Y |
| P | Y | T | M | G | Y |
| A | R | Y | M | G | Y |
| P | R | Y | M | G | Y |
| A | Y | Y | M | G | Y |
| P | Y | Y | M | G | Y |
| A | R | T | I | G | Y |
| P | R | T | I | G | Y |
| A | Y | T | I | G | Y |
| P | Y | T | I | G | Y |
| A | R | Y | I | G | Y |
| P | R | Y | I | G | Y |
| A | Y | Y | I | G | Y |
| P | Y | Y | I | G | Y |
| A | R | T | M | V | Y |
| P | R | T | M | V | Y |
| A | Y | T | M | V | Y |
| P | Y | T | M | V | Y |
| A | R | Y | M | V | Y |
| P | R | Y | M | V | Y |
| A | Y | Y | M | V | Y |
| P | Y | Y | M | V | Y |
| A | R | T | I | V | Y |
| P | R | T | I | V | Y |
| A | Y | T | I | V | Y |
| P | Y | T | I | V | Y |
| A | R | Y | I | V | Y |
| P | R | Y | I | V | Y |
| A | Y | Y | I | V | Y |
| P | Y | Y | I | V | Y |

The skilled person will be aware that there are alternative methods of defining CDR boundaries. The starting residue of VH CDR1 in the Table 2a has been defined according to the method as described in Scaviner D, Barbie V, Ruiz M, Lefranc M-P. Protein Displays of the Human Immunoglobulin Heavy, Kappa and Lambda Variable and Joining Regions. Exp Clin Immunogenet 1999, 16:234-240. The remaining CDR boundaries in Table 2 are defined according to the Kabat definition.

All CDR boundaries in Table 2 are defined according to the Kabat definition.

### Example 11

### FACS KD Determination

The affinity of the anti-CD105 antibodies was determined by FACS. Briefly, HUVEC cells expressing CD105 were resuspended in FACS buffer (2% FBS, 0.05% NaN₃) at a concentration of approximately 5 million cells/mL. Cells were kept on ice. Purified antibodies were serially diluted in filtered 1×PBS (2×) across 11 wells in 96-well plates. The twelfth well in each row contained buffer only. Cells and 1×PBS were added to each mAb well such that the final volume was 30 μL/well and each well contained approximately 100,000 cells. Plates were placed on a plate shaker for 3 hours at 4° C., then spun and washed 3 times with PBS. A fluorochrome-labeled secondary goat α-human polyclonal antibody was added to each well in a 200 μL volume. Plates were then incubated for 40 minutes at 4° C., then spun and washed 3 times with PBS.

The Geometric Mean Fluorescence (GMF) of 10,000 cells for each mAb concentration was determined using a FACSCalibur instrument. A nonlinear plot of GMF as a function of molecular mAb concentration was fit using Scientist software using the equation:

$$F = {P}^{′} \cdot \frac{( {K}_{D} + {L}_{T} + 1 ) - \sqrt{{( ( {K}_{D} + {L}_{T} + 1 ) )}^{2} - 4 ( {L}_{T} )}}{2}$$

In the above equation, F=Geometric mean fluorescence, L_{T}=total molecular mAb concentration, P′=proportionality constant that relates arbitrary fluorescence units to bound mAb, and K_{D}=equilibrium dissociation constant. For each mAb an estimate for K_{D} was obtained as P′ and K_{D} were allowed to float freely in the nonlinear analysis. The table below lists the resulting K_{D}s for each mAb.

| mAb | K_{D} (pM) |
|---|---|
| 4D4.1 | 622.2 |
| 3C1.1 | 871.5 |
| 6A6.2 | <1150 |
| 6B1.1 | 2300 |
| 6B10.1 | <149.06 |
| 9H10.2 | <583.75 |
| 10C9.2 | <748.01 |
| 11H2.1 | 337.7 |
| 4.12 | 770.1 |
| 4.37κ | <716.79 |

### Example 12

### ADCC and CDC Activity OF CD105 Antibodies

### (1) ADCC Assay

NK enrichment from PMBCs was performed using RosetteSep® Human NK cell enrichment cocktail and protocol (StemCell Technologies Inc., Vancouver, BC). Briefly, whole blood from donors was collected in heparinized or EDTA coated tubes and incubated with 2.25 ml of RosetteSep® Human NK Cell Enrichment cocktail (StemCell Technologies Inc., Vancouver, BC) for 20 minutes at room temperature per RosetteSep® protocol. Samples were then diluted with equal volume of PBS containing 2% FBS and 30 mL blood mixture was layered over 15 mL Ficoll (Amersham Biosciences, conical tubes. Tubes were centrifuged at 2150 rpm for 30 minutes at room temperature and the interface layer was transferred to clean 50 ml conical tubes. PBS containing 2% FBS was added followed by centrifugation for 10 minutes at 1200 rpm. Supernatants were discarded and pellets were resuspended in 1 ml PBS and stored on ice. Cells were counted using a hemacytometer and the concentration of NK cells per ml in solution was determined

Calcein-AM is the cell-permeable version of calcein. When Calcein-AM permeates into the cytoplasm, it is hydrolyzed by esterases in cells to calcein that is retained inside the cell. Viability assays using calcein are reliable and correlate well with the standard ⁵¹Cr-release assay. Briefly, target cells (HUVEC cells) were harvested and resuspended in media at 1×10⁶ cells/ml. Calcein-AM (Sigma C1359) was added to a final concentration of 10 μM (5 μl in 2 mL cells). Cells were incubated for 45 minutes at 37° C. Cells were then spun at 1200 RPM for 10 minutes, supernatants discarded, and pellets resuspended in fresh growth media (2×). Pellets were resuspended to 10,000 cells per 75 μl of growth media. Target cells were plated in 75 μl (10,000 cells/well) in round bottom plates. Antibodies were then added to target cells at 10 μg/ml in 50 μl/well diluted in media and allowed to incubate for 30 minutes at room temperature. Following incubation, 75 μl of effector cells were added at 100,000 cells/well and allowed to incubate for 4 hours at 37° C. Following incubation, plates were spun at 1200 RPM for 5 minutes. Supernatants (100 μL) were transferred to flat, black, clear bottom plates (Costar, cat. no. 3603) and fluorescence measured. Digitonin was used as a positive control to measure maximal calecein release.

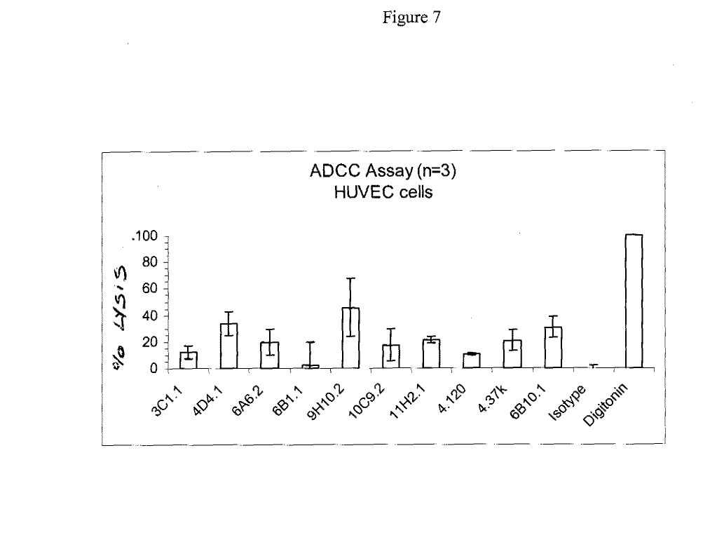

Data (shown in FIG. 7) indicate that all ten mAbs profiled exhibit ADCC activity, with mAbs 4D4, 91110, and 6B10 exhibiting the highest level of lytic activity.

### (2) CDC Assay

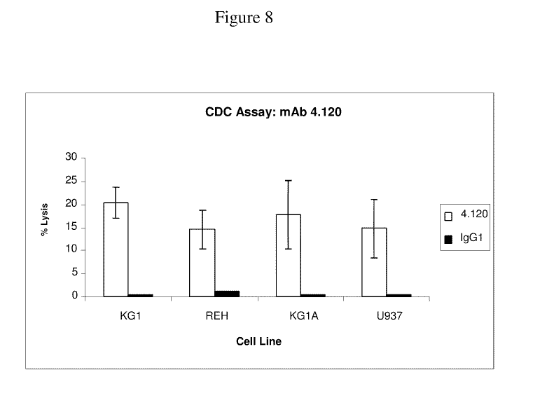

As expression of CD105 has been reported in leukemic cells (Haruta, Y. et al, 1986, PNAS, 83:7898-7902), we profiled the anti-CD105 mAbs for complement activity across several leukemia cell lines. In brief, leukemia cell lines (KG1, REH, KG1a, U937) were plated into Costar 96-well flat bottom plates (Corning Inc. Life Sciences, Lowell, Mass.) at 100,000 cells per well. The ten anti-CD105 mAbs (10 μg/ml) was added in tissue culture media and allowed to incubate at room temperature for 10 minutes. Normal human serum was added at a concentration between 10 to 50% and diluted with growth media. Serum was allowed to incubate at 37° C. for 1 hour. CellTiter Glo reagent (Promega Corp., Madison, Wis.) was added to cells and allowed to incubate for 10 minutes at room temperature in the dark and read per protocol instructions. Data (FIG. 8) indicate only mAb 4.120 elicited complement activity across all leukemic cell lines examined.

### Example 13

### Antibody Internalization of CD105 Antibodies

Antibody internalization studies were conducted to determine whether the anti-CD105 mAbs could induce internalization of CD105 in HUVEC cells. The following internalization assay was performed. HUVEC cells were aliquoted at 300,000 cells per reaction and incubated with 10 μg/ml of each anti-CD105 mAb for 1 hour at 4° C. Cells were washed twice with FACS buffer (2% FCS in PBS) and were subsequently incubated for 45 minutes at 4° C. in FACS buffer with 5 μg/ml goat Fab anti-human (Heavy+Light chain) secondary antibody conjugated to FITC. HUVEC cells were then washed once with 200 μL of FACs buffer. Two tubes of each sample were incubated for 1 hour at 4° C. and one tube at 37° C., after which cells were washed once with 200 μL FACS buffer. Then, 100 μL of 200 mM Tris (2-carboxyethyl) phosphine hydrochloride (TCEP) was added to one sample at 4° C. and one sample at 37° C. and the samples were incubated for 30 minutes at 4° C. Finally, the cells were washed once with FACS buffer and read by flow cytometry. The percent internalization was determined from the geo-means by the following equation: Percent Internalization=((37° C.+TCEP)−(4° C.+TCEP))/((4° C.−TCEP)−(4° C.+TCEP))×[100].

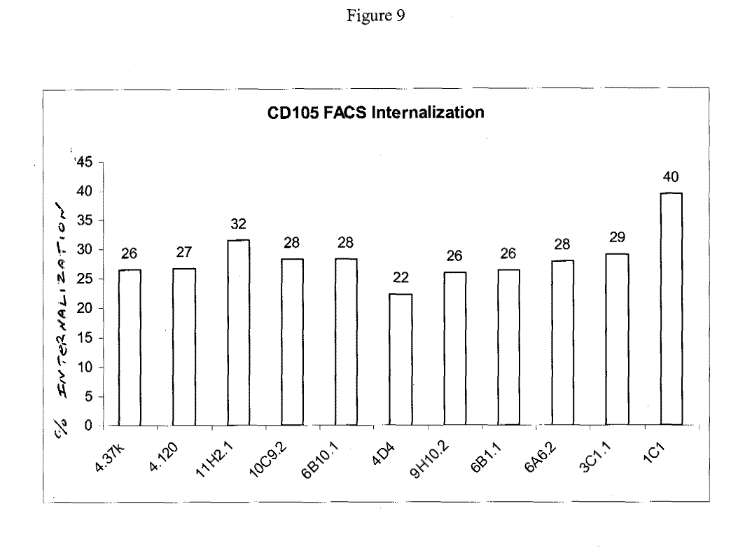

The results indicate that all the anti-CD105 mAbs mediate internalization and that approximately 25 to 30% of the cell surface antibody was internalized through its interaction with CD105 within one hour (see FIG. 9). The rapidly internalizing 1C1 antibody was used as a positive control. (Jackson, D. et al., 2008, Cancer Res., 68: 9367-74). Results indicate approximately 40% of the cell surface antibody was internalized with the 1C1 antibody.

Thus, these data suggest that the above-described anti-CD105 mAbs may be effective agents as immuno-conjugates for the delivery of toxins to cells expressing the CD105 antigen.

### INCORPORATION BY REFERENCE

All references cited herein, including patents, patent applications, papers, text books, and the like, and the references cited therein, to the extent that they are not already, are hereby incorporated herein by reference in their entirety.

### EQUIVALENTS

The foregoing written specification is considered to be sufficient to enable one skilled in the art to practice the invention. The foregoing description and Examples detail certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

What is claimed is:

1. An antibody, or binding fragment thereof, that specifically binds to CD105, wherein the antibody exhibits one or more of the following properties, comprising:
binds human CD105 with a K_{D} of less than 1 nM;
inhibits cell proliferation of HUVEC cells by greater than 5%;
increases SMAD2 phosphorylation;
exhibits anti-angiogenic activity; and
exhibits ADCC activity.

2. The antibody according to claim 1, wherein the antibody inhibits tumor growth and/or metastasis in a mammal.

3. The antibody according to claim 1, wherein the antibody binds CD105 with a Kd of less than 500 pM.

4. The antibody according to claim 1, wherein the antibody is any one of 4.120, 9H10, 10C9, 4D4, 11H2, 6B1, 4.37, 6B10, 3C1, or 6A6.

5. The antibody according to claim 4, wherein the antibody is monoclonal antibody 4.120, 4.37 or 6B10.

6. The antibody of claim 6, wherein said binding fragment is selected from the group consisting of a Fab, Fab′, F(ab′)₂, Fv and dAb fragment.

7. The antibody according to claim 1, wherein the antibody comprises:
a sequence of SEQ ID NO.: 26, wherein SEQ ID NO.:26 comprises any one of the unique combinations of germline and non-germline residues indicated by each row of Table 6;
a sequence of SEQ ID NO.: 28, and wherein SEQ ID NO.: 28 comprises any one of the unique combinations of germline and non-germline residues indicated by each row of Table 7;
a sequence of SEQ ID NO.: 30, wherein SEQ ID NO.:30 comprises any one of the unique combinations of germline and non-germline residues indicated by each row of Table 8; or
a sequence of SEQ ID NO.: 32, wherein SEQ ID NO.:32 comprises any one of the unique combinations of germline and non-germline residues indicated by each row of Table 9.

8. The antibody according to claim 1 comprising:
a CDR3 sequence as shown in Table 2;
any one of a CDR1, a CDR2 or a CDR3 sequence as shown in Table 2;
a CDR1, a CDR2 and a CDR3 sequence of a variable light chain sequence as shown in Table 2; or
a CDR1, a CDR2 and a CDR3 sequence of a variable heavy chain sequence as shown as shown in Table 2.

9. An antibody that immunospecifically binds to CD105 and comprises:
(a) a VH CDR1 of SEQ ID NO:2 having an amino acid sequence identical to or comprising 1, 2, or 3 amino acid residue substitutions relative to the VH CDR1 of SEQ ID NO:2;
(b) a VH CDR2 of SEQ ID NO:2 having an amino acid sequence identical to or comprising 1, 2, or 3 amino acid residue substitutions relative to the VH CDR2 of SEQ ID NO:2;
(c) a VH CDR3 of SEQ ID NO:2 having an amino acid sequence identical to or comprising 1, 2, or 3 amino acid residue substitutions relative to the VH CDR3 of SEQ ID NO:2;
(d) a VL CDR1 of SEQ ID NO:4 having an amino acid sequence identical to or comprising 1, 2, or 3 amino acid residue substitutions relative to VL CDR1 of SEQ ID NO:4;
(e) a VL CDR2 of SEQ ID NO:4 having an amino acid sequence identical to or comprising 1, 2, or 3 amino acid residue substitutions relative to the VL CDR2 of SEQ ID NO:4; and
(f) a VL CDR3 of SEQ ID NO:4 having an amino acid sequence identical to or comprising 1, 2, or 3 amino acid residue substitutions relative to the VL CDR3 of SEQ ID NO:4.

10. The antibody according to claim 9, wherein the antibody comprises:
(a) a VH CDR1, CDR2 and CDR3 of SEQ ID NO:2; and
(b) a VL CDR1 CDR2 and CDR3 of SEQ ID NO:4.

11. An antibody that immunospecifically binds CD105 and comprises a heavy chain variable domain having at least 90% identity to the amino acid of SEQ ID NO:2 and comprises a light chain variable domain having at least 90% identity to the amino acid sequence of SEQ ID NO:4.

12. An antibody that immunospecifically binds to CD105 and comprises:
(a) a VH CDR1 of SEQ ID NO:26 having an amino acid sequence identical to or comprising 1, 2, or 3 amino acid residue substitutions relative to the VH CDR1 of SEQ ID NO:26;
(b) a VH CDR2 of SEQ ID NO:26 having an amino acid sequence identical to or comprising 1, 2, or 3 amino acid residue substitutions relative to the VH CDR2 of SEQ ID NO:26;
(c) a VH CDR3 of SEQ ID NO:26 having an amino acid sequence identical to or comprising 1, 2, or 3 amino acid residue substitutions relative to the VH CDR3 of SEQ ID NO:26;
(d) a VL CDR1 of SEQ ID NO:28 having an amino acid sequence identical to or comprising 1, 2, or 3 amino acid residue substitutions relative to VL CDR1 of SEQ ID NO:28;
(e) a VL CDR2 of SEQ ID NO:28 having an amino acid sequence identical to or comprising 1, 2, or 3 amino acid residue substitutions relative to the VL CDR2 of SEQ ID NO:28; and
(f) a VL CDR3 of SEQ ID NO:28 having an amino acid sequence identical to or comprising 1, 2, or 3 amino acid residue substitutions relative to the VL CDR3 of SEQ ID NO:28.

13. The antibody according to claim 12, wherein the antibody comprises:
(a) a VH CDR1, CDR2 and CDR3 of SEQ ID NO:26; and
(b) a VL CDR1 CDR2 and CDR3 of SEQ ID NO:28.

14. An antibody that immunospecifically binds to CD105 and comprises:
(a) a VH CDR1 of SEQ ID NO:30 having an amino acid sequence identical to or comprising 1, 2, or 3 amino acid residue substitutions relative to the VH CDR1 of SEQ ID NO:30;
(b) a VH CDR2 of SEQ ID NO:30 having an amino acid sequence identical to or comprising 1, 2, or 3 amino acid residue substitutions relative to the VH CDR2 of SEQ ID NO:30;
(c) a VH CDR3 of SEQ ID NO:30 having an amino acid sequence identical to or comprising 1, 2, or 3 amino acid residue substitutions relative to the VH CDR3 of SEQ ID NO:30;
(d) a VL CDR1 of SEQ ID NO:32 having an amino acid sequence identical to or comprising 1, 2, or 3 amino acid residue substitutions relative to VL CDR1 of SEQ ID NO:32;
(e) a VL CDR2 of SEQ ID NO:32 having an amino acid sequence identical to or comprising 1, 2, or 3 amino acid residue substitutions relative to the VL CDR2 of SEQ ID NO:32; and
(f) a VL CDR3 of SEQ ID NO:32 having an amino acid sequence identical to or comprising 1, 2, or 3 amino acid residue substitutions relative to the VL CDR3 of SEQ ID NO:32.

15. The antibody according to claim 14, wherein the antibody comprises:
(a) a VH CDR1, CDR2 and CDR3 of SEQ ID NO:30; and
(b) a VL CDR1 CDR2 and CDR3 of SEQ ID NO:32.

16. An antibody that immunospecifically binds CD105 and comprises a heavy chain variable domain having at least 90% identity to the amino acid of SEQ ID NO:30 and comprises a light chain variable domain having at least 90% identity to the amino acid sequence of SEQ ID NO:32.

17. A fully human monoclonal antibody that competes with any one of the following is antibodies 4.120, 9H10, 10C9, 4D4, 11H2, 6B1, 4.37, 6B10, 3C1, or 6A6 for binding to CD105.

18. A fully human monoclonal antibody that binds to the same epitope on CD105 as any one of the following antibodies 4.120, 9H10, 10C9, 4D4, 11H2, 6B1, 4.37, 6B10, 3C1, or 6A6.

19. An antibody comprising an amino acid sequence comprising any one of the following:
a variable light chain amino acid sequence comprising at least one, at least two, or at least three of the light chain CDRs encoded by the polynucleotide in plasmid designated Mab4.120VH which was deposited at the American Type Culture Collection (ATCC) under number PTA-9514;
a variable heavy chain amino acid sequence comprising at least one, at least two, or at least three of the heavy chain CDRs encoded by the polynucleotide in plasmid designated Mab4.120VL which was deposited at the American Type Culture Collection (ATCC) under number PTA-9513; or
a variable heavy chain amino acid sequence comprising at least one, at least two, or at least three of the heavy chain CDRs encoded by the polynucleotide in plasmid designated Mab4.120VH which was deposited at the American Type Culture Collection (ATCC) under number PTA-9514 and a variable light chain amino acid sequence comprising at least one, at least two, or at least three of the light chain CDRs encoded by the polynucleotide in plasmid designated Mab4.120VL which was deposited at the American Type Culture Collection (ATCC) under number PTA-9513.

20. An antibody comprising an amino acid sequence comprising:
a variable heavy chain amino acid sequence comprising at least one, at least two, or at least three of the heavy chain CDRs encoded by the polynucleotide in plasmid designated Mab6B10VH which was deposited at the American Type Culture Collection (ATCC) under number PTA-9510;
a variable light chain amino acid sequence comprising at least one, at least two, or at least three of the light chain CDRs encoded by the polynucleotide in plasmid designated Mab6B10VL which was deposited at the American Type Culture Collection (ATCC) under number PTA-9503; or
a variable heavy chain amino acid sequence comprising at least one, at least two, or at least three of the heavy chain CDRs encoded by the polynucleotide in plasmid designated Mab6B10VH which was deposited at the American Type Culture Collection (ATCC) under number PTA-9510 and a variable light chain amino acid sequence comprising at least one, at least two, or at least three of the light chain CDRs of the antibody encoded by the polynucleotide in plasmid designated Mab6B10VL which was deposited at the American Type Culture Collection (ATCC) under number PTA-9503.

21. A composition comprising the antibody according to claim 1.

22. A nucleic acid molecule encoding the antibody according to claim 1.

23. A method of treating a malignant tumor in an animal, comprising: selecting an animal in need of treatment for a malignant tumor; and administering to said animal a therapeutically effective dose of the antibody of claim 1.

24. The method of claim 23, wherein said animal is human.

25. The method of claim 23, wherein the antibody is selected from the group consisting of fully human monoclonal antibodies 4B4, 2H10, 21F7, 12A1, 17F3, 9G8, 20G8, 21H3, 1E4, 3A7, 4B3, 1D4 or 21H3RK.

27. The method of claims 23-25, wherein said malignant tumor is selected from the group consisting of: melanoma, small cell lung cancer, non-small cell lung cancer, glioma, hepatocellular (liver) carcinoma, thyroid tumor, gastric (stomach) cancer, prostate cancer, breast cancer, ovarian cancer, bladder cancer, lung cancer, glioblastoma, endometrial cancer, kidney cancer, colon cancer, pancreatic cancer, esophageal carcinoma, head and neck cancers, mesothelioma, sarcomas, biliary (cholangiocarcinoma), small bowel adenocarcinoma, pediatric malignancies and epidermoid carcinoma.


SEQUENCE LISTING (60 sequences)

SEQ ID NO: 1 - DNA, 360 bp, Homo sapiens
caggtgcagctgcaggagtcgggcccaggactggtgaagccttcggagaccctgtccctcacctgcactgtctctggtggctccatcagtagttactactggagctggatccggcagcccgccgggaagggactggagtggattgggcgtatctataccagtgggagcaccaactacaacccctccctcaagagtcgagtcaccatgtcagtagacacgtccaagaaccagttctccctgaagctgagctctgtgaccgccgcggacacggccgtgtattactgtgcgagaagggatataggggctacgattaagggctttgactactggggccagggaaccctggtcaccgtctcctca

SEQ ID NO: 2 - protein, 120 aa, Homo sapiens
QVQLQESGPGLVKPSETLSLTCTVSGGSISSYYWSWIRQPAGKGLEWIGRIYTSGSTNYNPSLKSRVTMSVDTSKNQFSLKLSSVTAADTAVYYCARRDIGATIKGFDYWGQGTLVTVSS

SEQ ID NO: 3 - DNA, 318 bp, Homo sapiens
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccccaccttcggccaagggacacgactggagattaag

SEQ ID NO: 4 - protein, 106 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPTFGQGTRLEIK

SEQ ID NO: 5 - DNA, 357 bp, Homo sapiens
caggtgcaactggtggagtctgggggaggcgtggtccagcctgggaggtccctgagactctcctgtgcagcctctggattcgccttcattaactatggcatgcactgggtccgccaggctccaggcaaggggctggactgggtggcagttatatcatatgatggtagtaataaatactatacagactccgtgaagggccgattcaccatctccagagacaactccaagaacacgctgtatctgcaaatgaacagcctgagagctgaggacacggctgtgtattactgtgcgagagatctaatgggagctaccttatttgacaactggggccagggaaccctggtcaccgtctcctca

SEQ ID NO: 6 - protein, 119 aa, Homo sapiens
QVQLVESGGGVVQPGRSLRLSCAASGFAFINYGMHWVRQAPGKGLDWVAVISYDGSNKYYTDSVKGRFTISRDNSKNTLYLQMNSLRAEDTAVYYCARDLMGATLFDNWGQGTLVTVSS

SEQ ID NO: 7 - DNA, 324 bp, Homo sapiens
tcctatgtgctgactcagccaccctcggtgtcagtggccccaggtcagacggccaggatttcctgtgggggaaacaacattggaagtaaaagtgtgcactggttccagcagaagccaggccaggcccctgtgctggtcgtctatgatgatagcgaccggccctcagggatccctgagcgattctctggttccaactctgggaacacggccaccctgaccatcagcagggtcgaagccggggatgaggccgactattactgtcaggtgtgggatagtagtagtgatcatgtggtattcggcggagggaccaagctgaccgtccta

SEQ ID NO: 8 - protein, 108 aa, Homo sapiens
SYVLTQPPSVSVAPGQTARISCGGNNIGSKSVHWFQQKPGQAPVLVVYDDSDRPSGIPERFSGSNSGNTATLTISRVEAGDEADYYCQVWDSSSDHVVFGGGTKLTVL

SEQ ID NO: 9 - DNA, 369 bp, Homo sapiens
caggtgcagctggtggagtctgggggaggcgtggtccagcctgggaggtccctgagactctcctgtgcagcgtctggattcaccttcaggagctatggcatgcactgggtccgccaggctccaggcaaggggctggagtgggtggcagttatatggtatgatggaagtaataaatactatgcagactccgtgaagggccgattcaccatctccagagacaattccaagaatacgctggatctgcaaatgaacagcctgagagccgaggacacggctgtgtattactgtgcgagagatcggatagcagcagcccgctactacaacggtatggacgtctggggccaagggaccacggtcaccgtctcctca

SEQ ID NO: 10 - protein, 123 aa, Homo sapiens
QVQLVESGGGVVQPGRSLRLSCAASGFTFRSYGMHWVRQAPGKGLEWVAVIWYDGSNKYYADSVKGRFTISRDNSKNTLDLQMNSLRAEDTAVYYCARDRIAAARYYNGMDVWGQGTTVTVSS

SEQ ID NO: 11 - DNA, 339 bp, Homo sapiens
gccatcgtgatgacccagtctccagactccctggctgtgtctctgggcgagagggccaccatcaactgcaagtccagccagagtgttttatacagctccaataataagaactacttagcttggtaccagcagaaaccaggacaacctcctaatctactcttttactgggcatctacccgggaatccggggtccctgatcgcttcagtgtcagcgggtctgggacagatttcactctcaccatcagtagcctgcaggctgaagatgtggcagtttattactgtcagcaatattatgattctccgctcactttcggcggagggaccaaggtggagatcaaa

SEQ ID NO: 12 - protein, 113 aa, Homo sapiens
AIVMTQSPDSLAVSLGERATINCKSSQSVLYSSNNKNYLAWYQQKPGQPPNLLFYWASTRESGVPDRFSVSGSGTDFTLTISSLQAEDVAVYYCQQYYDSPLTFGGGTKVEIK

SEQ ID NO: 13 - DNA, 375 bp, Homo sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagtactgggagctacggggggttactactactactacggtatggacgtctggggccaagggaccacggtcaccgtctcctca

SEQ ID NO: 14 - protein, 125 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARVLGATGGYYYYYGMDVWGQGTTVTVSS

SEQ ID NO: 15 - DNA, 318 bp, Homo sapiens
tcctatgagctgactcagccaccctcagtgtccgtgtccccaggacagacagccagcatcacctgctctggagataaattgggggataaatatgcttgctggtatcagcagaagccaggccagtcccctgtgctggtcatctatcaagatatcaagcggccctcagggatccctgagcgattctctggctccaagtctgggaacacagccactctgaccatcagcgggacccaggctatggatgaggctgactattactgtcaggcgtgggacagcagcactgtggtattcggcggagggaccaagctgaccgtccta

SEQ ID NO: 16 - protein, 106 aa, Homo sapiens
SYELTQPPSVSVSPGQTASITCSGDKLGDKYACWYQQKPGQSPVLVIYQDIKRPSGIPERFSGSKSGNTATLTISGTQAMDEADYYCQAWDSSTVVFGGGTKLTVL

SEQ ID NO: 17 - DNA, 351 bp, Homo sapiens
caggtgcagctggtggagtctgggggaggcgtggtccagcctgggaggtccctgagactctcctgtgcagcgtctggattcagcttcagtagctatggcatgcactgggtccgccaggctccaggcaaggggctggactgggtggcaattatatggtatgatggaagttataaatactatgcagactccgtgaagggccgattcaccatctccagagacaattccaagaacacgctgtctctgcaaatgaacagcctgagagccgaggacacggctgtatattactgtgcgagagatggaaagtatccctttgactactggggccagggaaccctggtcaccgtctcctca

SEQ ID NO: 18 - protein, 117 aa, Homo sapiens
QVQLVESGGGVVQPGRSLRLSCAASGFSFSSYGMHWVRQAPGKGLDWVAIIWYDGSYKYYADSVKGRFTISRDNSKNTLSLQMNSLRAEDTAVYYCARDGKYPFDYWGQGTLVTVSS

SEQ ID NO: 19 - DNA, 324 bp, Homo sapiens
tcctatgtgctgactcagccaccctcggtgtcagtggccccaggacagacggccaggattacctgtgggggaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatcgtagtagtgatcatgtggtattcggcggagggaccaagctgaccgtccta

SEQ ID NO: 20 - protein, 108 aa, Homo sapiens
SYVLTQPPSVSVAPGQTARITCGGNNIGSKSVHWYQQKPGQAPVLVVYDDSDRPSGIPERFSGSNSGNTATLTISRVEAGDEADYYCQVWDRSSDHVVFGGGTKLTVL

SEQ ID NO: 21 - DNA, 348 bp, Homo sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgtgagagactatagtagtggctggtactggggccagggaaccctggtcaccgtctcctca

SEQ ID NO: 22 - protein, 116 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCVRDYSSGWYWGQGTLVTVSS

SEQ ID NO: 23 - DNA, 324 bp, Homo sapiens
tcctatgagctgacacagccaccctcggtgtcagtgtccccaggacaaacggccaggatcacctgctctggagatgccttgccaaaaaaatatgcttattggtaccagcagaagtcaggccaggcccctgtgttggtcatctatgaggacagcaaacgaccctccgggatccctgagagattctctggctccagctcagggacaatggccaccttgactatcagtggggcccaggtggaagatgaagctgactactcctgttactcaatagacagcagtgttaatcatgtggtattcggcggagggaccaagctgaccgtccta

SEQ ID NO: 24 - protein, 108 aa, Homo sapiens
SYELTQPPSVSVSPGQTARITCSGDALPKKYAYWYQQKSGQAPVLVIYEDSKRPSGIPERFSGSSSGTMATLTISGAQVEDEADYSCYSIDSSVNHVVFGGGTKLTVL

SEQ ID NO: 25 - DNA, 362 bp, Homo sapiens
caggtgcagctggtggagtctgggggaggcgtggtccagcctgggaggtccctgagactctcctgtgcagcgtctggattcaccttcagtgactatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagcagctggcttctactactactacggtatggacgtctggggccaagggaccacggtcaccgtctcctc

SEQ ID NO: 26 - protein, 121 aa, Homo sapiens
QVQLVESGGGVVQPGRSLRLSCAASGFTFSDYGMHWVRQAPGKGLEWVAVIWYDGSNKYYADSVKGRFTISRDNSKNTLYLQMNSLRAEDTAVYYCARAAGFYYYYGMDVWGQGTTVTVSS

SEQ ID NO: 27 - DNA, 336 bp, Homo sapiens
gatattgtgatgactcagtctccactctccctgcccgtcacccctggagagccggcctccatctcctgcaggtctagtcagagcctcctgtatagtaatggatacaactatttggattggtacctgcagaagccagggcagtctccacagctcctgatctatttgggttctaatcgggcctccggggtccctgacaggttcagtggcagtgggtcaggcacagattttacactgaaaatcagcagagtggaggctgaggatgttgggctttattactgcatgcgagctctacaaactccattcactttcggccctgggaccaaagtggatatcaaa

SEQ ID NO: 28 - protein, 112 aa, Homo sapiens
DIVMTQSPLSLPVTPGEPASISCRSSQSLLYSNGYNYLDWYLQKPGQSPQLLIYLGSNRASGVPDRFSGSGSGTDFTLKISRVEAEDVGLYYCMRALQTPFTFGPGTKVDIK

SEQ ID NO: 29 - DNA, 358 bp, Homo sapiens
caggagcagctggtggagtctgggggaggcgtggtccagcctgggaggtccctgagactctcctgtacagcctctggattcaccttcagtaactatggcatacactgggtccgccaggctccaggcaaggggctggagtgggtgacagttatatcatatgatggaagtaaaaaatactatgcagactccgtgaagggccgattcaccatctccagagacaattccaagaacacactgtatctgcaaatgaacagcctgagagctgaggacacggctgtgtattactgtgcgagagcgttttctactatggttcggggagttgaccactggggccagggaaccctggtcaccgtctcct

SEQ ID NO: 30 - protein, 120 aa, Homo sapiens
QEQLVESGGGVVQPGRSLRLSCTASGFTFSNYGIHWVRQAPGKGLEWVTVISYDGSKKYYADSVKGRFTISRDNSKNTLYLQMNSLRAEDTAVYYCARAFSTMVRGVDHWGQGTLVTVSS

SEQ ID NO: 31 - DNA, 321 bp, Homo sapiens
gacatccagatgacccagtctccatcctccctgtctgcatctgttggagacagagtcaccatcacttgccaggcgagtcaggacatttacaaatctttaaattggtatcagcagagaccagggaaagcccctaacctcctgatctacgatgcttccaatttggaaacaggggtcccatcaaggttcagtggaagtggatctgggacagattttactttcaccatcagcagcctgcagcctgaggattttgcaagatatttctgtcagcaatatgataatctccctctcactttcggcggtgggaccagggtggagatcaaa

SEQ ID NO: 32 - protein, 107 aa, Homo sapiens
DIQMTQSPSSLSASVGDRVTITCQASQDIYKSLNWYQQRPGKAPNLLIYDASNLETGVPSRFSGSGSGTDFTFTISSLQPEDFARYFCQQYDNLPLTFGGGTRVEIK

SEQ ID NO: 33 - DNA, 375 bp, Homo sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagcactgggagttaccgggggttactactactactacggtatggacgtctggggccaagggaccacggtcaccgtctcctca

SEQ ID NO: 34 - protein, 125 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARALGVTGGYYYYYGMDVWGQGTTVTVSS

SEQ ID NO: 35 - DNA, 318 bp, Homo sapiens
tcctatgagctgactcagccaccctcagtgtccgtgtccccaggacagacagccagcatcacctgctctggagataaattgggggataaatatgtttgttggtatcagcagaagccaggccagtcccctgtgttggtcatctatcaagatagcaagcggccctcagggatccctgagcgattctctggctccaactctgggaacacagccactctgaccatcagcgggacccaggctatggatgaggctgactattgctgtcaggcgtgggacagcagcactgtggtattcggcggagggaccaagctgaccgtccta

SEQ ID NO: 36 - protein, 106 aa, Homo sapiens
SYELTQPPSVSVSPGQTASITCSGDKLGDKYVCWYQQKPGQSPVLVIYQDSKRPSGIPERFSGSNSGNTATLTISGTQAMDEADYCCQAWDSSTVVFGGGTKLTVL

SEQ ID NO: 37 - DNA, 357 bp, Homo sapiens
gaggtgcaactgttggagtctgggggaggcttggtacagcctggggggtccctgagactctcctgtgcagcctctggattcacctttagcagctatgccatgagctgggtccgccaggctccagggaaggggctggagtgggtctcaactattagtggtggtggtcatagcacatactacgcagactccgtgaagggccggttcaccatctccagagacaattccaagaacacgctgtatctgcaaatgaacagcctgagagccgaggacacggccgtctattactgtgcgcgtatagcaccggctggtccccactttgactactggggccagggaaccctggtcaccgtctcctca

SEQ ID NO: 38 - protein, 119 aa, Homo sapiens
EVQLLESGGGLVQPGGSLRLSCAASGFTFSSYAMSWVRQAPGKGLEWVSTISGGGHSTYYADSVKGRFTISRDNSKNTLYLQMNSLRAEDTAVYYCARIAPAGPHFDYWGQGTLVTVSS

SEQ ID NO: 39 - DNA, 333 bp, Homo sapiens
aattttatgctgactcagccccactctgtgtcggagtctccggggaagacggtaactttctcctgcacccgcagcagtggcagcattgccagcaactttgtgcagtggtaccagcaacgcccgggcagttcccccaccactgtgatctatgaacataaccaaagaccctctggggtccctgatcggttctctggctccatcgacagctcctccagttctgcctccctcaccatctctggactgaagactgaggacgaggctgactactactgtcagttttatgatcgcaacagtcattgggtgttcggcggagggaccaagctgaccgtccta

SEQ ID NO: 40 - protein, 111 aa, Homo sapiens
NFMLTQPHSVSESPGKTVTFSCTRSSGSIASNFVQWYQQRPGSSPTTVIYEHNQRPSGVPDRFSGSIDSSSSSASLTISGLKTEDEADYYCQFYDRNSHWVFGGGTKLTVL

SEQ ID NO: 41 - protein, 120 aa, Homo sapiens; identical to SEQ ID NO: 2

SEQ ID NO: 42 - protein, 107 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPITFGQGTRLEIK

SEQ ID NO: 43 - protein, 116 aa, Homo sapiens
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARGATYFDYWGQGTLVTVSS

SEQ ID NO: 44 - protein, 108 aa, Homo sapiens
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHVVFGGGTKLTVL

SEQ ID NO: 45 - protein, 120 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARIAAAYYYGMDVWGQGTTVTVSS

SEQ ID NO: 46 - protein, 113 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPLTFGGGTKVEIK

SEQ ID NO: 47 - protein, 121 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARGATYYYYYGMDVWGQGTTVTVSS

SEQ ID NO: 48 - protein, 107 aa, Homo sapiens
SYELTQPPSVSVSPGQTASITCSGDKLGDKYACWYQQKPGQSPVLVIYQDSKRPSGIPERFSGSNSGNTATLTISGTQAMDEADYYCQAWDSSTAVVFGGGTKLTVL

SEQ ID NO: 49 - protein, 115 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDGYFDYWGQGTLVTVSS

SEQ ID NO: 50 - protein, 108 aa, Homo sapiens; identical to SEQ ID NO: 44

SEQ ID NO: 51 - protein, 115 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARYSSGWYWGQGTLVTVSS

SEQ ID NO: 52 - protein, 108 aa, Homo sapiens
SYELTQPPSVSVSPGQTARITCSGDALPKKYAYWYQQKSGQAPVLVIYEDSKRPSGIPERFSGSSSGTMATLTISGAQVEDEADYYCYSTDSSGNHVVFGGGTKLTVL

SEQ ID NO: 53 - protein, 121 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARAAGYYYYYGMDVWGQGTTVTVSS

SEQ ID NO: 54 - protein, 112 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPFTFGPGTKVDIK

SEQ ID NO: 55 - protein, 117 aa, Homo sapiens
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTMVRGVDYWGQGTLVTVSS

SEQ ID NO: 56 - protein, 107 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKVEIK

SEQ ID NO: 57 - protein, 118 aa, Homo sapiens
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARGGYYYGMDVWGQGTTVTVSS

SEQ ID NO: 58 - protein, 107 aa, Homo sapiens [sequence omitted]

SEQ ID NO: 59 - protein, 118 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKIAAAGYFDYWGQGTLVTVSS

SEQ ID NO: 60 - protein, 110 aa, Homo sapiens
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNWVFGGGTKLTVL